(19) United States
(12) Patent Application Publication
(10) Pub. No.: US 2012/0142027 A1
(43) Pub. Date: Jun. 7, 2012

(54) Compositions and Methods for Modulating the Immune Response and Identifying Immunomodulators

(75) Inventor: You-Me Kim, Pohang (KR)

(73) Assignee: WHITEHEAD INSTITUTE FOR BIOMEDICAL RESEARCH, Cambridge, MA (US)

(21) Appl. No.: 12/528,213

(22) PCT Filed: Feb. 22, 2008
(86) PCT No.: PCT/US2008/054809

§ 371 Date: Jun. 1, 2010

Related U.S. Application Data
(60) Provisional application No. 60/902,983, filed on Feb. 23, 2007.

(51) Int. Cl. G01N 21/64; G01N 21/76; G01N 33/566

(52) U.S. Cl. 435/7.21

(57) ABSTRACT

The present invention provides compositions and methods for the identification of immunomodulators. The invention further provides an isolated complex comprising an UNC93B polypeptide and an UNC93B-dependent TLR polypeptide and methods of use thereof. The invention further provides methods of modulating immune system activity and methods of treating diseases characterized by aberrant immune system activity.

### GOVERNMENT FUNDING STATEMENT

The United States Government has provided grant support utilized in the development of the present invention. In particular, grant number 5 R37AI033456 awarded by the National Institutes of Health, has supported development of this invention. The Government has certain rights in the invention.

### BACKGROUND OF THE INVENTION

A properly functioning immune system plays important roles in the body's defense against infections and in tumor immunosurveillance. However, inappropriate or excessive immune system activation can lead to adverse consequences. Recognition of self antigens by immune system effector cells and molecules leads to a broad spectrum of autoimmune diseases. Release of pro-inflammatory cytokines as a consequence of immune system activation is responsible for a number of severe local and systemic reactions. In addition, a number of chronic diseases are attributable at least in part to persistent inflammation. Accordingly, understanding and manipulating the activity of molecules involved in mediating immune responses is of great scientific and medical importance.

Toll-like receptors (TLRs) are involved in the recognition and processing of a variety of signals delivered by viral and microbial products and are key regulators of both innate and adaptive immune responses. (Janeway and Medzhitov, 2002; Takeda et al., 2003). TLRs sense the presence of molecules broadly conserved across microbial taxa, and TLR activation initiates the innate immune response by inducing the expression of antimicrobial genes and inflammatory cytokines. Activation of TLRs also enhances adaptive immunity through activation of dendritic cells. TLR-mediated recognition of microbial components by dendritic cells induces the expression of costimulatory molecules such as CD80/CD86, secretion of inflammatory cytokines and is responsible for the rearrangement of trafficking pathways of Class II MHC products (Akira et al., 2001; Iwasaki and Medzhitov, 2004).

TLRs are transmembrane receptors that include a ligand-binding domain and a cytoplasmic portion that contains a conserved cytoplasmic motif, the Toll/interleukin (IL)-receptor (TIR) domain. The TLR TIR domains interact with a variety of adaptor proteins, triggering activation of downstream protein kinases and different transcription factors such as NF-kB, leading to proinflammatory cytokine production.

There are 10 and 12 TLR paralogues in humans and mice, respectively. Both species have TLRs 1-9. Mice lack TLR10, but have TLR11, 12, and 13, which humans lack. TLRs have the capacity to sense the presence of a variety of distinct microbial components (Kawai and Akira, 2006; Takeda et al., 2003). For example, TLR4 recognizes lipopolysaccharides (LPS), a component of the Gram-negative bacterial outer membrane, while dsRNA, ssRNA and unmethylated bacterial DNA (CpG) engage TLR3, TLR7 and TLR9, respectively (Alexopoulou et al., 2001; Bauer et al., 2001; Diebold et al., 2004; Heil et al., 2004; Hemmi et al., 2000; Poltorak et al., 1998). These components are also known as pathogen associated molecular patterns (PAMPs).

Studies demonstrate that TLRs 3, 7 and 9 are localized intracellularly (Ahmad-Nejad et al., 2002; Latz et al., 2004; Leifer et al., 2004; Matsumoto et al., 2003; Nishiya and DeFranco, 2004). TLR9 resides in the ER prior to stimulation and reaches lysosomes only upon activation (Latz et al., 2004; Leifer et al., 2004). TLR9 remains sensitive to Endo H digestion even after activation (Latz et al., 2004; Leifer et al., 2004). The transmembrane domains of TLR7 and 9 and the cytosolic linker region between the transmembrane and TIR domain of TLR3 determine the intracellular localization for these TLRs (Barton et al., 2006; Funami et al., 2004; Kajita et al., 2006; Nishiya et al., 2005).

UNC93B is required for signaling by TLRs 3, 7, and 9, and the H412R mutation eliminates signaling via these TLRs (Tabeta et al., 2006). Furthermore, mice with the H412R mutation are highly susceptible to various viral and bacterial infections infection such as mouse cytomegalovirus (MCMV), Listeria monocytogenes and Staphylococcus aureus infection (Tabeta et al., 2006;). UNC93B deficiency has also been linked to the etiology of Herpes simplex virus-1 encephalitis (HSE) in human patients (Casrouge et al., 2006). Cells from patients with a mutation in the Unc93b1 gene show impaired cytokine production upon stimulation of TLRs 3, 7, 8 and 9 and are highly susceptible to various viral infections. The findings in mice and humans underscore the importance of UNC93B in proper functioning of UNC93B-dependent TLRs but do not explain how it acts.

The function of TLRs in various human diseases has been investigated by comparison of the incidence of disease among people having different polymorphisms in genes that participate in TLR signaling. These studies have shown that TLR function or lack thereof affects a number of specific diseases and clinical conditions (See, e.g., Cook, et al., Nat. Immunol., 5(10):975-9, 2004; Tabeta, et al., Proc Natl Acad Sci; 101(10):3516-21, 2004). However, understanding of the mechanisms of TLR signaling remains incomplete, and only a limited number of agents capable of modulating TLR activity are known. Accordingly, there is a need in the art for additional information about TLR signaling mechanisms. There is also a need in the art for new agents capable of modulating TLR activity and new approaches for the identification of such agents.

### SUMMARY OF THE INVENTION

Assay compositions for identifying an agent (or agents) that modulates signaling by an intracellular Toll-like receptor (TLR) are provided. In some embodiments, the assay composition comprises: (a) a first polypeptide comprising an UNC93B polypeptide; and (b) a second polypeptide comprising an UNC93B-dependent TLR polypeptide. In some embodiments, the assay composition is adapted to specifically identify an agent that modulates the physical interaction between the UNC93B polypeptide and the UNC93B-dependent TLR polypeptide. In some embodiments the first and second polypeptides are isolated polypeptides. In some embodiments the first and second polypeptides are within a cell. In some embodiments, the first polypeptide or the second polypeptide comprises an affinity tag. In some embodiments, the first polypeptide or the second polypeptide is attached to a solid support. In some embodiments, the first polypeptide comprises a first component of a proximity-dependent reporter system and the second polypeptide comprises a second component of the proximity-dependent reporter system. In some embodiments, the first polypeptide comprises a Resonance Energy Transfer (RET) donor and the second polypeptide comprises a RET acceptor, such that RET occurs when the UNC93B polypeptide and the TLR polypeptide physically interact with each other. In some embodiments, the first polypeptide further comprises a first fragment of a reporter protein and the second polypeptide further comprises a second fragment of the reporter protein, wherein the protein has a detectable activity when the fragments assemble. In some embodiments, the first polypeptide comprises a first component of a proximity-dependent reporter system, and wherein the assay composition comprises at least two polypeptides each comprising (i) an different UNC93B-dependent TLR polypeptide and (ii) a component of a proximity-dependent reporter system capable of interacting (e.g., physically interacting) with the first component of a proximity-dependent reporter system, wherein the UNC93B-dependent TLRs are different. In some embodiments, the first polypeptide comprises a first component of a proximity-dependent reporter system, and wherein the assay system comprises at least three polypeptides each comprising (i) an different UNC93B-dependent TLR polypeptide and (ii) a component of a proximity-dependent reporter system capable of interacting (e.g., physically interacting) with the first component of a proximity-dependent reporter system, wherein the UNC93B-dependent TLRs are different. In some embodiments, the UNC93B and TLR sequences are human. In some embodiments, any of the compositions further comprises a candidate agent.

Also provided are assay compositions having any combination of the foregoing features, provided such features are not mutually inconsistent. For example, a polypeptide could comprise an affinity tag and be attached to a solid support.

Assay systems are also provided comprising any of the afore-mentioned assay compositions and means for detecting a signal produced by said assay composition in the presence of an agent that modulates the physical interaction between UNC93B polypeptide and the UNC93B-dependent TLR polypeptide.

Cells expressing a recombinant UNC93B polypeptide and a recombinant UNC93B-dependent TLR polypeptide are also provided. Further provided are cells that express a recombinant polypeptide comprising UNC93B polypeptide and/or express recombinant polypeptide(s) comprising one or more UNC93B-dependent TLR polypeptides described herein (e.g., further comprising a reporter protein fragment, RET donor or acceptor, tag, etc.) Further provided are nucleic acid constructs encoding the recombinant polypeptides and expression vectors comprising said recombinant polypeptides. Methods of making the nucleic acid constructs, vectors, and cells are also provided.

For example, nucleic acid constructs that encode an UNC93B polypeptide or an UNC93B-dependent TLR polypeptide, wherein said polypeptide is fused to a first fragment of a reporter protein, wherein said first fragment lacks at least one detectable activity of the reporter protein but wherein said first fragment and a second fragment can assemble to reconstitute a reporter protein having said activity under suitable conditions are provided.

Assays that are adapted to specifically detect a physical interaction between an UNC93B polypeptide and an UNC93B-dependent TLR polypeptide are provided. In one embodiment, the assay comprises the steps of: (a) isolating an UNC93B polypeptide from cells, and (b) determining whether said isolating step also results in isolation of an UNC93B-dependent TLR polypeptide. In some embodiments, the assay comprises the steps of: (a) isolating an UNC93B-dependent TLR polypeptide from cells, and (b) determining whether said isolating step also results in isolation of an UNC93B polypeptide. In some embodiments, said isolating comprises contacting a cell lysate with an agent that specifically binds to UNC93B polypeptide or the UNC93B-dependent TLR polypeptide.

Methods of identifying an immunomodulator are also provided. In some embodiments, the method comprises the steps of: (a) performing an interaction-specific assay that assesses the ability of a candidate agent to modulate interaction between an UNC93B polypeptide and an UNC93B-dependent TLR polypeptide, and (b) identifying the candidate agent as an immunomodulator if it modulates the interaction between the UNC93B polypeptide and the UNC93B-dependent TLR polypeptide. In some embodiments, the interaction-specific assay is a protein complementation assay. In some embodiments, the interaction-specific assay is a RET-based assay. In some embodiments, the interaction-specific assay is a binding assay. In some embodiments the method comprises (a) identifying a candidate agent that modulates TLR signaling by performing an interaction-independent assay that is capable of assessing whether a candidate agent modulates TLR signaling, and then (b) performing an interaction-specific assay that assesses the ability of the candidate agent identified in step (a) to modulate interaction between UNC93B and an UNC93B-dependent TLR, and (c) identifying the candidate agent as an immunomodulator if it modulates the interaction between UNC93B and the UNC93B-dependent TLR. In some embodiments, the method comprises the steps of: (a) providing a composition comprising an UNC93B polypeptide, an UNC93B-dependent TLR polypeptide, and a candidate agent, (b) contacting the composition with a candidate agent under conditions suitable for a physical interaction to occur between the UNC93B polypeptide and the TLR polypeptide, and (c) determining whether the agent alters the physical interaction that would, in the absence of the candidate agent, be expected to occur between the UNC93B polypeptide and the TLR polypeptide. In some embodiments, the method comprises contacting cells that express the UNC93B polypeptide and the UNC93B-dependent TLR polypeptide with the candidate agent. In some embodiments, the UNC93B polypeptide and the UNC93B-dependent TLR polypeptide are at least partially purified. The TLR may be selected from the group consisting of: TLR3, TLR7, TLR8, and TLR9. Assays may employ any subset of these TLRs.

The invention also provides a method of identifying an immunomodulator that modulates signaling by an UNC93B dependent TLR, comprising the steps of: (a) contacting a cell that expresses both an UNC93B polypeptide and an UNC93B-dependent TLR polypeptide with a candidate agent; and (b) determining whether the candidate agent affects the localization of the UNC93B polypeptide or the TLR polypeptide, wherein an agent that affects the localization of the UNC93B polypeptide or the TLR polypeptide is identified as a potential immunomodulator that modulates signaling by an UNC93B-dependent TLR.

Methods of identifying an immunomodulator that modulates signaling by at least two UNC93B-dependent TLRs are also provided. In some embodiments, the method comprises the steps of: (a) providing an agent that alters the physical interaction between an UNC93B polypeptide and a first UNC93B-dependent TLR polypeptide, and (b) determining whether the agent modulates the physical interaction between an UNC93B polypeptide and a second UNC93B-dependent TLR polypeptide. In some embodiments, the method further comprises determining whether the agent alters the physical interaction between an UNC93B polypeptide and a third UNC93B-dependent TLR polypeptide. Further provided is a method of modulating an activity of an immune system cell comprising contacting the cell with an agent identified according to any of the afore-mentioned methods. Further provided is a method of modulating an activity of an immune system of a subject comprising administering to the subject an agent identified according to any of the afore-mentioned methods. In some embodiments, the subject suffers from or is at increased risk of an autoimmune disease, infection, or transplant rejection.

Further provided is a method of characterizing an agent comprising: (a) contacting an assay composition comprising an UNC93B polypeptide and an UNC93B-dependent TLR polypeptide with the agent, (b) detecting a complex comprising the UNC93B polypeptide and the UNC93B-dependent TLR polypeptide, and (c) comparing the amount of the complex with a reference value, wherein a difference between the amount of the complex and the reference value is indicative that the agent modulates complex formation. The agent may be a known immunomodulator.

Further provided is an isolated complex comprising an UNC93B polypeptide and an UNC93B-dependent TLR polypeptide. In some embodiments, the UNC93B polypeptide, the UNC93B-dependent TLR polypeptide, or both comprises a polypeptide portion that is not found joined to the polypeptide in nature. In some embodiments, the polypeptide portion is a fragment of a reporter protein, a RET donor or acceptor, an epitope tag, etc.

Further provided is a method of monitoring the effect of an agent on immune system cells comprising: (a) providing immune system cells, and (b) detecting a complex comprising UNC93B and an UNC93B-dependent TLR in the cells or in a sample obtained from the cells; and, optionally (c) comparing the amount of the complex with a reference value.

Methods of evaluating the immune system of a subject are also provided. In some embodiments, the methods comprise: (a) providing a sample obtained from the subject, wherein the sample comprises immune system cells, (b) detecting a complex comprising UNC93B and an UNC93B-dependent TLR in the sample, and optionally (c) comparing the amount of the complex with a reference value. In some embodiments, detecting an altered amount of the complex relative to a reference value indicates that altered complex formation between UNC93B and an UNC93B-dependent TLR is a feature of the disorder.

Further provided is a method of modulating an activity of an immune system cell comprising contacting the cell with an agent that modulates interaction between UNC93B and an UNC93B-dependent TLR. Further provided is a method of modulating an activity of the immune system of a subject comprising administering to the subject an agent that modulates interaction between UNC93B and an UNC93B-dependent TLR. In some embodiments, the subject suffers from or is at increased risk of an autoimmune disease, infection, or transplant rejection.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Such techniques are explained fully in the literature.

Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005. In the event of a conflict or inconsistency between any of the literature and the instant specification, the specification shall control. Standard art-accepted meanings of terms are used herein unless indicated otherwise. Standard abbreviations for various terms are used herein.

### BRIEF DESCRIPTION OF THE DRAWINGS

### Examples 1-8

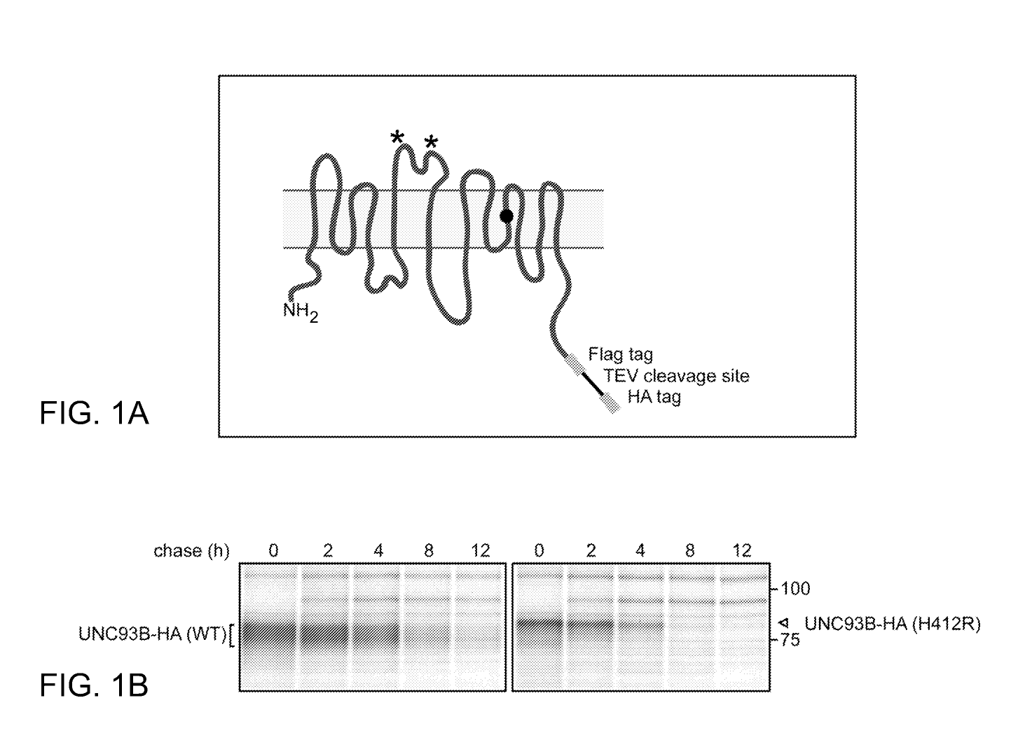

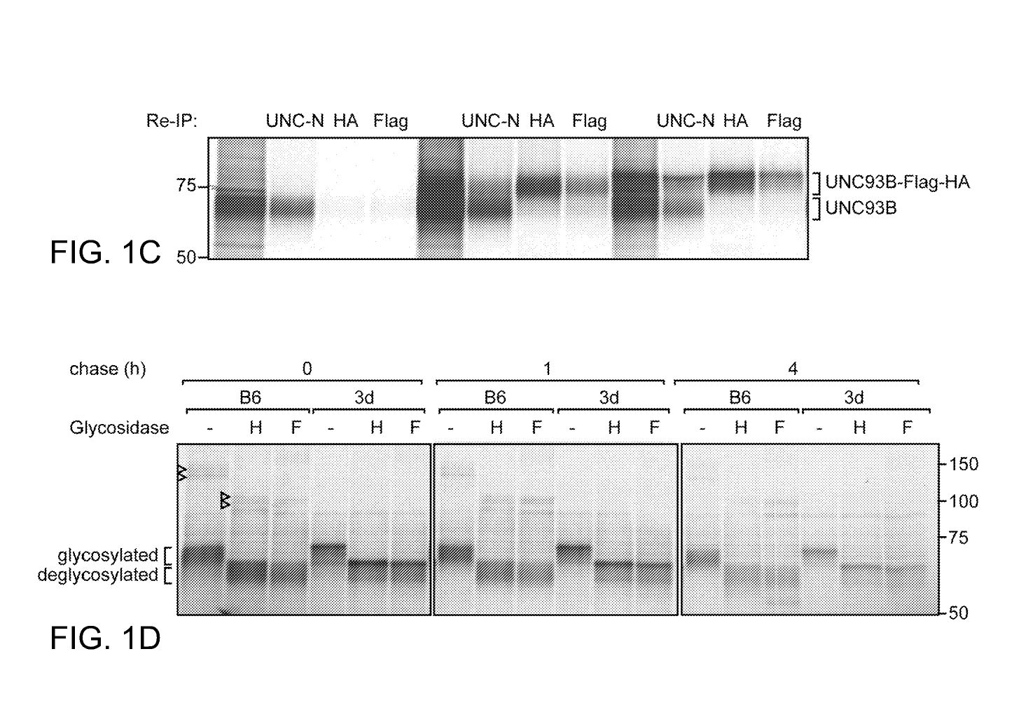

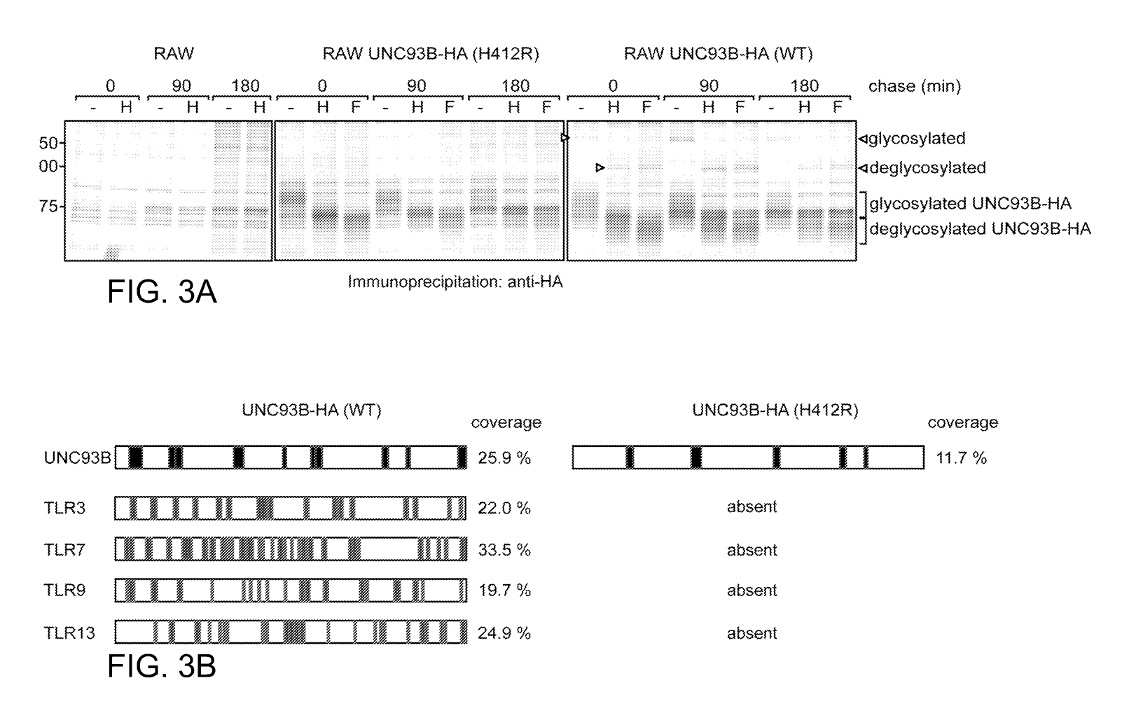

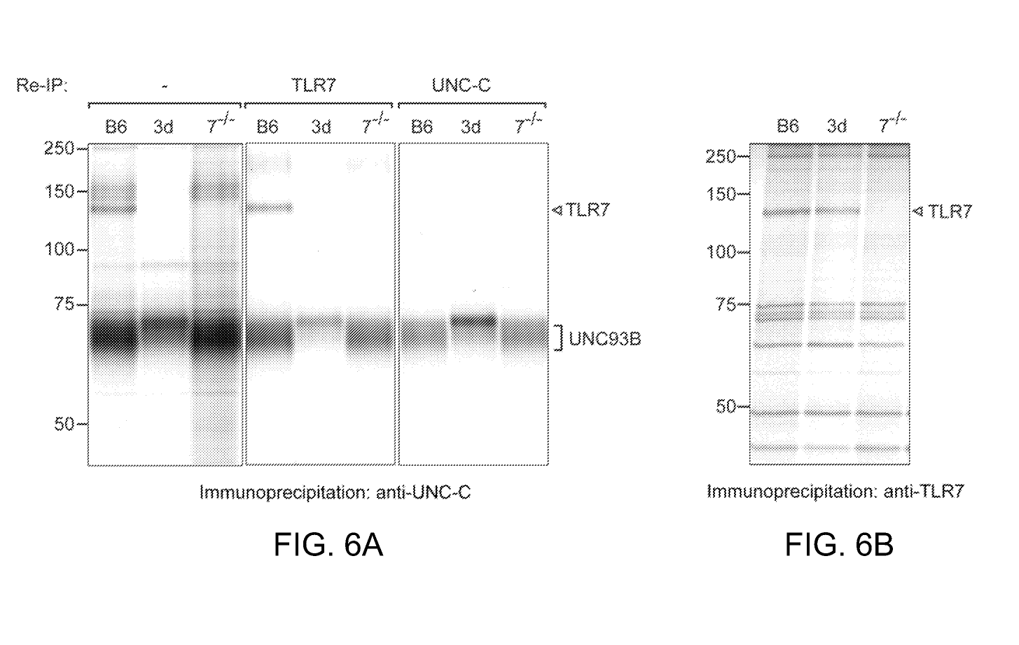

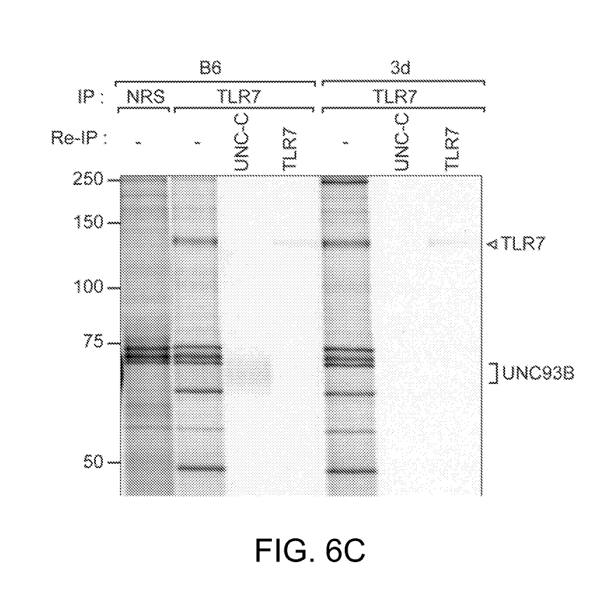

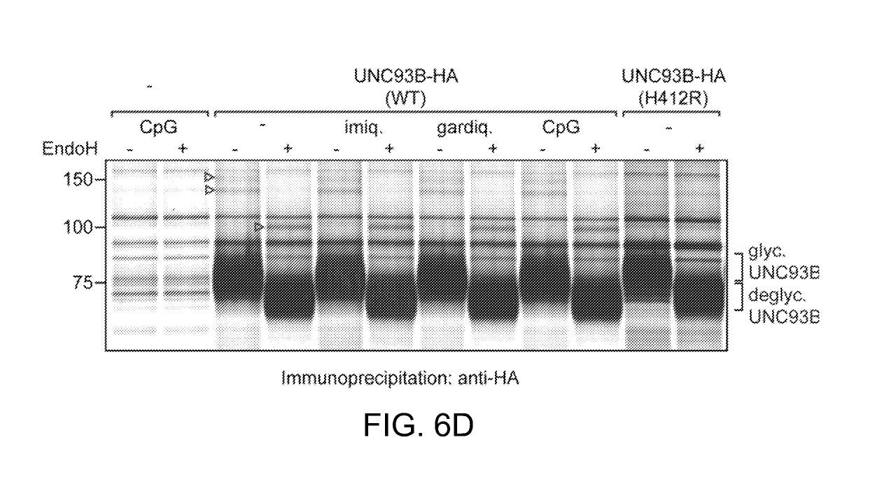

FIG. 1. Characterization of wild type and mutant UNC93B proteins. (a) Model of the UNC93B protein. The single point mutation of histidine residue 412 to arginine (H412R, 3d mutation) is located within transmembrane domain 9 (•). The two predicted N-linked glycosylation sites (N²⁵¹HT and N²⁷²KT) are indicated (*). Wild type and mutant (H412R) UNC93B were fused at the C-terminus with a Flag-tag, followed by the TEV protease cleavage site and an HA-tag (designated as UNC93B-HA). (b) RAW macrophages stably expressing epitope-tagged wild type UNC93B-HA (WT, left panel) or mutant UNC93B-HA (H412R, right panel) were metabolically labeled with ³⁵S-methionine/cysteine for 30 min (pulse) and lysed in RIPA buffer after 0, 2, 4, 8 and 12 h of incubation in normal medium (chase). UNC93B proteins were recovered by immunoprecipitation with an anti-HA antibody and resolved by SDS-PAGE. Wild type and mutant UNC93B proteins show similar stability, but differ in their migration patterns: Wild type UNC93B migrates as hetero-disperse material upon SDS-PAGE, whereas the UNC93B mutant form migrates as a well-defined distinct polypeptide in addition to more diffuse material. (c) Endogenous and epitope-tagged UNC93B proteins were immunoprecipitated in 1% NP40 lysis buffer from ³⁵S-labeled RAW macrophages that are non-transduced (−) or transduced with UNC93B-HA (WT) or UNC93B-HA (H412R) using a polyclonal antibody directed against the C-terminus of UNC93B (anti-UNC-C). Re-immunoprecipitations were carried out after mild denaturation of the initial immunoprecipitation with the following antibodies: a polyclonal antiserum against the N-terminus of UNC93B (anti-UNC-N), or antibodies to the HA or Flag epitopes. Recovered epitope-tagged and endogenous UNC93B proteins were resolved by SDS-PAGE. The distinct polypeptide observed in direct immunoprecipitations of mutant UNC93B is also observed after re-immunoprecipitation of mutant UNC93B and is thus UNC93B itself. (d) Bone marrow-derived dendritic cells from either wild type C57BL/6 (B6) or UNC93B mutant (3d) mice were pulsed with ³⁵S-methionine/cysteine for 40 min and lysed in 1% digitonin lysis buffer after 0, 1 or 4 h of incubation in normal medium (chase). Endogenous UNC93B proteins were immunoprecipitated with the affinity purified anti-UNC-C antibody, subjected to digestion with glycosidases Endo H(H) or PNGase F (F) or not digested (−), and resolved by SDS-PAGE. Both wild type and mutant UNC93B proteins retain full sensitivity to Endo H digestion. The polypeptides migrating at the range of 130˜450 kDa in non-digested samples and at ˜400 kDa in Endo H/PNGase F digested samples (indicated by arrowheads) are only present in wild type samples, but not in the 3d samples (see below and FIGS. 3a and 6a).

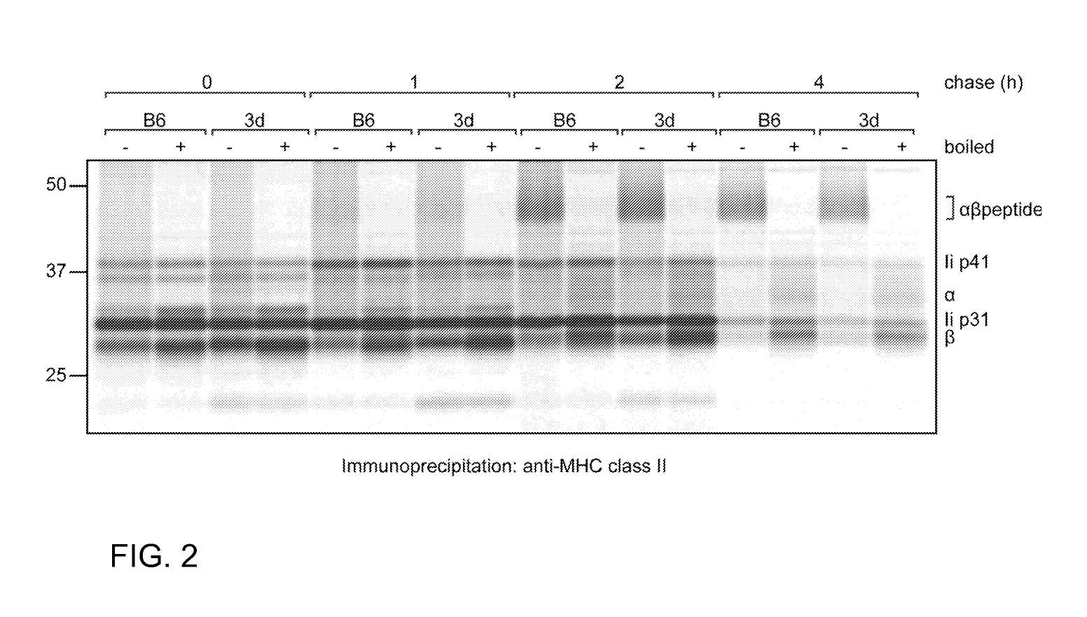

FIG. 2. Maturation of MHC class II is not altered in UNC93B mutant mice. Bone marrow-derived dendritic cells from either wild type C57BL/6 (B6) or UNC93B mutant (3d) mice were pulsed with ³⁵S-methionine/cysteine for 40 min and lysed in 1% digitonin lysis buffer after 0, 1, 2 or 4 h of incubation in normal medium (chase). Endogenous MHC class II was immunoprecipitated with the anti-MHC class II antibody (N22). Immunoprecipitates were loaded under denaturing (boiled) or mildly denaturing (non-boiled) conditions and resolved by SDS-PAGE. SDS-stable class II MHC complexes are composed of the α/β heterodimer and antigenic peptide (αβ peptide). Ii; invariant chain.

FIG. 3. TLRs 3, 7, 9 and 13 bind to wild type but not mutant UNC93B. (a) RAW macrophages (left panel) or RAW cells stably transduced with either mutant (middle panel) or wild type (right panel) UNC93B-HA were labeled with ³⁵S-methionine/cysteine for 30 min. Cells were lysed in 1% digitonin lysis buffer after chase periods of 0, 90 or 180 min and UNC93B-HA was immunoprecipitated with an anti-HA antibody. The samples were incubated with Endo H(H) or PNGase F (F) and resolved by SDS-PAGE. The polypeptide (˜130 kDa in its glycosylated form and ˜100 kDa in its deglycosylated form) that coimmunoprecipitated with wild type UNC93B is indicated with arrowheads. (b) Four billion RAW cells expressing wild type (WT) or mutant (H412R) UNC93B-HA were lysed in 1% digitonin buffer and UNC93B-HA proteins were immunoprecipitated with an anti-HA antibody. After immunoprecipitation, UNC93B and UNC93B-associated proteins were released by incubation with TEV protease, resolved by SDS-PAGE and visualized by silver staining. Polypeptides were excised from the gel and analyzed by LC/MS/MS after trypsin digestion. Peptides for UNC93B (shaded in black) were recovered in both samples, while peptides corresponding to sequences of TLRs 3, 7, 9 and 13 (shaded in grey) were identified only in the sample containing wild type UNC93B. Peptide sequences identified by mass spectrometry for UNC93B and TLRs are given in Tables 2 and 3, respectively. A full list of proteins that are coimmunoprecipitated with wild type or mutant UNC93B and identified by LC/MS/MS is given in Table 4.

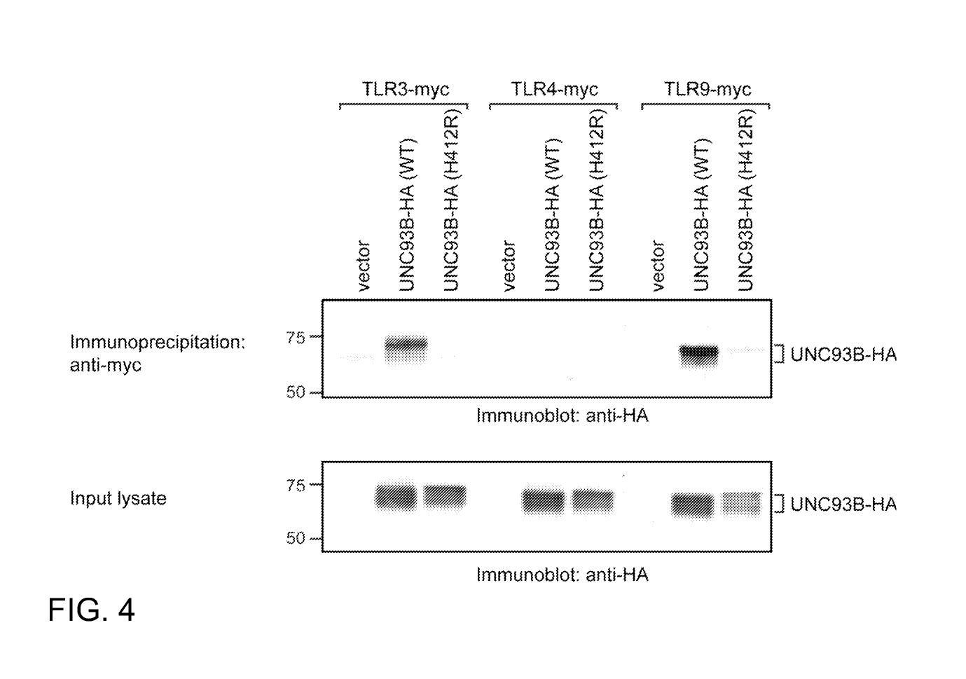

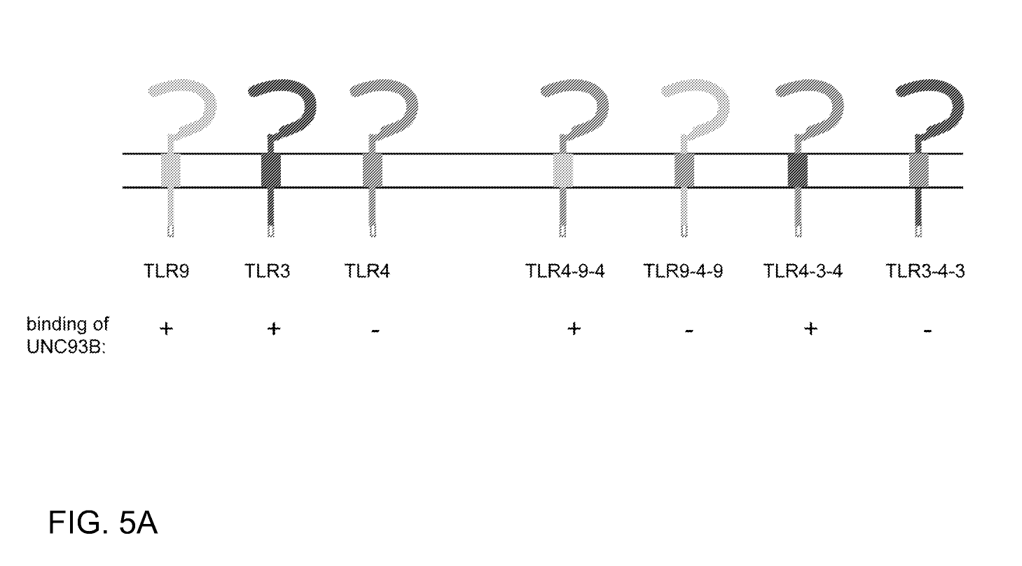

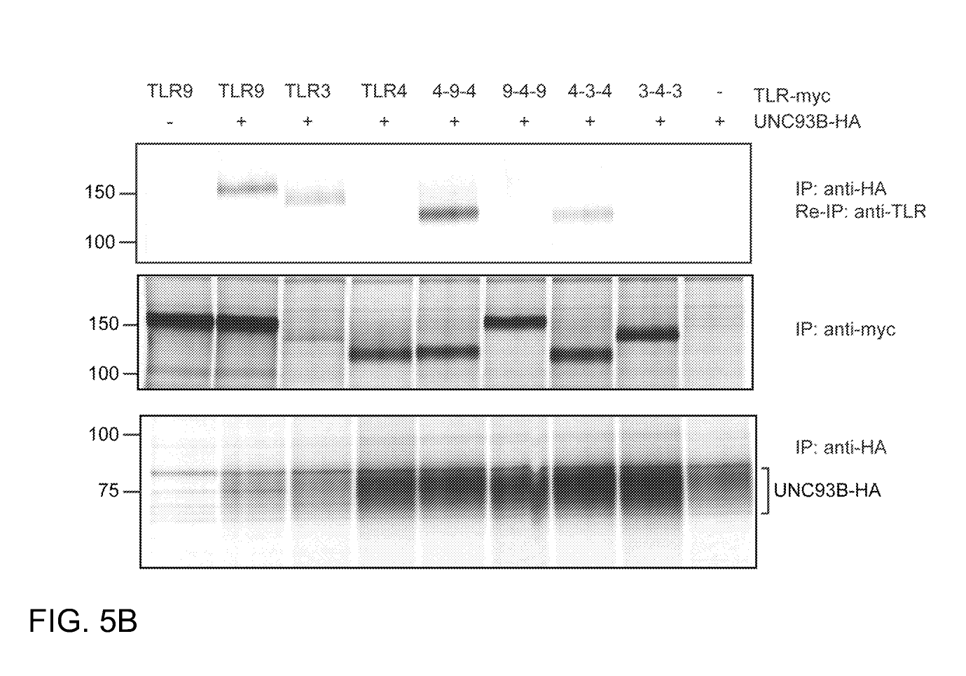

FIG. 4. Wild type UNC93B associates with TLR3 and TLR9, but not with TLR4. HEK 293-T cells were co-transfected with empty vector or expression constructs for wild type or mutant UNC93B-HA along with expression constructs for myc-tagged TLR3, TLR4 or TLR9 (see FIG. 5a). Cells were lysed with 1% digitonin lysis buffer and TLRs were immunoprecipitated with an anti-myc antibody. Samples were resolved by SDS-PAGE and UNC93B proteins were detected by immunoblotting using an anti-HA antibody (upper panel). Wild type UNC93B was recovered by immunoprecipitation of TLRs 3 and 9, whereas the mutant UNC93B (H412R) did not co-immunoprecipitate with any of TLRs. Input lysates were analyzed by SDS-PAGE for expression levels of wild type and mutant UNC93B-HA with an anti-HA antibody (lower panel).

FIG. 5. TLR3 and TLR9 interact with UNC93B via their transmembrane segments. (a) Schematic presentation of myc-tagged TLR expression constructs used in this study. The myc-tag was fused to the C-terminus of the TLRs and is indicated as a white rectangle. The TLR chimeras were generated such that the transmembrane segments were exchanged between TLRs 3, 4 and 9 to yield TLR3 and TLR9 with the transmembrane segment of TLR4 (TLR3-4-3 and TLR9-4-9), and TLR4 with the transmembrane segment of either TLR3 (TLR4-3-4) or TLR9 (TLR4-9-4). Binding capabilities of wild type UNC93B to TLRs as shown in this study (b and c) are indicated (−: no binding, +: binding). HEK 293-T cells were co-transfected with an empty vector (−) or an expression construct for wild type UNC93B-HA together with myc-tagged TLR3, 4 or 9 (b), or the myc-tagged TLR chimeras TLR9-4-9, 4-9-4, 4-3-4, 3-4-3 (c). Cells were metabolically labeled for 2 h with ³⁵S-methionine/cysteine and lysed in 1% digitonin lysis buffer. One third of the lysate was subjected to immunoprecipitation with an anti-myc antibody, and the rest was subjected to immunoprecipitation with an anti-HA antibody. Both immunoprecipitations were resolved by SDS-PAGE. Polypeptides corresponding to TLRs 3, 4 and 9 (b) and the different TLR chimeras (c) are indicated by arrowheads. TLR3 and TLR9 were recovered in UNC93B-HA immunoprecipitates, as well as the TLR chimeras TLR4-3-4 and TLR4-9-4, but not TLR4 and chimeras TLR9-4-9 and TLR3-4-3.

FIG. 6. Endogenous UNC93B and TLR7 associate in bone marrow-derived dendritic cells from wild type, but not from UNC93B mutant mice. (a˜c) Day 5 cultures of BM-DCs prepared from wild type (C57BL/6, B6), UNC93B mutant (3d) or TLR7 deficient (TLR7^{-/-}) mice were metabolically labeled for 4 h with ³⁵S-methionine/cysteine and lysed in 1% digitonin lysis buffer. (a) The first immunoprecipitation was performed with the purified anti-UNC-C antibody (left panel). After mild denaturation of the initial immunoprecipitates, re-immunoprecipitations were carried out with either an anti-TLR7 antibody (middle panel) or the anti-UNC-C antiserum (right panel). TLR7 was recovered with wild type, but not with mutant UNC93B. Expression levels of wild type and mutant UNC93B were comparable. (b) TLR7 expression levels in wild type (B6), UNC93B mutant (3d) and TLR7 knockout (TLR7^{-/-}) mice were analyzed by direct immunoprecipitation of TLR7 with the TLR7 antibody. Endogenous TLR7 is present at equal levels in both wild type and UNC93B mutant 3d mice, but absent in TLR7 knockout mice. (c) TLR7 was immunoprecipitated with the TLR7 antibody from digitonin lysates of BM-DCs from wild type (B6) or UNC93B mutant (3d) mice. Immunoprecipitation with normal rabbit serum (NRS) served as a control for TLR7 immunoprecipitation. Re-immunoprecipitations with anti-UNC-C or anti-TLR7 antibodies were performed after mild denaturation of the initial TLR7 immunoprecipitates and resolved by SDS-PAGE. UNC93B was coimmunoprecipitated with TLR7 in BM-DC lysates from wild type, but not from UNC93B mutant mice. Recovery of TLR7 from BM-DCs of both wild type and mutant mice by immunoprecipitation and re-immunoprecipitation was equal. (d) Non-transduced A20 B cells (−) or A20 B cells stably transduced with wild type (WT) or mutant UNC93B-HA (H412R) were metabolically labeled for 4 h with ³⁵S-methionine/cysteine (pulse) and stimulated with TLR7 agonists imiquimod (10 μM) or gardiquimod (1 μM) or the TLR9 agonist CpG DNA (1 μM) for 1 h during the final hour of the pulse. Cells were lysed in 1% digitonin lysis buffer and immunoprecipitations were carried out with an anti-HA antibody. Samples were not treated (−) or digested with Endo H (+) and resolved by SDS-PAGE. The polypeptides that associate only with wild type UNC93B and have characteristics of TLRs are indicated by arrowheads. Interaction of these polypeptides with wild type UNC93B was not significantly changed in stimulated cells compared to non-stimulated cells.

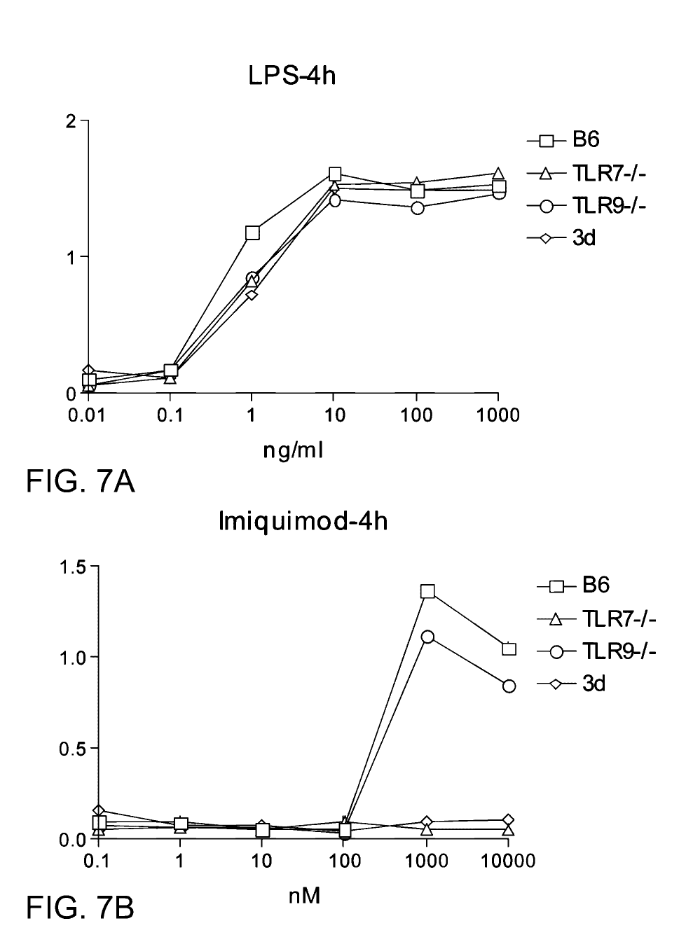

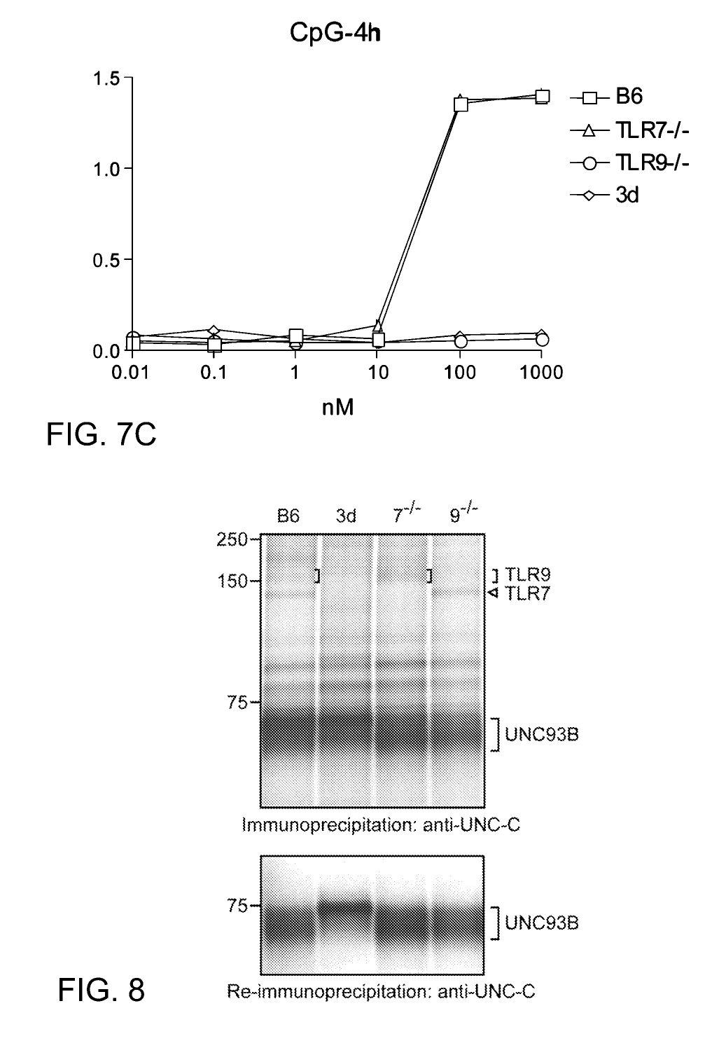

FIG. 7. TNF secretion in response to TLR agonists in BM-DCs from wild type versus UNC93B mutant mice. BM-DCs derived from either wild type (C57BL/6, B6), UNC93B mutant (3d), TLR7 knockout (TLR7^{-/-}) or TLR9 knockout (TLR9) mice were stimulated for 4 h with increasing concentrations of TLR agonists LPS (TLR4), imiquimod (TLR7) or CpG DNA (TLR9). After stimulation, the culture supernatant was collected and analyzed for tumor necrosis factor (TNF) by ELISA.

FIG. 8. TLR7 and TLR9 coimmunoprecipitate with wild type UNC93B in splenocytes from wild type, but not UNC93B mutant mice. Splenocytes prepared from wild type (C57BL/6, B6), UNC93B mutant (3d), TLR7 knockout (TLR7^{-/-}) or TLR9 knockout (TLR9^{-/-}) mice were metabolically labeled for 4 h with ³⁵S-methionine/cysteine and lysed in 1% digitonin lysis buffer. The first immunoprecipitation was performed with the purified anti-UNC-C antibody (upper panel). The polypeptides corresponding to TLR7 and TLR9 are indicated with an arrowhead and a bracket, respectively. After mild denaturation of the initial immunoprecipitates, UNC93B proteins were re-immunoprecipitated with the anti-UNC-C antiserum (lower panel).

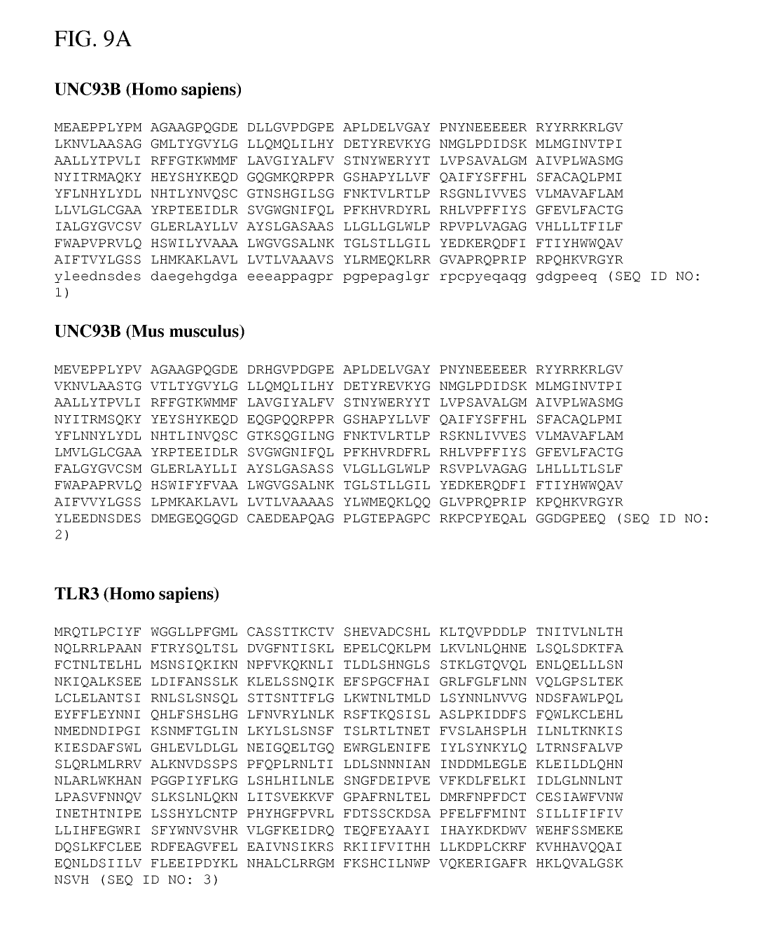

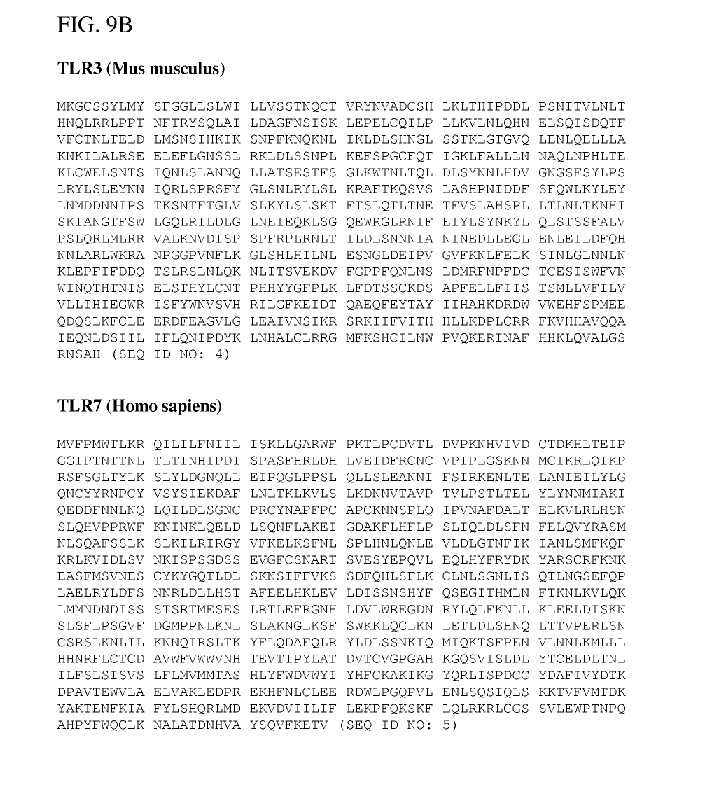

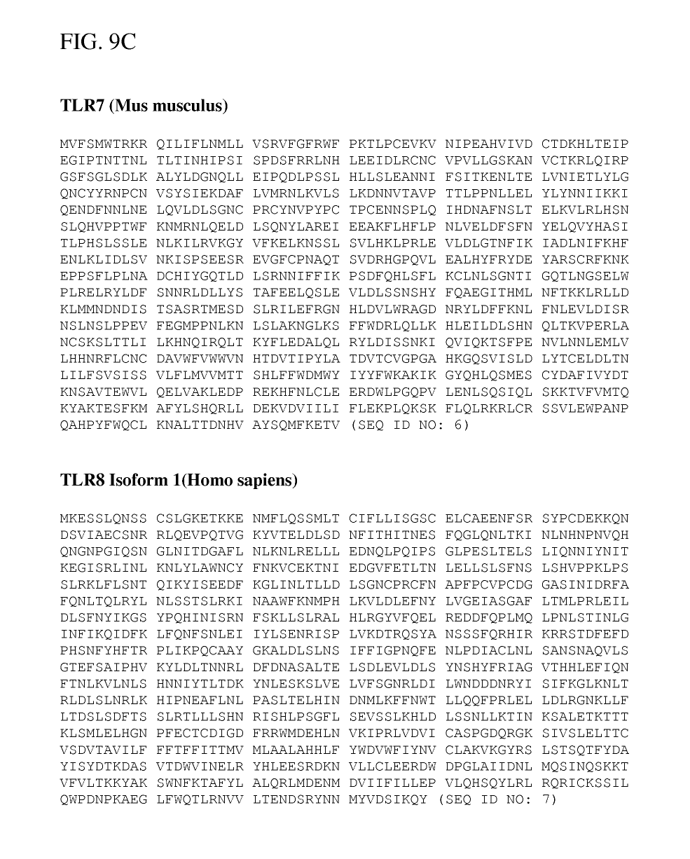

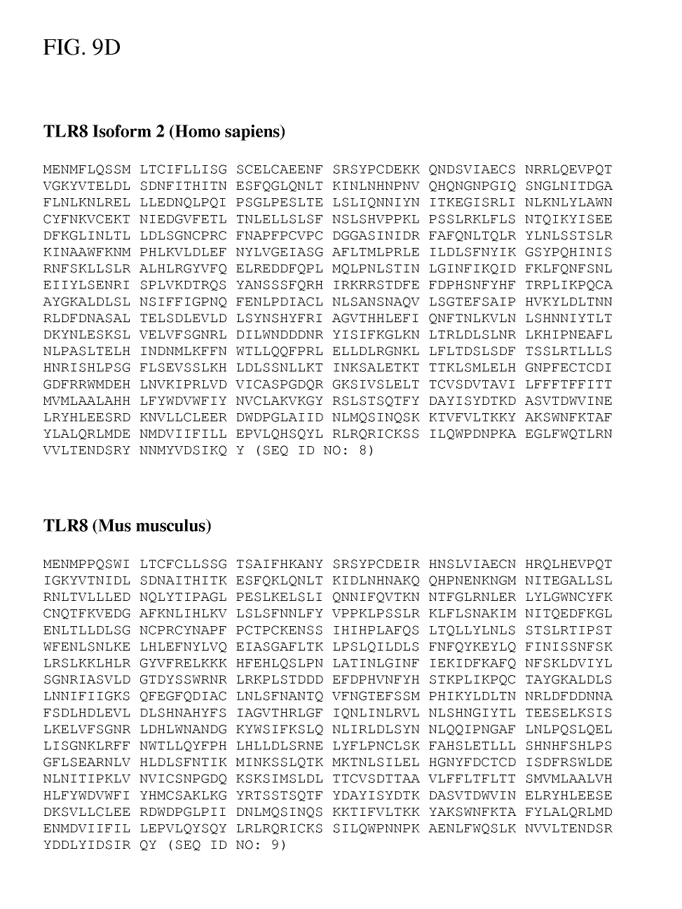

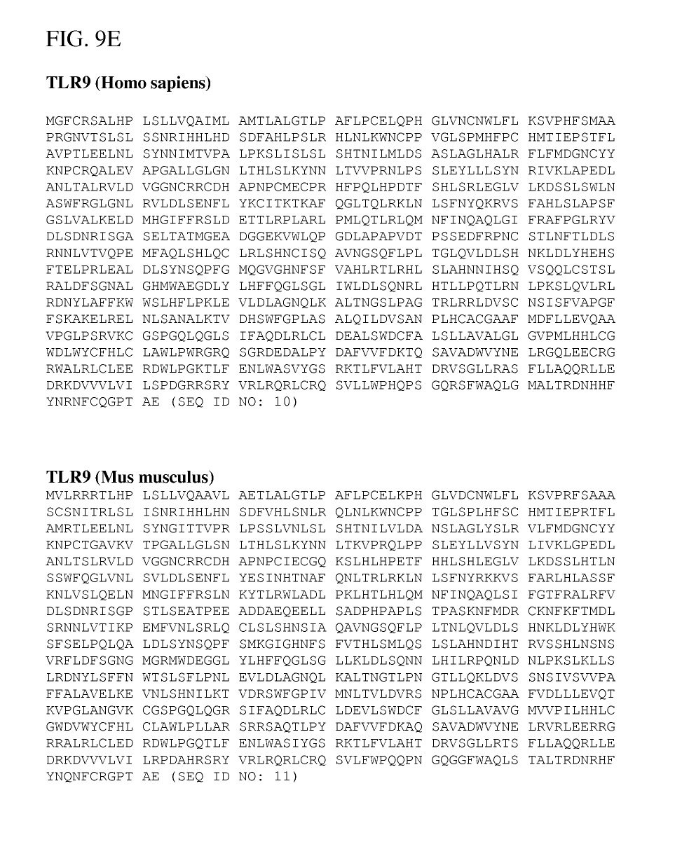

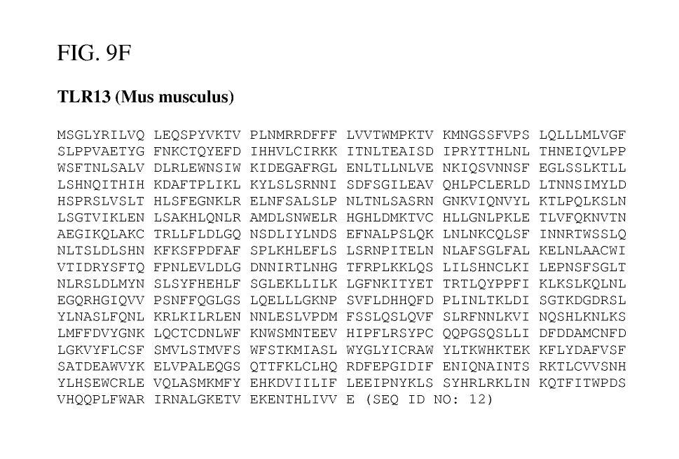

FIG. 9. Exemplary sequences of human and murine UNC93B and UNC93B-dependent TLRs.

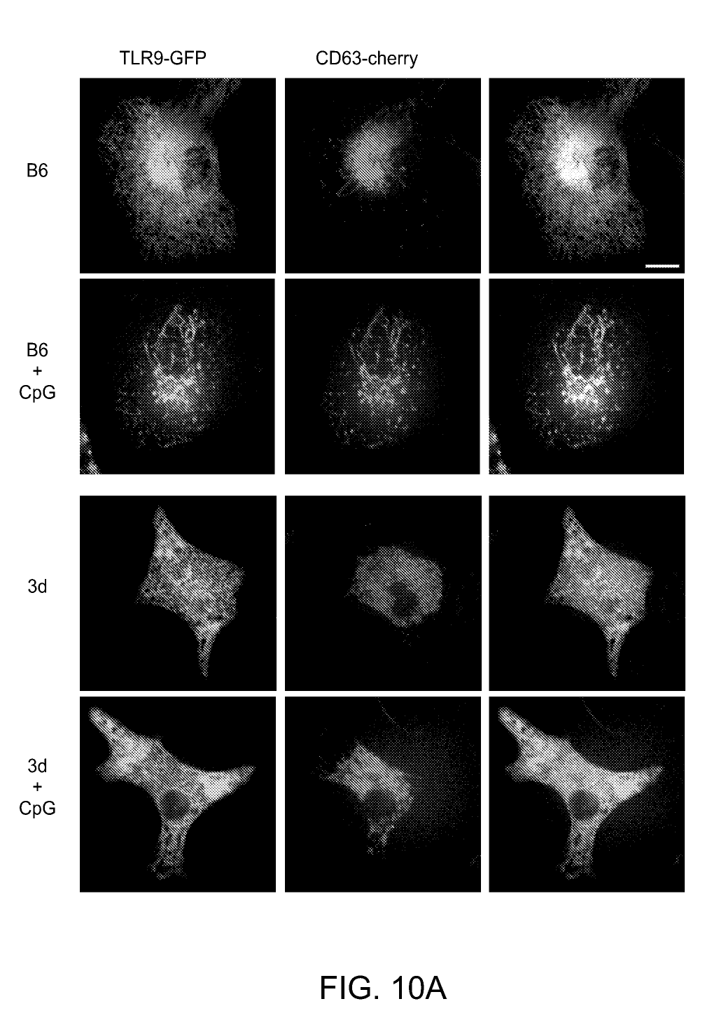

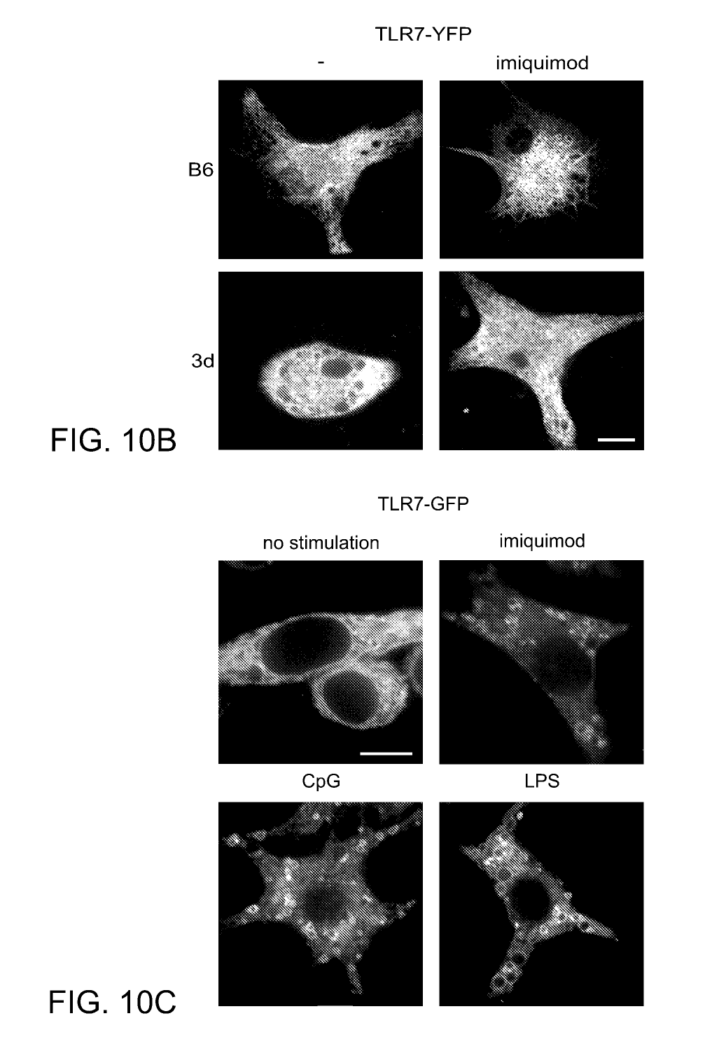

FIG. 10. TLR7 and TLR9 fail to translocate to endolysosomes in 3d cells. a and b, TLR9-GFP and CD63-cherry (a) or TLR7-YFP (b) were expressed in either B6 or 3d BM-DCs and live cells were imaged before and after stimulation with 1 μM CpG (a) or 1 μM imiquimod (b) for 2 h. c, RAW macrophages stably expressing TLR7-GFP were stimulated with 1 μM imiquimod, 1 μM CpG or 1 μg/ml LPS or left unstimulated. Live cells were imaged after 4 h of agonist stimulation. Scale bar=10 μm

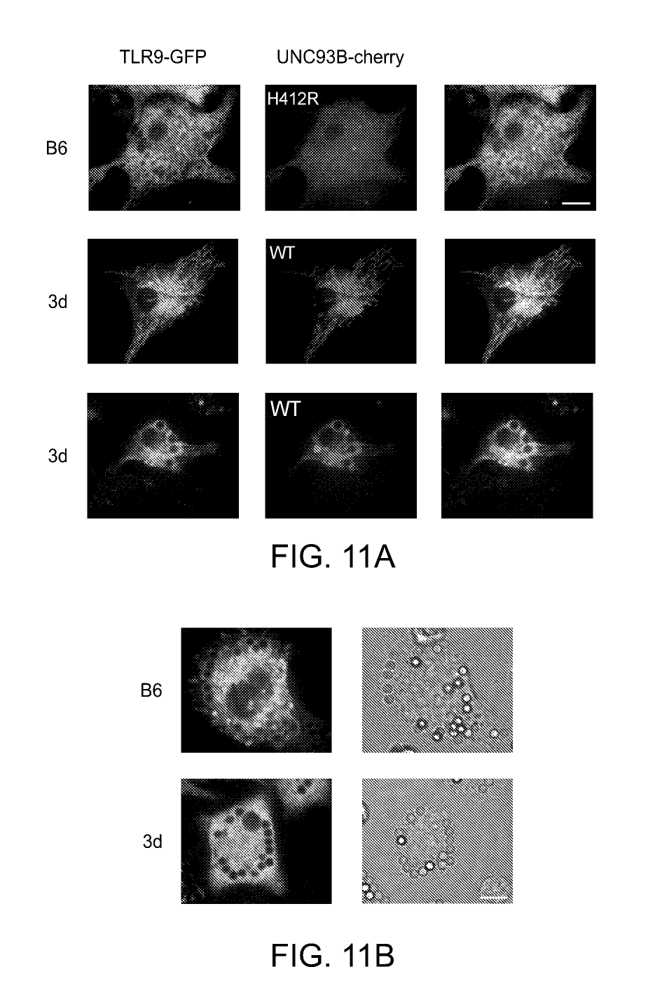

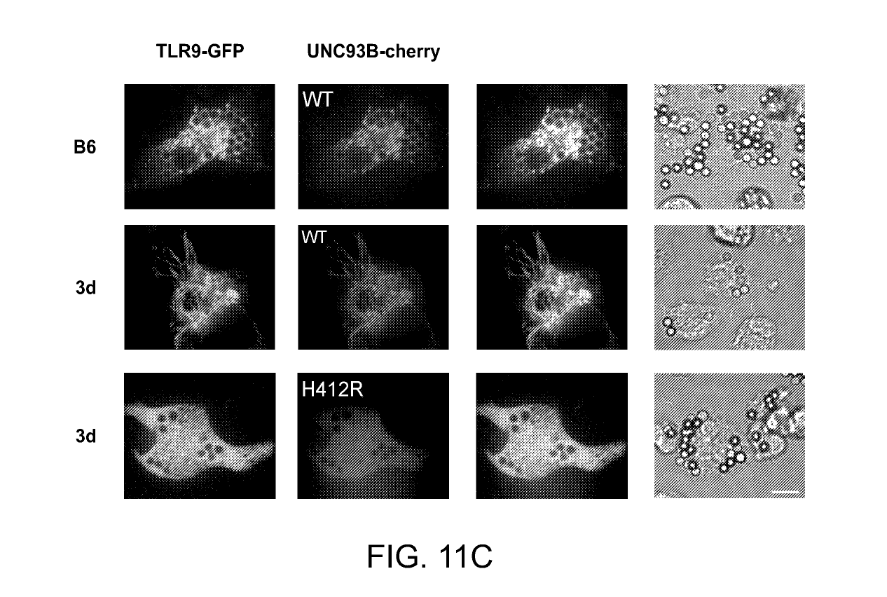

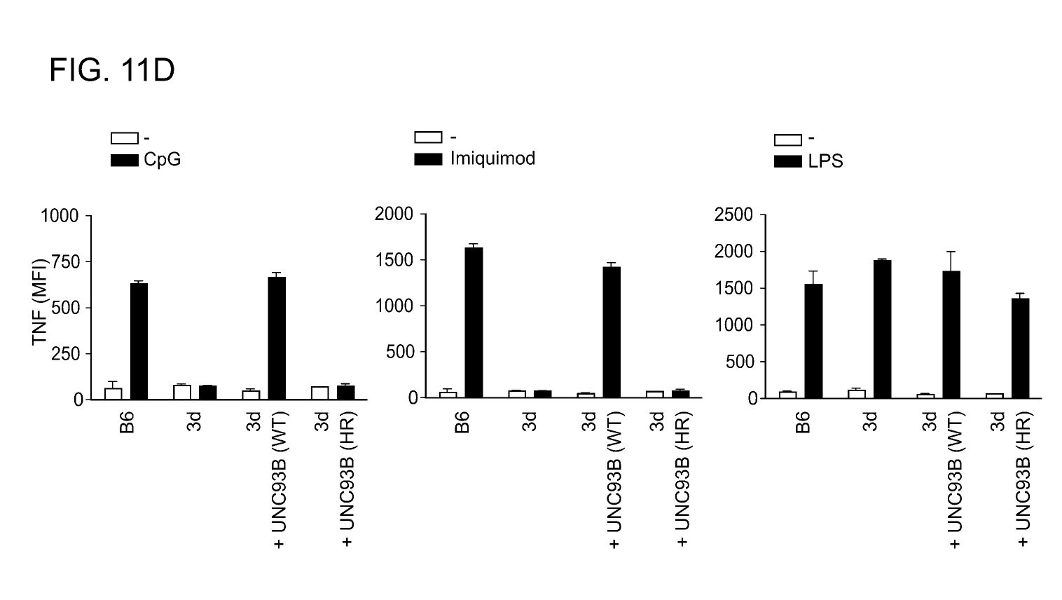

FIG. 11. Expression of wild type UNC93B in 3d cells rescues defects of TLR trafficking and signaling. a, TLR9-GFP and UNC93B-cherry (wild type or H412R mutant) were coexpressed in B6 or 3d BM-DCs. After stimulation with 1 μM CpG for 2 h, live cells were imaged. b and c, TLR9-GFP alone (b) or TLR9-GFP and UNC93B-cherry (wild type or H412R mutant) together (c) were expressed in B6 or 3d BM-DCs. Cells were incubated with 3 μm polystyrene beads for 1 h and imaged. d, BM-DCs (B6 or 3d) expressing GFP alone without exogenous UNC93B expression or 3d BM-DCs expressing either wild type or mutant UNC93B-GFP were stimulated with 1 μM CpG (left panel), 100 nM imiquimod (middle panel) or 1 μg/ml LPS (right panel) for 4 h in the presence of 10 μg/ml brefeldin A. Cells were fixed and stained for intracellular TNF. TNF levels in GFP-positive cells were measured by flow cytometry. MFI; mean fluorescence intensity, Scale bars; 10 μm

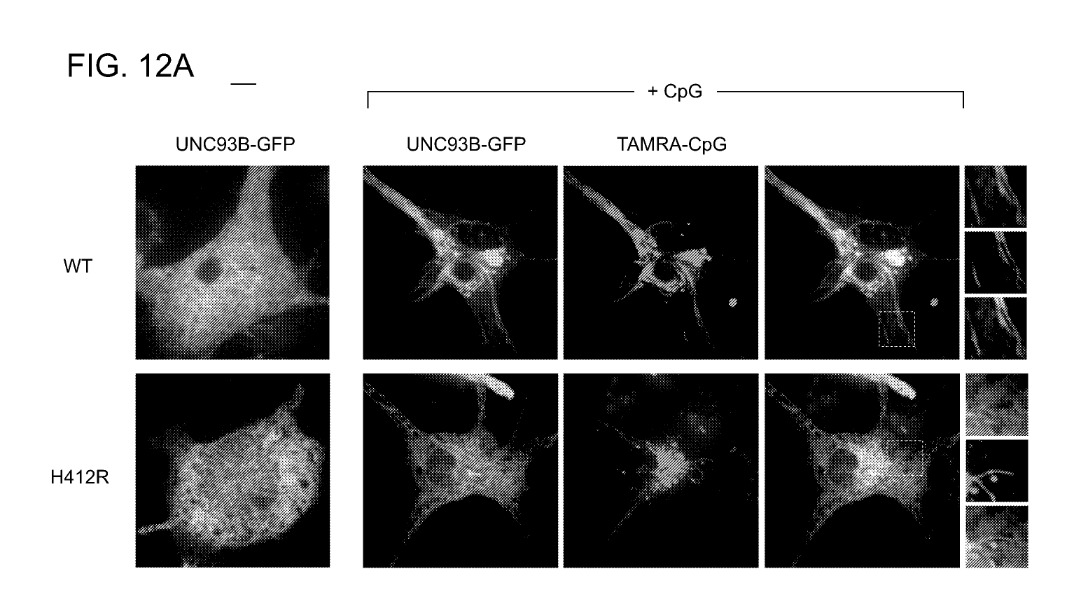

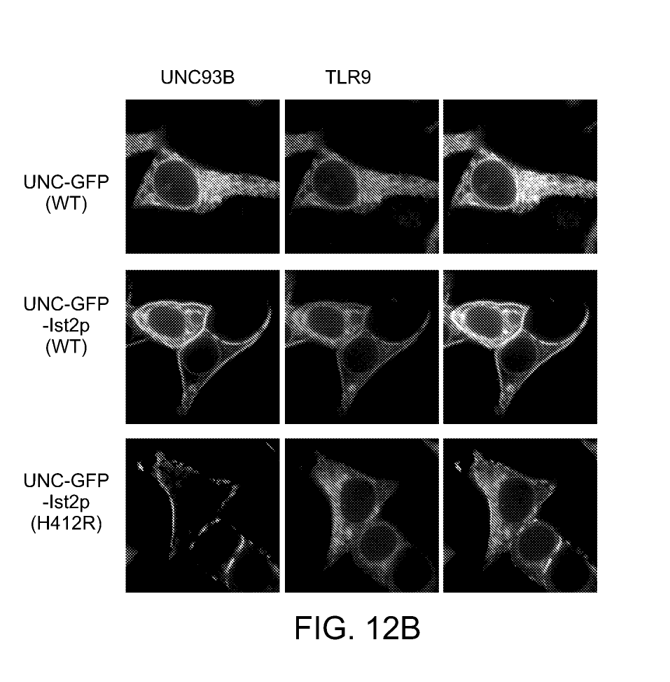

FIG. 12. Wild type UNC93B delivers the nucleotide-sensing TLRs to endolysosomes. a, Wild type or H412R mutant UNC93B-GFP was expressed in B6 and 3d BM-DCs, respectively. Cells were either left untreated (−) or incubated with 1 μM TAMRA-labeled CpG for 15 min, washed and imaged after an additional 2 h incubation (+CpG). b, UNC93B-GFP or UNC93B-GFP-Ist2p (wild type or H412R mutant) was coexpressed with TLR9-cherry in HEK-293T cells. Cells were imaged 24 h post-transfection.

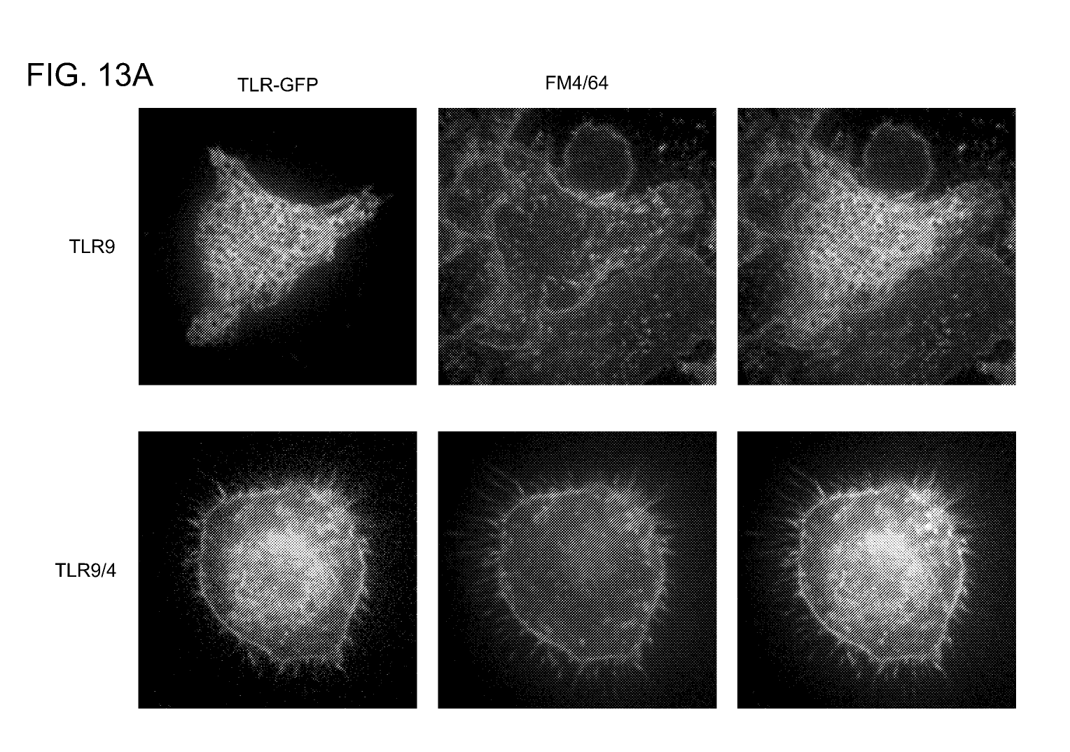

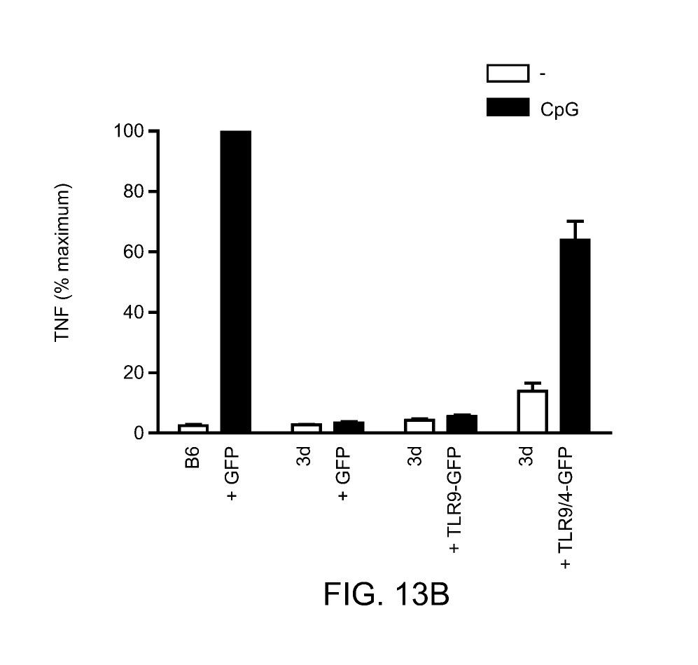

FIG. 13. UNC93B is dispensable for ligand recognition and signal initiation by TLR. a, TLR9-GFP or TLR9/4-GFP was expressed in 3d BM-DCs. Live cells were imaged after the plasma membrane was stained with the lipophilic dye FM4/64. b, BM-DCs (B6 or 3d) expressing GFP alone without exogenous TLR expression or 3d BM-DCs expressing either TLR9-GFP or TLR9/4-GFP were stimulated with 1 μM CpG for 4 h in the presence of 10 μg/ml brefeldin A. Cells were fixed and stained for intracellular TNF. TNF levels in GFP-positive cells were measured by flow cytometry. The data (mean±SEM) are generated from four independent experiments and the levels of TNF are represented as the percentage of TNF level detected in B6 cells.

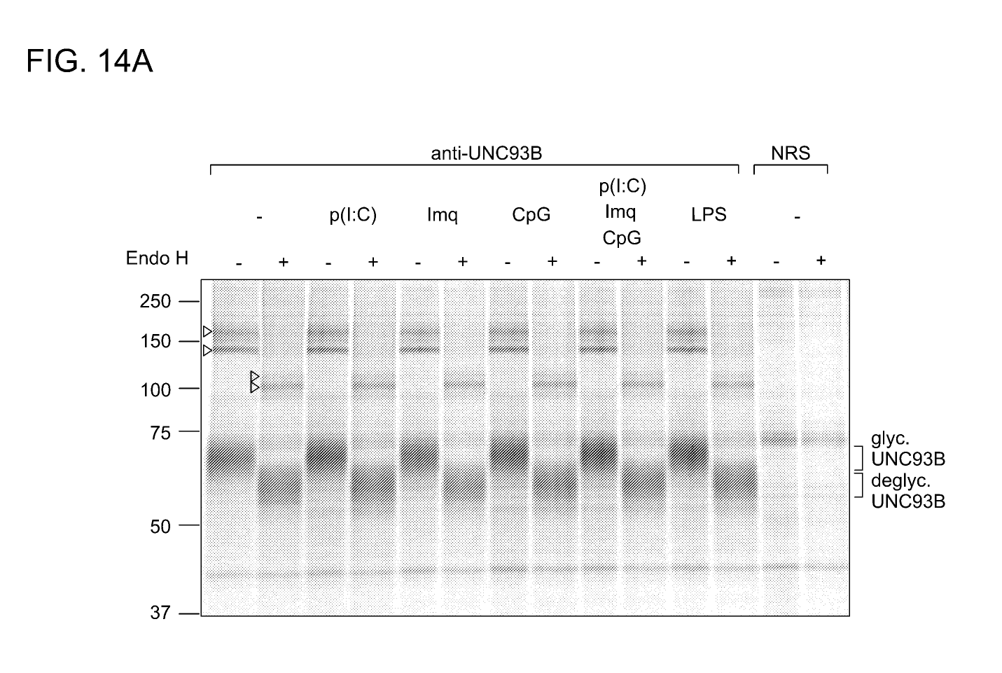

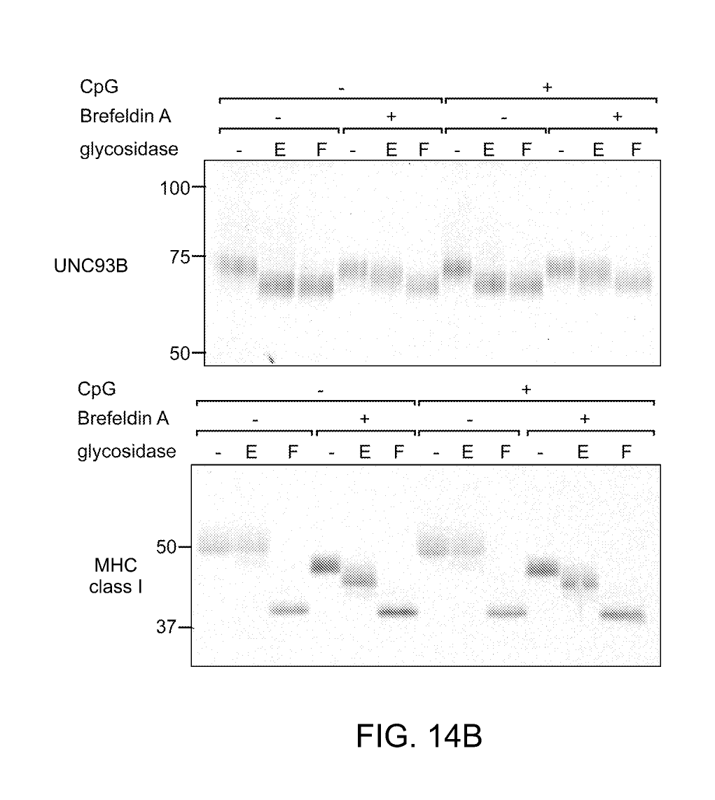

FIG. 14. The interaction between UNC93B and TLRs maintains in dendritic cells stimulated with TLR agonists. (a) BM-DCs were metabolically labeled with ³⁵S-methionine/cysteine and stimulated with TLR ligands for 1 h (LPS: 1 μg/ml, poly (I:C): 50 μg/ml, Imiquimod: 2.5 CpG DNA: 2.5 μM). Endogenous UNC93B and TLRs were immunoprecipitated with an anti-UNC93B antibody, digested with endoglycosidase H (Endo H), subjected to 10% SDS-PAGE and visualized by fluorography. TLRs coimmunoprecipitated with UNC93B are denoted by open triangles. Interaction between TLRs and UNC93B was maintained even after TLR stimulation with various agonists. Neither UNC93B nor TLRs acquire resistance to Endo H digestion, which indicates that they are not exposed to carbohydrate modifying enzymes in the Golgi apparatus. NRS; normal rabbit serum (b) RAW macrophages stably expressing wild type UNC93B-HA were exposed to Brefeldin A (10 μg/ml) for 2 h, metabolically labeled with ³⁵S-methionine/cysteine for 30 min (pulse), incubated in normal medium for 2 h (chase) followed by 2 h stimulation with CpG (+CpG) or were left unstimulated (−CpG). All incubations (pulse, chase, CpG stimulation) were done in the continuous presence of Brefeldin A. As a control, cells were not treated with Brefeldin A (−BA). Cells were lysed in RIPA buffer and UNC93B-HA and Class I MHC products were recovered from cell lysates by immunoprecipitation with an anti-HA (upper panel) or Class I antibody (lower panel), respectively. Re-immunoprecipitation was carried out with an anti-UNC93B antibody (upper panel) or Class I antibody (lower panel) after denaturation of the immunoprecipitates. Recovered UNC93B-HA and Class I MHC were subjected to digestion with glycosidases Endo H (E) or PNGase F (F) or not digested (−). Normally, UNC93B retains full sensitivity to Endo H digestion, irrespective of stimulation with TLR agonists, whereas surface disposed Class I MHC products acquire EndoH resistance. In the presence of Brefeldin A, which disrupts the Golgi apparatus and relocalizes Golgi-resident proteins to the ER, both Class I MHC and UNC93B acquire partially EndoH resistant hybrid type oligosaccharides.

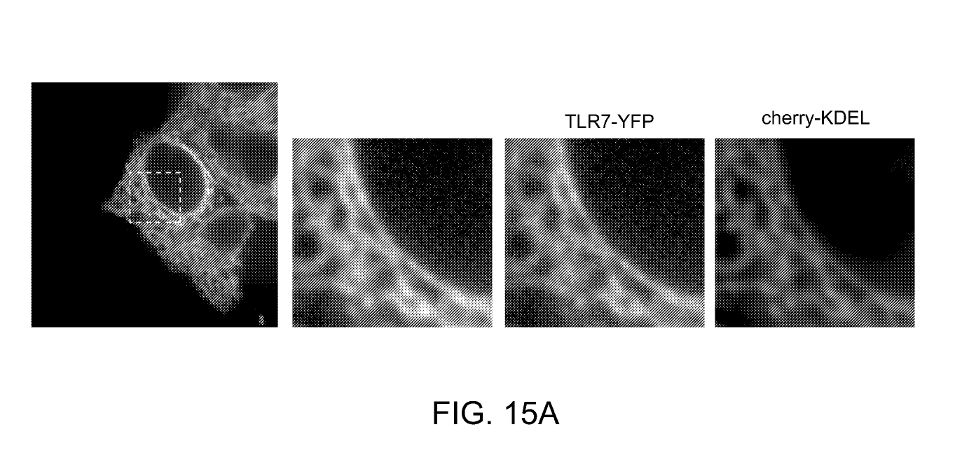

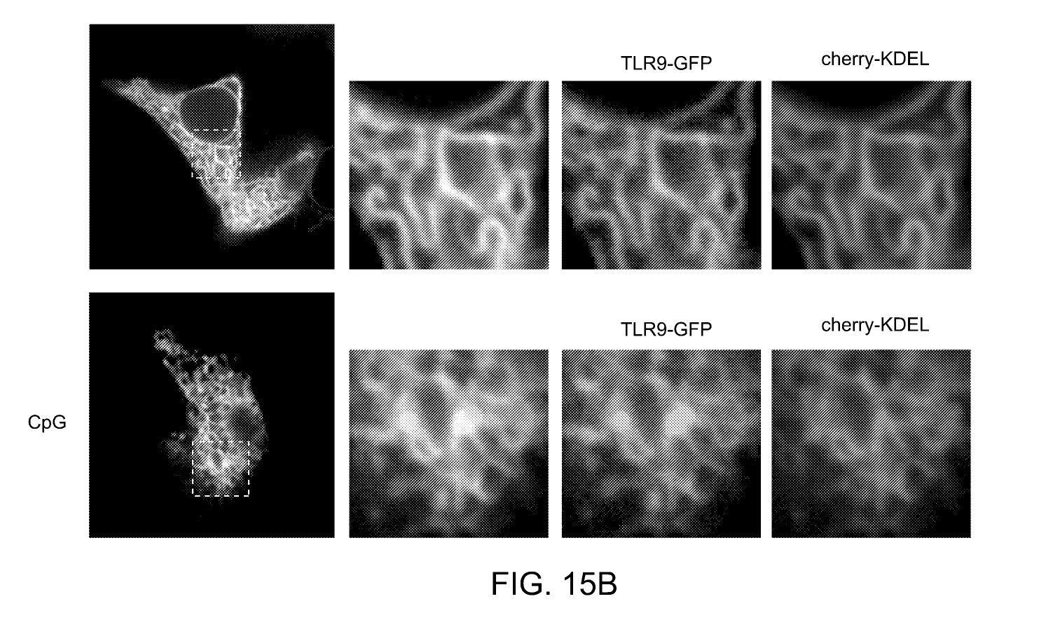

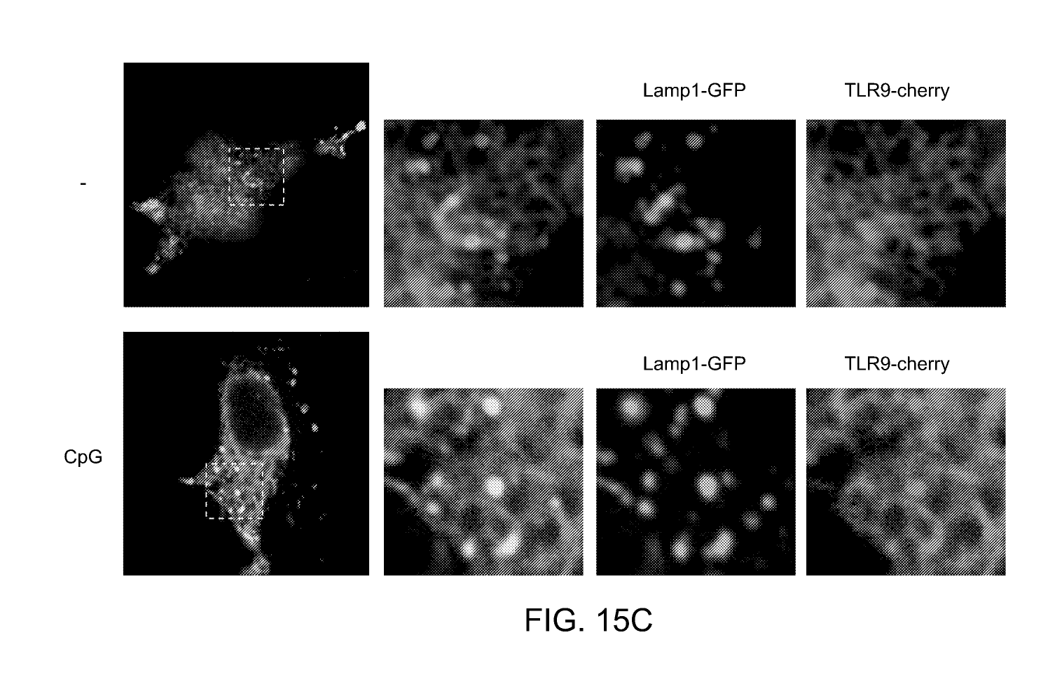

FIG. 15. TLR7 and TLR9 are localized in the ER of unstimulated cells and translocate to endosomes upon stimulation of cells. TLR7-YFP (a), TLR9-GFP (b) or TLR9-cherry (c) were coexpressed with either cherry-KDEL (a,b) or Lamp1-GFP (c) in HEK-293T cells by transient transfection. After 48 h, live cells were imaged either unstimulated (−) or stimulated with 1 μM CpG for 2 h (CpG). Both TLR7-YFP and TLR9-GFP colocalize with the ER marker cherry-KDEL in unstimulated cells. In CpG-stimulated cells, a fraction of TLR9-GFP does not colocalize with cherry-KDEL, suggesting that they are exported from the ER. Accordingly, a fraction of TLR9-cherry colocalizes with the lysosomal marker Lamp1-GFP in CpG-stimulated cells.

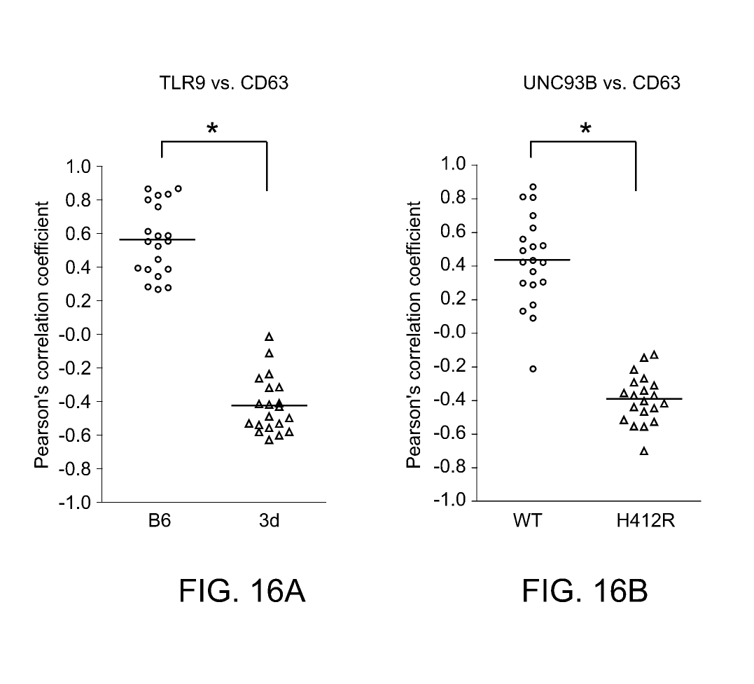

FIG. 16. TLR9 and UNC93B colocalize with CD63 in CpG-stimulated wild type cells. (a) TLR9-GFP was coexpressed with CD63-cherry in either B6 or 3d BM-DCs. (b) Wild type and mutant (H412R) UNC93B-GFP were coexpressed with CD63-cherry in B6 and 3d BM-DCs, respectively. After stimulation with 1 μM CpG for 2 h, twenty live cells from each group were randomly chosen and imaged for both GFP and cherry signals. Colocalization of GFP and cherry signals was quantified by using the MetaMorph colocalization analysis tools. Mean±SEM of Pearson's correlation coefficients are 0.56±0.05 for TLR9 vs. CD63 in B6 cells, −0.43±0.04 for TLR9 vs. CD63 in 3d cells, 0.43±0.06 for wild type UNC93B vs. CD63 in wild type cells and −0.39±0.03 for mutant UNC93B vs. CD63 in 3d cells. *; P<0.001, t-test

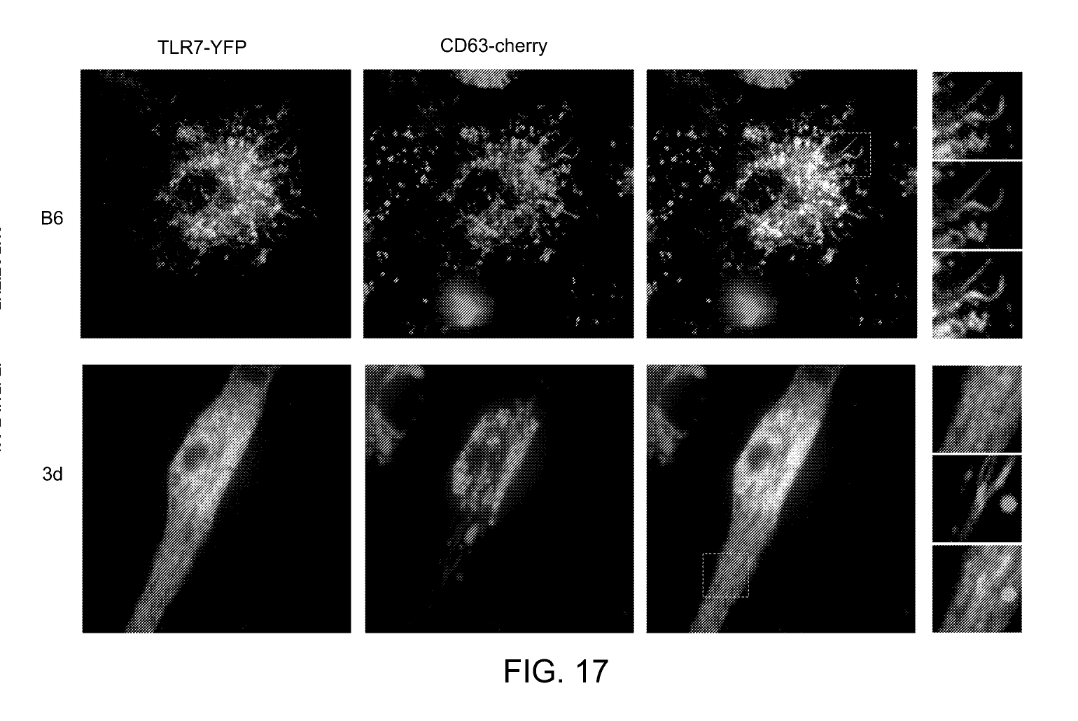

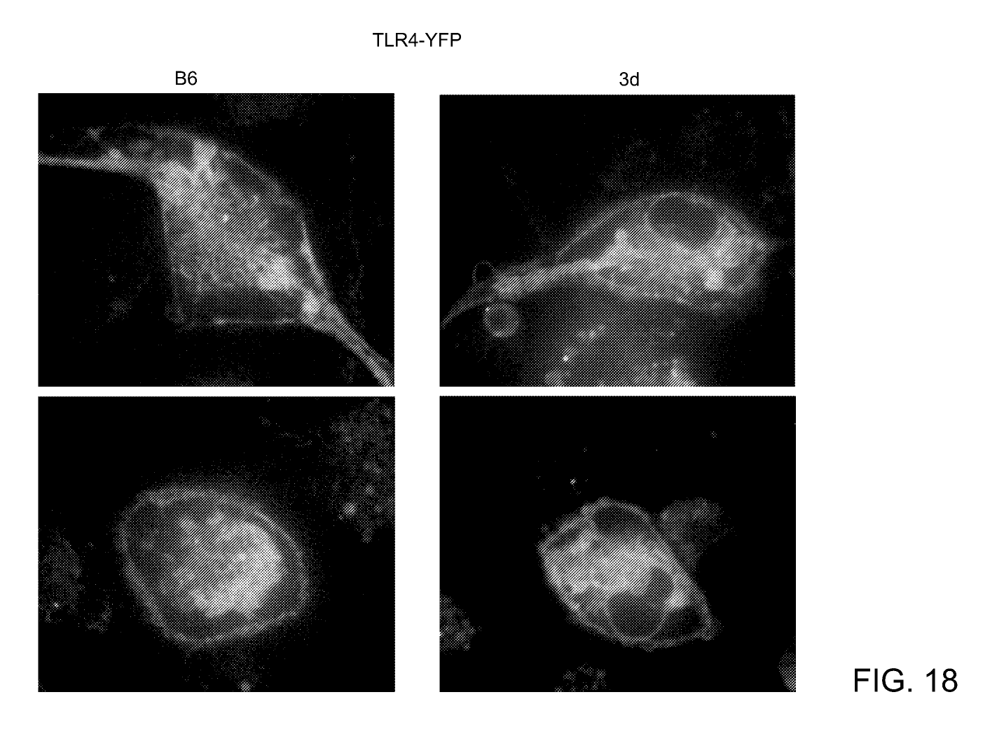

FIG. 17. TLR7 fails to translocate to endolysosomes upon stimulation of 3d BM-DCs. TLR7-YFP was coexpressed with CD63-cherry in either B6 or 3d BM-DCs and live cells were imaged after stimulation with 1 μM imiquimod for 2 h. In 3d cells, TLR7 remains in the ER, whereas it colocalizes with CD63 in endolysosomes of B6 cells.

FIG. 18. Localization of TLR4 is normal in 3d cells. TLR4-YFP was expressed in either B6 or 3d BM-DCs and live cells were imaged after stimulation with 1 μg/ml LPS for 2 h. Shown are two exemplary BM-DCs from each mouse. In both B6 and 3d cells, TLR4 localizes to the cell surface and inside the cells.

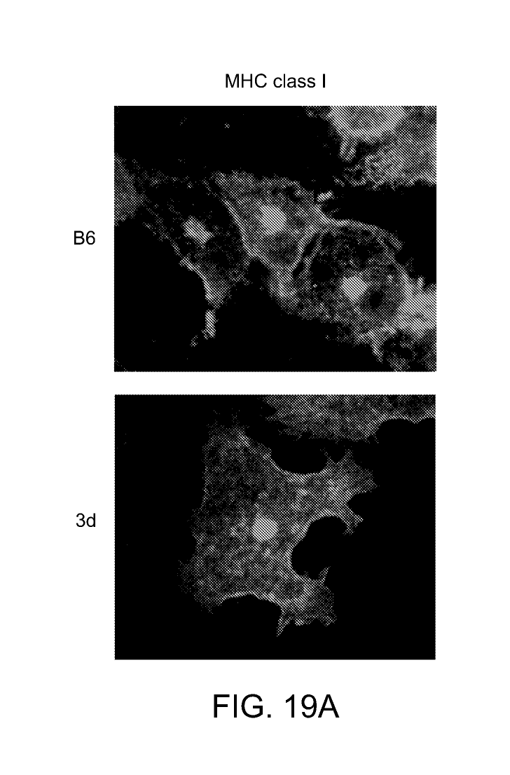

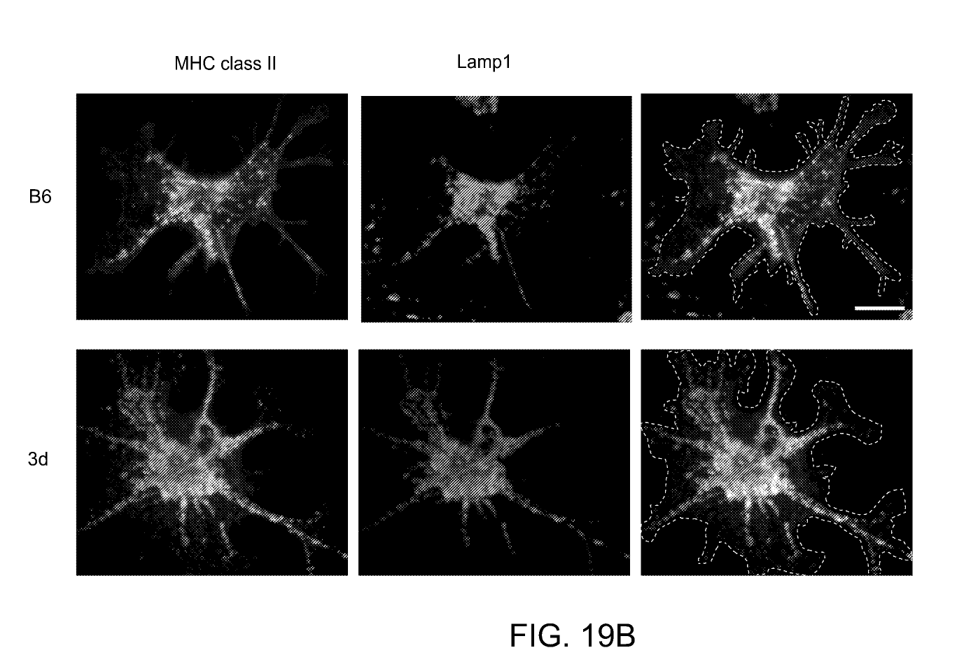

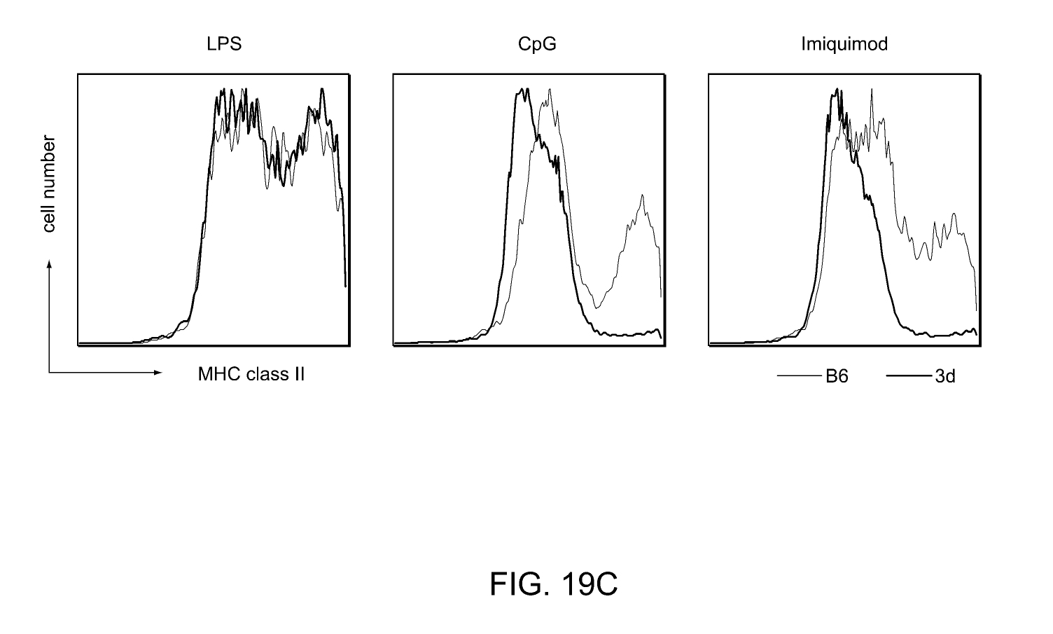

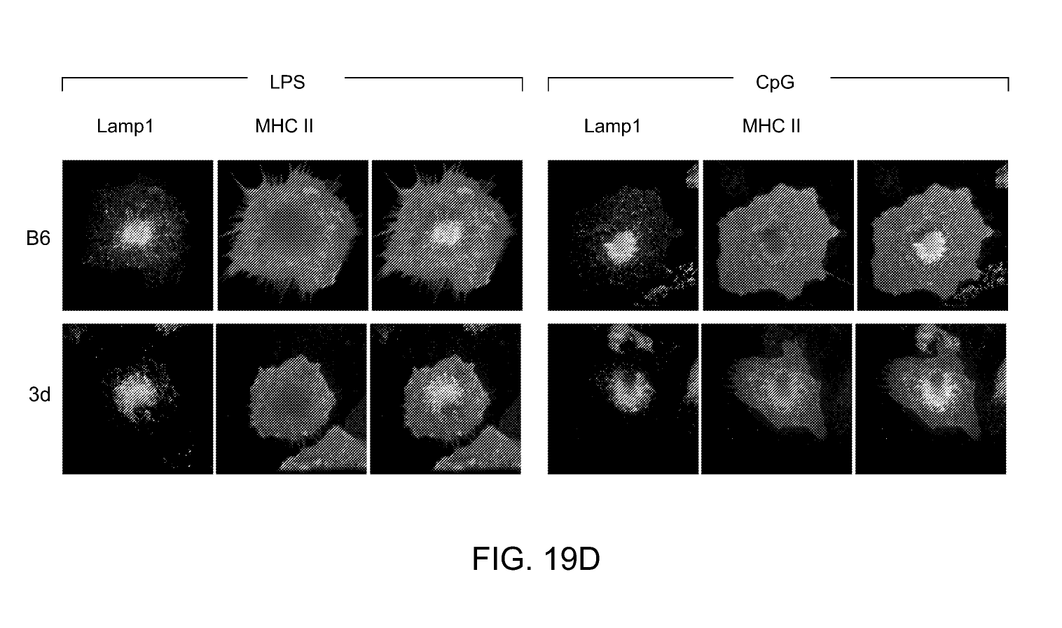

FIG. 19. Localization of class I and class II MHC molecules is normal in 3d cells. (a) BM-DCs from B6 and 3d mice were fixed, permeabilized and stained for MHC class I. In both wild type and 3d dendritic cells, Class I MHC is present at the cell surface and in the perinuclear region. (b) BM-DCs from B6 and 3d mice were fixed, permeablized and stained for MHC class II (green) and Lamp1 (red). In both wild type and 3d dendritic cells, class II MHC molecules reside in Lamp1-positive endosomes. White dashed lines mark the cell boundary. (c) Wild type (red line) or 3d (blue line) BM-DCs were stimulated with 1 μg/ml LPS (left), 1 μM CpG (middle) or 1 μM imiquimod (right) for 16 h and the level of MHC class II on the cell surface was measured by staining with PE-conjugated anti-I-A^{b} antibody and flow cytometry. LPS upregulated the surface level of MHC class II equally in B6 and 3d cells. In contrast, CpG and imiquimod failed to increase the MHC class II level in 3d cells. (d) B6 or 3d BM-DCs were stimulated with 1 μg/ml LPS or 1 μM CpG for 16 h, fixed, permeabilized and stained for MHC class II and Lamp1. In both B6 and 3d cells, MHC class II molecules were mainly found at the cell surface upon LPS stimulation. Therefore, translocation of Class II MHC to the plasma membrane does not require UNC93B. When cells were stimulated with CpG, less MHC class II molecules were found at the cell surface of 3d cells. Scale bars; 10 μm

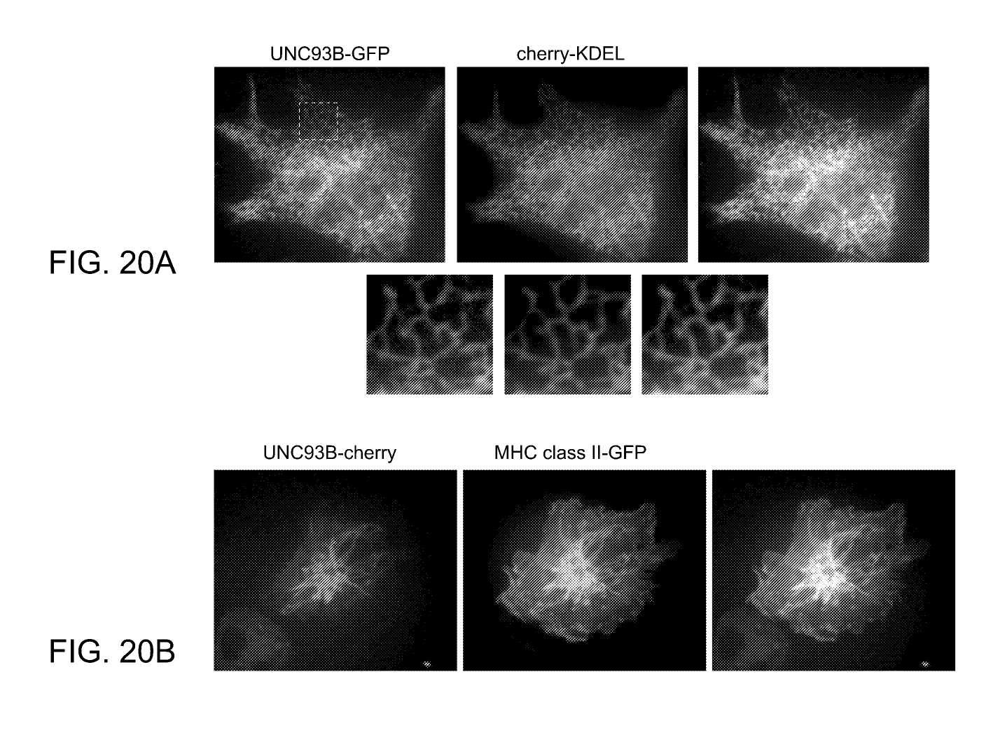

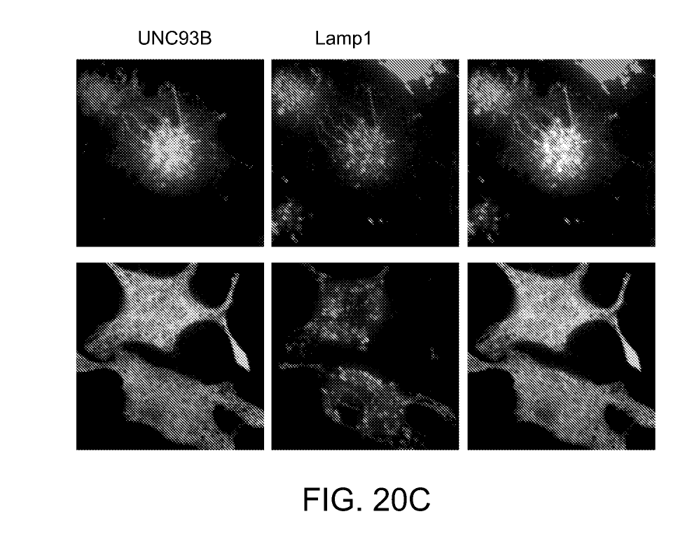

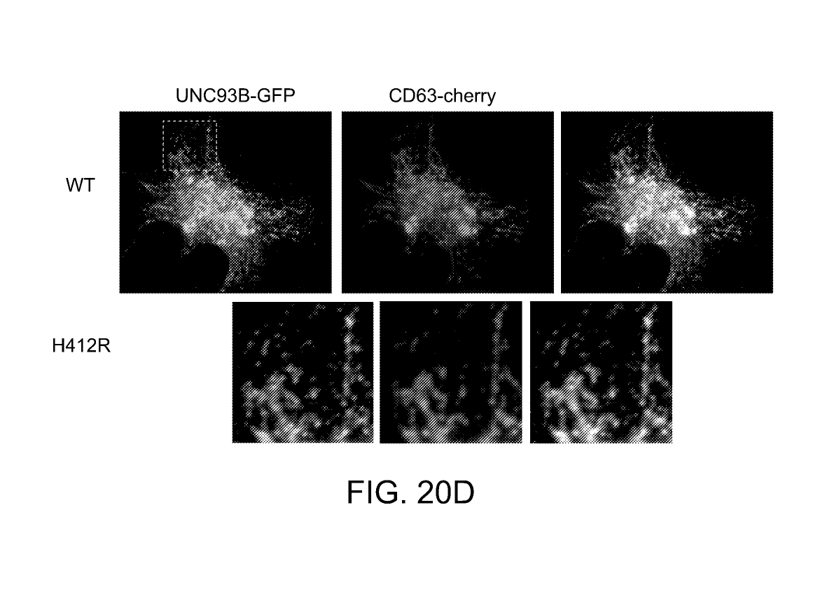

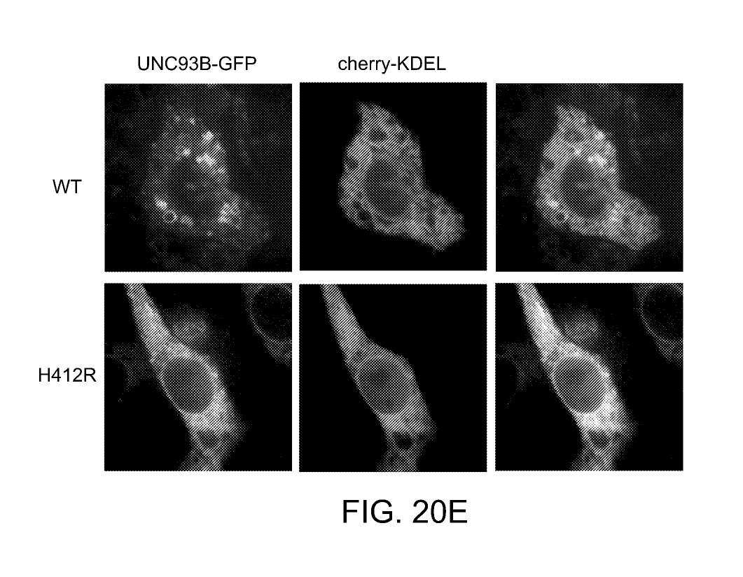

FIG. 20. Wild type but not mutant UNC93B translocates to endolysosomes in cells stimulated with CpG. (a) Wild type UNC93B-GFP was coexpressed with cherry-KDEL in HEK-293T cells by transient transfection. Cells were imaged at 24 h post-transfection. UNC93B-GFP colocalizes with the ER marker cherry-KDEL. (b) Wild type UNC93B-cherry was expressed in BM-DCs from MHC class II-GFP knock-in mice in which the endogenous I-A^{b}β molecule was substituted with GFP-tagged I-A^{b}β (Boes, M. et al. T-cell engagement of dendritic cells rapidly rearranges MHC class II transport. Nature 418, 983-8 (2002)). Cells were imaged after stimulation with 1 μM CpG for 2 h. UNC93B colocalizes with MHC class II in endolysosomes. (c) BM-DCs from B6 mice were stimulated with 1 μM CpG for 2 h, fixed, permeabilized and stained with affinity purified rabbit polyclonal anti-UNC93B (green) and monoclonal Lamp1 (red) antibodies. Endogenous wild type UNC93B colocalizes with Lamp1. (d) Wild type and H412R mutant UNC93B-GFP proteins were coexpressed with CD63-cherry in B6 and 3d BM-DCs, respectively. Live cells were imaged after stimulation with 1 μM CpG for 2 h. Only wild type UNC93B colocalized with CD63 in endolysosomes. (e) Wild type or H412R mutant UNC93B-GFP proteins were stably coexpressed with cherry-KDEL in RAW macrophages. Live cells were imaged after stimulation with 1 μM CpG for 2 h. A large fraction of wild type UNC93B-GFP does not colocalize with cherry-KDEL, indicating that they are exported from the ER. In contrast, mutant UNC93B-GFP remained colocalized with cherry-KDEL.

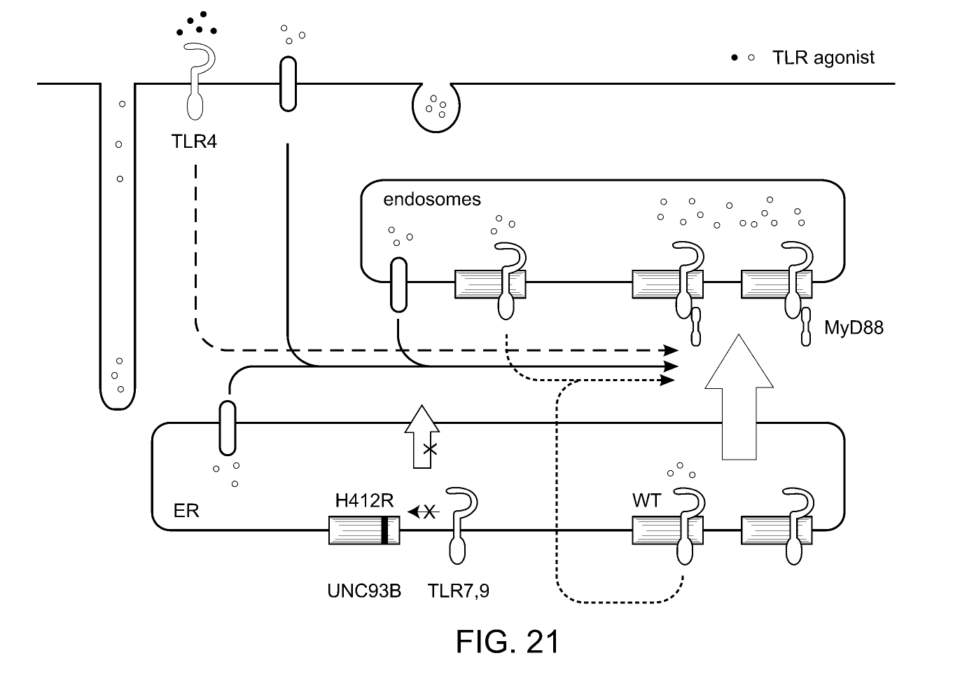

FIG. 21. Signals that promote transport of UNC93B/TLR complex may originate from multiple receptors. Wild type but not mutant UNC93B binds to the nucleotide sensing TLRs in the ER and delivers them to endosomes. Signals that promote transport of UNC93B/TLR complex may originate from receptors that include the TLRs themselves, but possibly also receptors that remain to be identified. These receptors could be located at the surface, in endosomes or even in the ER. Blue arrows; Small amounts of agonist for TLR7 and TLR9 may reach the ER—via a route known to delivers some bacterial toxins and viruses (Spooner, R. A., Smith, D. C., Easton, A. J., Roberts, L. M. & Lord, J. M. Retrograde transport pathways utilised by viruses and protein toxins. Virol J 3, 26 (2006))—and thus stimulate the receptors to induce transport of the remainder of the ER-resident TLRs to endosomes. Alternatively, a minor fraction of TLR7 and TLR9 may constitutively reside in endosomes and sense the internalized agonists. Activation of such TLRs leads to recruitment of the ER-resident TLRs to endosomes. Yellow arrows; Activation of TLR4 by LPS also results in translocation of UCN93B/TLR complexes from the ER to endosomes. Pink arrows; The as yet unidentified protein (depicted as a purple rod) may recognize TLR agonists and promote transport of UCN93B/TLR complexes to endosomes. In endosomes, TLR7 and TLR9 recognize the bulk of internalized agonists and initiate signaling via recruitment of MyD88 adaptor molecules.

### DETAILED DESCRIPTION OF CERTAIN EMBODIMENTS OF THE INVENTION

### I. Definitions

The following definitions are provided for the convenience of the reader. It is not intended that these definitions conflict with art-accepted definitions of any of these terms.

“Adapted to specifically identify an agent that modulates the physical interaction between an UNC93B polypeptide and the UNC93B-dependent TLR polypeptide”, in reference to an assay, means that the assay can be used and includes steps sufficient to distinguish an agent that modulates the physical interaction between an UNC93B polypeptide and the UNC93B-dependent TLR polypeptide from other agents including, but not limited to, agents that modulate signaling by the UNC93B-dependent TLR by means other than by modulating the interaction. An assay composition is “adapted to specifically identify an agent that modulates the physical interaction between an UNC93B polypeptide and the UNC93B-dependent TLR polypeptide” if it is usable in such an assay, with the proviso that compositions known in the prior art including, but not limited to, certain compositions wherein the UNC93B polypeptide, the UNC93B-dependent TLR polypeptide, or in some embodiments both (i) is modified from its naturally occurring form such as by addition or incorporation of a detectable moiety, epitope tag, etc. and/or (ii) is provided as a fusion protein or attached to a solid support are explicitly excluded.

“Antibody” as used herein refers to immunoglobulin molecules or portions thereof capable of specifically binding an antigen, and includes polyclonal antibodies or fragments thereof and monoclonal antibodies or fragments thereof, provided that such fragments contain a portion capable of binding to an antigen. Antibodies or purified fragments having an antigen binding region, including fragments such as Fv, Fab′, F(ab′)₂, Fab fragments, single chain antibodies (which include the variable regions of the heavy and light chains of an immunoglobulin, linked together with a short (usually serine, glycine) linker, chimeric or humanized antibodies, and complementarily determining regions (CDR) may be prepared by conventional procedures.

“Approximately” or “about” generally includes numbers that fall within a range of 1% or in some embodiments 5% of a number in either direction (greater than or less than the number) unless otherwise stated or otherwise evident from the context (except where such number would impermissibly exceed 100% of a possible value).

“Assay composition” as used herein refers to a composition comprising at least two components (e.g., an UNC93B and an UNC93B-dependent TLR polypeptide) suitable for performing an assay of the present invention. The components could be, e.g., isolated, purified, or within cells. “Assay system” as used herein refers to an assay composition and, optionally, one or more additional components or apparatus necessary or useful for performing the assay including, but not limited to, a vessel in which the assay composition is located, an enzyme substrate, detection apparatus, reference sample, etc. Optionally the assay composition comprises a suitable liquid medium for performing the assay. Optionally the assay composition includes buffers, salts, stabilizers such as protease inhibitors or anti-oxidants, a candidate agent, etc.

“Autoantigen” or “self antigen” refers to an endogenous substance found within a subject (e.g., a mammal) and normally recognized by the immune system as self, but which is, in particular subjects such as those suffering from an autoimmune disease, aberrantly recognized as foreign by the subject. In other words, despite being a normal tissue constituent, the antigen is not recognized as part of the subject by the immune system cells or antibodies of the subject and is inappropriately attacked by the immune system as though it were a foreign substance. Aberrant recognition of the antigen may arise as a result of the antigen having been modified in the subject and/or as a result of the subject having been exposed to an exogenous antigen that comprises one or more epitopes shared by the endogenous substance. An autoantigen according to the invention may include an epitope shared with or derived from a foreign antigen. Examples of autoantigens include, in various conditions, single or double stranded DNA or RNA and various proteins. Specific examples include glutamate decarboxylase, insulin, myelin basic protein, type II collagen, nicotinic acetylcholine receptor, thyroglobulin, Jo-1 antigen La (SSB) antigen, PCNA antigen, Ribosomal P antigen, RNP A, RNP/Sm, Ro52, Sc170, and Islet cell antigen 69.

“Autoimmune” refers to a disease caused at least in part by an inability of the immune system to distinguish foreign molecules from self molecules, and a loss of immunological tolerance to self antigens, causing the immune system, or effector molecules thereof such as antibodies or complement, to inappropriately attack and damage the cells, tissues, or organs that with which the self antigens are associated, e.g., that express and/or display the self antigens.

“Candidate modulator” or “candidate agent” refers to an agent to be evaluated or being evaluated for its effect on TLR signaling and/or on the interaction of an UNC93B polypeptide and an UNC93B-dependent TLR. Exemplary sources and types of candidate agents useful according to the invention are described below.

“Cell line” refers to a population of largely or substantially identical cells that has been derived from a single ancestor cell or from a defined population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

“Characterized by aberrant or abnormal function of the immune system” refers to a condition of a subject in which there is inappropriate or undesirably excessive immune system activity or in which immune system activity is inappropriately or undesirably reduced or diminished. It will be appreciated that “inappropriate or undesirably” refers to a condition that is inappropriate or undesired under the particular circumstances and may reflect a response that is considered “normal” but that nonetheless has pathogenic consequences for the subject. Diseases and clinical conditions involving or caused at least in part by immune system activity or lack thereof are encompassed. In various embodiments, the following general types of condition are considered to be characterized by aberrant or abnormal function of the immune system: autoimmune diseases, acute or chronic inflammatory diseases, and increased as susceptibility to infection.

“Cytokine” refers to a variety of soluble polypeptides produced by animal (e.g., mammalian) cells that act as humoral regulators and signaling molecules at low, e.g., micro- to picomolar concentrations and can, under either normal or pathological conditions, modulate the functional activities of individual cells and tissues following their binding to a cellular receptor. Cascades of intracellular signalling triggered by such binding may include the upregulation and/or downregulation of various genes, including transcription factors, in turn resulting in the production of other cytokines, an increase in the number of surface receptors for other molecules, etc. Cytokines have been classified as pro-inflammatory or anti-inflammatory. Pro-inflammatory cytokine are typically capable of directly or indirectly causing any of the following physiological reactions associated with inflammation: vasodilation, hyperemia, increased permeability of vessels with associated edema, migration and accumulation of immune system cells such as granulocytes, lymphocytes, and mononuclear phagocytes. Exemplary cytokines include IL-6, IL-12, IL-18, tumor necrosis factor-α (TNF-α), IFNs α, β, and γ, MIP-1α, MCP-1, RANTES, etc.

“Expression” refers to the cellular processes involved in producing RNA and proteins and as appropriate, secreting proteins, including where applicable, but not limited to, for example, transcription, translation, folding and post translational modification and processing.

A “genetically modified” or “engineered” cell as used herein refers to a cell into which an exogenous nucleic acid has been introduced (or a descendant of such a cell). The nucleic acid may have subsequently been removed or excised from the genome, provided that such removal or excision results in a detectable alteration in the cell relative to an unmodified but otherwise equivalent cell. In some embodiments, the cell is stably transformed by the exogenous nucleic acid. In some embodiments, at least a portion of the exogenous nucleic acid integrates into the genome. The exogenous nucleic acid could be introduced in a vector (e.g., plasmid, viral vector, etc.). In some embodiments, the exogenous nucleic acid is expressed in the cell. In some embodiments, the exogenous nucleic acid is or encodes an agent that interferes with expression of an endogenous polypeptide via a sequence-specific gene-silencing mechanism such as RNA interference (RNAi).

“Identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the World Wide Web at ncbi.nlm.nih.gov for these programs. In a specific embodiment, percent identity is calculated using BLAST2 with default parameters as provided by the NCBI as of the filing date hereof

“Immune response” refers to the collective activity of various components of the immune system against foreign antigens such as those found on infectious agents or substances that enter or contact the body from the external environment or, in the cases of autoimmune disease, pathological inflammation, and certain tumors, against self antigens that are perceived as foreign. In certain embodiments, an immune response involves the action of lymphocytes, antigen presenting cells, natural killer cells, phagocytic cells such as macrophages, granulocytes, and/or soluble macromolecules produced by certain of the above cells and/or the liver (e.g., antibodies, cytokines, and complement) and results, for example, in damage to, destruction of, and/or elimination from the body of the substance against which the response is directed. The immune response has been broadly divided into “adaptive” and “innate” immune responses. The former includes those aspects of the immune response that provide the vertebrate immune system with the ability to recognize and remember specific antigens (to generate immunity), and in many instances to mount a more rapid and stronger attack upon subsequent encounters with a specific antigen. The latter includes those aspects of the immune response that provide nonspecific defense against foreign antigens but do not confer long-lasting immunity or immunological memory. “Inflammation” is an example of an innate immune response that can occur not only in response to foreign or self antigens but also in response to tissue damage or irritation caused by trauma or burn, chemical agents (e.g., toxins), environmental conditions such as heat, pressure, etc. Inflammation is classically considered to be characterised by the following quintet: redness, heat, swelling, pain, and dysfunction of the organs involved, although one of skill in the art will recognize that one or more of these may be absent depending on the site and cause of the inflammation. Inflammation initially involves vascular dilation and increased permeability and the movement of white blood cells and fluids from blood vessels into the affected tissue, associated with release of a variety of inflammatory mediators such as histamine, bradykinin, leukotrienes, and prostaglandins. Inflammation can be acute or chronic and can be systemic or local. Acute inflammation as used herein includes those responses that occur within the first 1-2 weeks following an inciting stimulus. Chronic inflammation as used herein refers to inflammation that persists at a detectable level, albeit possibly at a low level, over at least 30 days, typically 1-12 months, 1-5 years, or longer, often leading to tissue infiltration with macrophages and fibroblast cells and formation of new connective tissue.

“Immune system activity” refers to any one or more activities of the immune system and includes the in vitro or in vivo response of immune system cells to any of a variety of stimuli (e.g., antigens, cytokines, other cells), causing biochemical events within the cells that lead to immune cell migration, killing of target cells, phagocytosis, antigen presentation, production of antibodies and/or other soluble effectors such as cytokines, upregulation of cell surface receptors, proliferation of immune system cells, etc.

“Infectious agent” refers to viruses, bacteria, fungi, and parasites capable of colonizing and, typically, multiplying in a host organism leading, in some instances, to pathological effects.

The terms “interact” or “physically interact” means that two molecular species physically associate with each other. The association that is characterized as an interaction can involve, e.g., charge-charge interactions, charge-dipole interactions, dipole-dipole interactions, van der Waals forces, hydrogen bonding and/or hydrophobic forces.

The term “introducing a polynucleotide into a cell” refers to the process whereby a recombinant polynucleotide is put into a cell. Methods for introducing a polynucleotide into a cell will vary with the nature of the cell and the nature of the chosen vector. One of skill in the art may readily select and employ a known method appropriate for a given cell type and vector.

“Linker” refers to an entity that joins two moieties that are not found joined together in nature. “Linker” or “Linker region” can refer, for example, to a sequence of peptide bonded amino acids that joins or links by peptide bonds two amino acid sequences or polypeptide domains that are not joined by peptide bonds in nature.

The term “modulation of interaction” or “change in interaction” refers to an increase or decrease in the level of interaction detected between members of a given pair of molecular species. As used herein, the level of interaction is considered increased if the detected interaction goes up by at least 10%, e.g., by 20%, 35%, 50%, 75%, or more, up to and including 2-fold, 5-fold, 10-fold, 20-fold, 50-fold or more relative to a standard. As used herein, the level of interaction is considered decreased if the detected interaction goes down by at least 10%, e.g., by 20%, 35%, 50%, 75%, 90%, 95%, 98%, 99% or more, up to and including 100% (no interaction) relative to a standard.

“Modulate” means to cause or facilitate an alteration or modification, e.g., an increase or decrease, in a process or phenomenon of interest. A “modulator” is an agent that causes or facilitates an alteration or modification, e.g., an increase or decrease, in a process or phenomenon of interest. For example, “modulate” can refer to an ability to inhibit (i.e. reduce and/or down-regulate) or activate (increase, enhance, and/or upregulate) a process or phenomenon of interest. In certain embodiments “modulating TLR signaling” refers to inhibiting at least one indicator of TLR signaling by at least about 10%, 25%, 50%, 75%, or 100%. In certain embodiments, “modulating TLR signaling” refers to causing an increase in at least one indicator of TLR signaling by at least about 10%, 25%, 50%, 75%, 100%, 200%, 500%, or 1000-5000%, or more. In one embodiment, the indicator is release of a pro-inflammatory cytokine, e.g., TNF-α. An “immunomodulator” is an agent capable of altering one or more activities of the immune system when contacted with immune system cells and/or administered to a subject. In certain embodiments, “modulating immune system activity” refers to inhibiting at least one indicator of immune system activity by at least about 10%, 25%, 50%, 75%, or 100%. In certain embodiments, “modulating the immune system” refers to signaling refers to causing an increase in at least one indicator of immune system activity by at least about 10%, 25%, 50%, 75%, 100%, 200%, 500%, or 1000-5000%, or more. In one embodiment, the indicator is release of a pro-inflammatory cytokine, e.g., TNF-α. In one embodiment, the indicator is production of an antibody. In one embodiment, the indicator is cell or tissue damage. In one embodiment the indicator is a symptom or sign of an autoimmune disease.

“Polynucleotide” is used herein interchangeably with “nucleic acid” to indicate a polymer of nucleosides. Typically, a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. The term “polynucleotide sequence” or “nucleic acid sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

“Polypeptide” refers to a polymer of amino acids. A protein is a molecule composed of one or more polypeptides. A peptide is a relatively short polypeptide, typically between about 2 and 60 amino acids in length. The terms “protein”, “polypeptide”, and “peptide” may be used interchangeably. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith may still be referred to as a “polypeptide”. Polypeptides of use in this invention may be purified from natural sources, produced in vitro or in vivo in suitable expression systems using recombinant DNA technology, synthesized through chemical means such as conventional solid phase peptide synthesis and/or using methods involving chemical ligation of synthesized peptides. The term “polypeptide sequence” or “amino acid sequence” as used herein can refer to the polypeptide material itself and/or to the sequence information (i.e. the succession of letters or three letter codes used as abbreviations for amino acid names) that biochemically characterizes a polypeptide. A polypeptide sequence presented herein is presented in an N-terminal to C-terminal direction unless otherwise indicated.

“Polypeptide domain” refers to a sequence of amino acids within a longer polypeptide. A polypeptide domain may exhibit one or more discrete binding or functional properties. Such properties include binding to one or more polypeptides, modulation of the binding or activity of one or more polypeptides, directing insertion of a polypeptide into a cellular membrane, recognition by an antibody or antigen binding fragment thereof, binding to a coenzyme, ion, or other ligand, catalytic activity or inhibition of catalytic activity, fluorescence and luminescence.

“Polypeptide variant” refers to any polypeptide differing from a naturally occurring polypeptide by amino acid insertion(s), deletion(s), and/or substitution(s), created using, e.g., recombinant DNA techniques. In some embodiments amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Insertions or deletions may range in size from about 1 to 20 amino acids, e.g., 1 to 10 amino acids. In some instances larger domains may be removed without substantially affecting function. In certain embodiments, the sequence of a variant can be obtained by making no more than a total of 5, 10, 15, or 20 amino acid additions, deletions, or substitutions to the sequence of a naturally occurring enzyme. In some embodiments, not more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

“Purified” or “substantially purified” as used herein denote that the indicated nucleic acid or polypeptide is present in the substantial absence of other biological macromolecules, e.g., polynucleotides, proteins, and the like. In one embodiment, the polynucleotide or polypeptide is purified such that it constitutes at least 90% by weight, e.g., at least 95% by weight, e.g., at least 99% by weight, of the polynucleotide(s) or polypeptide(s) present (but water, buffers, ions, and other small molecules, especially molecules having a molecular weight of less than 1000 daltons, can be present).

“Isolated” or “partially purified” as used herein refers to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

“Luminescent” as used herein refers to molecules that are intrinsically luminescent and those that generate a luminescent signal by acting on a substrate.

“Monitoring the interaction” refers to the process whereby the physical association of two or more polypeptides or a polypeptide and another entity are detected and, optionally, measured.

“Particle” as used herein encompasses spherical and nonspherical (e.g., rod-shaped, pyramid-shaped, ellipsoidal, irregular shaped) small fragments of material.

“Nanoparticle” indicates a microscopic particle with a longest dimension (e.g., diameter) of 1000 nm or less or less, e.g., 10-50 nm, 50-100 nm, 100-200 nm, 500-1000 nm.

“Recombinant polynucleotide” or “nucleic acid construct” refer to a polynucleotide that contains at least two nucleic acid sequences that are not found joined to one another in nature. The terms encompass entirely artificial sequences as well as sequences that contain at least one portion identical to or derived from a naturally occurring sequence.

“Recombinant polypeptide” refers to a polypeptide expressed from a recombinant polynucleotide. In some embodiments, a recombinant polypeptide contains at least two polypeptide segments joined together that are not found joined together in nature and thus has an overall amino acid sequence that does not exist in nature. A recombinant polypeptide can consist entirely of two or more polypeptide portions whose sequences are found in nature, (e.g., different naturally occurring polypeptides or portions thereof), can comprise or consist of one or more portions whose sequence is not found in any naturally occurring polypeptide, etc.

“RNAi” refers to a phenomenon whereby double-stranded RNA (dsRNA) trigger the specific degradation of a homologous mRNA having sufficient complementarity to one strand of the dsRNA such that the strand can guide cleavage of the mRNA in a protein complex called the RNA-induced silencing complex (RISC). In mammalian cells, the double-stranded portion of the RNA should be less than about 30 nucleotides in length to avoid inducing the interferon response. RNAi may be achieved by introducing an appropriate nucleic acid into cells or expressing the nucleic acid therein. Exemplary nucleic acids capable of directly or indirectly mediating RNAi are a short hairpin RNA (shRNA), short interfering RNA (siRNA), or microRNA precursor.

“Specific binding” means the specific recognition of one of two different molecules by or of the other compared to substantially less recognition of other molecules. In many instances, two or more molecular species have a particular affinity for each other that gives rise to a biological activity. Generally, the molecules have areas on their surfaces or in cavities giving rise to specific recognition between the two molecules. Exemplary of specific binding are antibody-antigen interactions, enzyme-substrate interactions, interactions between complementary polynucleotide, and so forth. In certain embodiments, binding of two molecules is considered specific if the equilibrium dissociation constant (Kd) of the molecules is 10⁻³ M or less, preferably 10⁻⁴M or less, more preferably 10⁻⁵ M or less, e.g., 10⁻⁶M or less, 10⁻⁷M or less, 10⁻⁸ M or less, or 10⁻⁹M or less under the conditions tested, e.g., under physiological conditions. It will be appreciated that the term “specific binding” may also be used to describe interactions between more than two molecules, between a complex comprising two or more molecules and another entity, etc.

“Subject”, as used herein, refers to an individual to whom an agent is to be delivered or from whom a sample is to be obtained, e.g., for experimental, diagnostic, and/or therapeutic purposes. Preferred subjects are mammals, e.g., rodents such as mice and rats, domesticated animals such as dogs and cats, non-human primates, or humans.

“Suitable conditions” refer to conditions appropriate for an event or process of interest to occur. For example, suitable conditions for synthesis of a recombinant polypeptide by a cell refer to the maintenance of cells comprising a polynucleotide encoding a recombinant polypeptide in growth medium and under environmental conditions (e.g., temperature, pH, redox and osmotic conditions, O₂ and CO₂ concentrations and presence or absence of an effective concentration of an appropriate expression-modulating agent) conducive to the synthesis of the recombinant polypeptide. Suitable conditions for performing an assay provided herein refer to conditions under which a complex comprising UNC93B and an UNC93B-dependent TLR would form and remain stable for sufficiently long and in sufficient amount, for example, to allow its detection and such conditions as are consistent with detection steps involved in the assay. Exemplary conditions under which a complex comprising UNC93B and an UNC93B-dependent TLR forms and remains stable are described in the Examples, and one of skill in the art would readily identify others. In certain embodiments, the conditions are those existing within living mammalian cells or lysates made therefrom. In some embodiments, physiological conditions, by which is meant conditions of pH, osmolarity, salt concentration, etc., similar to those existing in living cells or tissues, are used.

“TLR signaling” refers to any one of a series of biochemical events that ensues following binding of a stimulatory ligand to a ligand-binding domain of a TLR, including, for example, interaction of the TLR with any of a variety of adaptor proteins, triggering activation of downstream targets such as protein kinases and different transcription factors, leading to synthesis of a variety of proteins and, if in vivo, typically leading to a detectable biological effect in an animal.

An “UNC93B-dependent TLR” as used herein refers to a member of the Toll-like receptor family of polypeptides that under normal circumstances requires the presence of UNC93B for effective signaling, i.e., requires presence of UNC93B to properly respond to its ligand(s) and trigger the cascade of biochemical events leading to activation of NF-κB and cytokine production. TLRs 3, 7, 8, 9, and 13 are considered UNC93B-dependent TLRs for purposes of the present disclosure. In certain embodiments of the invention the UNC93B-dependent TLR is an intracellular TLR. By “intracellular TLR” is meant that the ectodomain of the TLR is located exclusively or primarily on or within organelles inside the cell rather than on the cytoplasmic membrane. In certain embodiments, the UNC93B-dependent TLR is an endosomal TLR, by which is meant that the TLR contains a domain that resides within the endosomal compartment and, optionally one or more domains that spans or all part of the endosomal membrane.

“Transcriptional activator” refers to a polypeptide that possesses the ability to activate transcription in a host cell, e.g. by facilitating recruitment and/or activation of the RNA polymerase II machinery of the cell. The transcriptional activator may bind to a transcription control sequence operably linked to an open reading frame. An exemplary transcriptional polypeptide is the herpes simplex virus VP16 protein, e.g., the acidic B42 domain.

“Selectable marker” refers to a gene, RNA, or protein whose presence within a cell confers a significant growth or survival advantage or disadvantage on the cell under certain defined culture conditions (selective conditions) such that maintaining the cell under such conditions allows the identification (and optionally the isolation) or elimination of cells that express the marker. Antibiotic resistance markers are a non-limiting example of a class of selectable marker. Examples include genes encoding enzymes that provide resistance to neomycin, zeocin, hygromycin, puromycin, etc. A second non-limiting class of selectable markers is nutritional markers. Such markers are generally enzymes that function in a biosynthetic pathway to produce a compound that is needed for cell growth or survival. Examples useful in mammalian cells include hypoxanthine phosphoribosyl transferase (HPRT), thymidine kinase (TK), and dihydrofolate reductase (DHFR). Examples in yeast include enzymes that participate in biosynthetic pathways for synthesis of amino acids such as leucine, histidine, etc.

“Treating”, as used herein, refers to providing any medical or surgical management of a subject in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a disease or clinical condition, e.g., in order to reverse, alleviate, reduce the severity of, and/or inhibit or prevent its progression and/or to reverse, alleviate, reduce the severity of, and/or inhibit or one or more symptoms or manifestations of the condition. An agent can be administered to a subject who has developed a disease or conditions or is at increased risk of developing such a disease or condition relative to a member of the general population. A composition of this invention can be administered prophylactically, i.e., before development of any symptom or manifestation of the condition. Typically in this case the subject will be at increased risk of developing the condition. “At increased risk” of means that (i) the subject has at least one art-recognized risk factor for developing the condition relative to a member of the general population (e.g., family history, exposure to an infectious agent, etc.) and/or (ii) has exhibited a symptom, sign (either clinical or based on a laboratory or other diagnostic test) indicative of an increased likelihood of developing the condition relative to an otherwise similar individual not exhibiting such sign or symptom and/or (iii) would benefit from prophylactic therapy in the sound judgement of a health care practitioner.

“Vector” is used herein to refer to a nucleic acid or a virus or portion thereof (e.g., a viral capsid) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral capsids. Plasmid vectors typically include an origin of replication and one or more selectable markers. Plasmids may include part or all of a viral genome (e.g., a viral promoter, enhancer, processing or packaging signals, etc.). Viruses or portions thereof (e.g., viral capsids) that can be used to introduce nucleic acid molecules into cells are referred to as viral vectors. Useful viral vectors include adenoviruses, adeno-associated virus, retroviruses, lentiviruses, vaccinia virus, herpes simplex virus, and others. Viral vectors may or may not contain sufficient viral genetic information for production of infectious virus when introduced into host cells, i.e., viral vectors may be replication-defective, The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are sometimes referred to as viral vectors. Expression vectors include regulatory sequence(s), e.g., expression control sequences such as a promoter, sufficient to direct transcription of an operably linked nucleic acid. An expression vector comprises sufficient cis-acting elements for expression; other elements for expression can be supplied by the host cell or in vitro expression system. Such vectors typically include one or more appropriately positioned sites for restriction enzymes, to facilitate introduction of the nucleic acid to be expressed into the vector.

### II. Overview

As described herein, TLRs 3, 7, 9 and 13 physically interact with the ER-resident membrane protein UNC93B, and the interaction is essential for normal signaling of such TLRs. Introducing the point mutation H412R into UNC93B abolishes the interactions between UNC93B and these TLRs without affecting the expression or stability of the TLRs (see Examples).

The present disclosure establishes that TLR7 and TLR9 physically interact with UNC93B in a variety of immune system cells including splenocytes and bone marrow-derived dendritic cells. Further, by expressing chimeric TLRs, TLR3 and TLR9 are shown to bind to UNC93B via their transmembrane domains. These results reveal that a physical association between UNC93B and TLRs in the ER is essential for proper TLR signaling. Therefore, the failure of the mutant UNC93B to interact with TLRs can account for the compromised immune phenotype of the mice and humans having the mutation.

The invention encompasses the recognition that modulating the interaction between one or more UNC93B-dependent TLR(s) and UNC93B is an effective means of modulating TLR signaling via such TLR(s). The UNC93B-TLR interaction thus represents a novel target for the identification of immunomodulators, and agents that alter this interaction represent a new class of immunomodulating agents. Methods of modulating an activity of an immune system cell are provided. In some embodiments, the methods comprise contacting the cell with an agent that modulates interaction between UNC93B and an UNC93B-dependent TLR. Methods of modulating an activity of the immune system of a subject are also provided. In some embodiments, the methods comprise administering to the subject an agent that modulates interaction between UNC93B and an UNC93B-dependent TLR. A variety of different methods are provided for identifying such agents and also provides reagents useful for performing the methods. Also provided are methods of using the agents. Since UNC93B physically interacts with multiple UNC93B-dependent TLRs, compounds that alter the interaction of UNC93B with more than one member of this class, e.g., with 2, 3, or 4 TLRs can be identified. Thus, signaling of multiple TLRs may be inhibited with a single compound. Administering such a compound to an individual would provide a means to modulate the individual's immune response to a variety of different agents with a single compound rather than needing to target individual UNC93B-dependent TLRs. TLRs that do not physically interact with UNC93B and/or do not require UNC93B for their activity would be unaffected. For example, a compound that inhibits interaction between UNC93B and TLRs 3, 7, 8, and 9 would preserve the ability of the individual to mount an immune response to microbial components that are recognized by TLR4.

### III. Assay Compositions, Systems, and Methods for Identifying an Immunomodulator

Methods (also referred to as “assays”, “screens”, etc.) comprising a sequence of steps designed and adapted to identify a candidate agent that modulates the signaling activity of TLR3, 7, 8, 9, or 13 by modulating the physical interaction of such TLR(s) with UNC93B are provided. Further provided are assay compositions suitable for performing the assays. In some embodiments, the assays are designed to identify a candidate agent that modulates the signaling activity of at least two of these TLRs. In some embodiments, the assays are designed to identify a candidate agent that modulates the signaling activity of TLR3, 7, 8, and 9. In some embodiments, the assays are designed to identify a candidate agent that modulates the physical interaction of UNC93B with TLR3, 7, 8, or 9. In some embodiments the assays are designed to identify a candidate agent that modulates the physical interaction of UNC93B with at least 2, or at least 3 of these TLRs. In some embodiments, the assays are designed to identify a candidate agent that modulates the physical interaction of UNC93B with TLR3, 7, and 9.

Agents that decrease the physical interaction of UNC93B with TLR3, 7, and/or 9 are of particular interest. An agent that significantly inhibits the physical interaction between UNC93B and a particular TLR will inhibit signaling by that TLR. Certain agents that strengthen the physical interaction between UNC93B and a particular TLR enhance signaling by that TLR while other agents inhibit signaling by that TLR. One of skill in the art will be able to determine whether an agent inhibits or enhances signaling by a TLR using methods known in the art, such as those described herein for assessing TLR signaling. Efforts to identify agonists and antagonists of receptors have traditionally focused on the ligand-binding domain. Such efforts have sought, for example, to identify ligands that would prevent binding of a naturally occurring ligand without activating the receptor or that would mimic the activity of a naturally occurring ligand. The invention identifies the transmembrane domain of TLRs 3, 7, 8, and 9, as an important target for the identification of agents that modulate TLR signaling. In particular, agents that bind to the transmembrane domain of TLR3, 7, 8, and/or 9 may prevent binding of the TLR to UNC93B and thereby inhibit signaling via the TLR. The invention encompasses any screening assay useful to identify an agent that binds to a transmembrane domain of TLR3, 7, 8, and/or 9.

Assays compositions, systems, and components thereof (e.g., nucleic acid constructs, vectors, cell lines) useful for performing the inventive screening methods are provided. Kits comprising one or more components useful for performing an inventive screen are provided. The components include a variety of genetically modified cells and cell lines. It should be understood that where this application refers to or describes a genetically engineered cell, the invention also provides a “cell line” whose members have the recited characteristics of the cell. It should be understood that where this application describes an assay, assay composition(s) and assay system(s), of use for performing the assay and each component of the assay composition, all combinations thereof, as well as methods and reagents for preparing the composition and its components are provided.

The sequences of UNC93B-dependent TLR3, 7, 8, 9, and 13 polypeptides and the sequence of UNC93B polypeptide are known in the art. Table 1 shows the Gene ID of genes encoding these polypeptides in mouse and human along with accession numbers for the encoded polypeptides. It is noted that UNC93B is also referred to in the art as UNC93B1. FIG. 9 shows exemplary human and mouse sequences. For purposes of the present invention, these sequences will be considered “wild type”. In some embodiments, “UNC93B” and “UNC93B-dependent TLR” refer to human UNC93B and human UNC93B-dependent TLRs. One of skill in the art will readily be able to identify UNC93B and TLR sequences from other species using publicly available databases such as GenBank. It will be understood that DNA sequence polymorphisms that lead to changes in the amino acid sequences of the proteins mentioned herein will exist among different members of a population and that mutations may arise in individual cells. One skilled in the art will appreciate that these variations in one or more nucleotides (up to about 3-5% of the nucleotides) of the nucleic acids encoding a particular protein may exist among individuals of a given species due to natural allelic variation. All such naturally occurring nucleotide variations and polypeptides having the resulting amino acid polymorphisms are within the scope of this invention. It will be understood that most such variations will have little or no effect on the physiologically important activity or activities of interest of the polypeptide and may thus be considered “wild type” as well. In the case of a TLR polypeptide, an activity of interest is mediating an immune system response to a PAMP, e.g., initiating a signaling pathway leading to activation of NF-κB and production of proinflammatory cytokines. In the case of an UNC93B polypeptide, activities of interest include binding to an UNC93B-dependent TLR and facilitating signaling via such TLR. It will be understood that nucleic acids encoding UNC93B or an UNC93B-dependent TLR could be obtained by screening (e.g., under art-recognized high stringency conditions) or performing PCR on genomic or cDNA libraries using, e.g., probes comprising at least a portion of an UNC93B or UNC93B-dependent TLR polynucleotide sequence. It will further be appreciated that as a result of the degeneracy of the genetic code, many variations of the specific naturally occurring nucleic acid sequences that encode UNC93B or an UNC93B-dependent TLR are of use in the invention for purposes of expressing an UNC93B or UNC93B-dependent TLR polypeptide. In some embodiments, the sequence is deliberately changed in order to accommodate codon usage preference in a particular host cell.

TABLE 1

| | Gene ID/mRNA/Protein | |
|---|---|---|
| | Acc. No. | Gene ID/Protein Acc. No. |
| Gene | (Homo sapiens) | (Mus musculus) |
| UNC93B | 81622/NM_030930/ | 54445/NM_019449/NP_062322 |
| | NP_112192 | |
| TLR3 | 7098/NM_003265/ | 142980/NM_126166/NP_569054 |
| | NP_003256 | |
| TLR7 | 51284/NM_016562/ | 170743/NM_133211/NP_573474 |
| | NP_057646 | |
| TLR8 | 51311/NM_016610/ | 170744/NM_133212NP_573475 |
| | NP_057694 | |
| | (isoform 1) | |
| | NM_138636/ | |
| | NP_619542.1 | |
| | (isoform 2) | |
| TLR9 | 54106/NM_017442/ | 81897/NM_031178/NP_112455 |
| | NP_059138 | |
| TLR13 | N/A | 279572/NM_205820/NP_991389 |

In some embodiments, the UNC93B-dependent TLR(s) and UNC93B polypeptides are wild type polypeptides, e.g., having the sequences listed under the accession numbers of Table 1 and/or presented in FIG. 9. It will also be appreciated that a variety of functional variants of full length wild type UNC93B polypeptide and TLR polypeptides could be used in the inventive assay systems and methods. In addition to naturally-occurring allelic variants of the TLR and UNC93B sequences that may exist in the population, it will be appreciated that, as is the case for virtually all proteins, a variety of changes can be introduced into the nucleotide sequences listed under the accession numbers in Table 1, thereby leading to changes in the amino acid sequence of the encoded polypeptides, without substantially altering the functional (biological) activity of the polypeptides. Such variants are included within the scope of the terms “UNC93B polypeptide” and “UNC93B-dependent TLR polypeptide”. One example of variants are isoforms arising from the gene encoding the sequences listed in Table 1, e.g., as a result of alternate splicing.

The variant could be, e.g., a polypeptide at least 80%, 85%, 90%, 95%, 98%, or 99% identical to full length wild type UNC93B. The variant could be a fragment of fully length wild type UNC93B. The variant could be a polypeptide at least 80%, 85%, 90%, 95%, 98%, or 99% identical to a fragment of wild type UNC93B, wherein the fragment is at least 50%, 60%, 70%, 80%, 85%, 90%, 95%, 98%, or 99% as long as the full length polypeptide or a domain thereof having an activity of interest. In some embodiments the domain is at least 100, 200, 300, 400, or 500 amino acids in length. The variation allowed may be experimentally determined by making insertions, deletions, or substitutions in a polynucleotide encoding UNC93B or a TLR, e.g., using recombinant DNA techniques and assaying the resulting recombinant polypeptide variants for an activity of interest. For example, as described herein, the H412R mutation eliminated the physical interaction between UNC93B and TLRs 3, 7, and 9. Mutations in genes encoding various TLRs are known in the art to eliminate or substantially reduce the activity of the protein. In some embodiments such mutations are avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature polypeptide, by which is meant a polypeptide that has had one or more portions such as a signal peptide removed during normal intracellular proteolytic processing (e.g., co-translational or post-translational processing). In some embodiments the TLR polypeptide lacks one or more leucine-rich repeats. In some embodiments the UNC93B or TLR polypeptide is a chimeric polypeptide, by which is meant that it contains domains from two or more different species. For example, the polypeptide could contain a human ectodomain with other portions of the polypeptide having murine sequence, or the polypeptide could contain one or more human transmembrane domain with other portions of the polypeptide having murine sequence.

A variant of a wild type UNC93B polypeptide of use in the assays of the present invention is capable of binding to at least one UNC93B-dependent TLR. Similarly, a variant of a wild type UNC93B polypeptide of use in the present invention is capable of binding to at least one UNC93B-dependent TLR and, in some embodiments, to at least 2, 3, or 4 UNC93B-dependent TLRs. One of skill in the art will be aware of, or will readily be able to ascertain, whether a particular variant is a functional variant using, e.g., the assays described herein or others known in the art. For example, the ability of a variant of a wild type UNC93B polypeptide to bind to a TLR polypeptide can be assessed as described in the Examples. The ability of a variant of a wild type TLR to trigger activation of NF-κB and/or to trigger cytokine production in response to a ligand can be determined as known in the art and described herein. In certain embodiments of the invention a functional variant has at least 50%, 60%, 70%, 80%, 90%, 95% or more of the activity of the full length wild type polypeptide. Variants having lesser degrees of activity, or no detectable activity, are of use as negative controls.

A variety of different assays to identify agents that modulate the interaction between UNC93B and an UNC93B-dependent TLR are provided. A wide variety of assays may be used, including in vitro protein-protein binding assays using labeled proteins, electrophoretic mobility shift assays, gel filtration chromatography, immunoassays (e.g., ELISA, co-immunoprecipitation, Western blot, protein microarray) for protein binding. Purified UNC93B and TLR polypeptides may also be used for determination of three-dimensional crystal structure, which can be used for modeling intermolecular interactions and design of candidate agents.

In some embodiments, living cells are contacted with a candidate agent. A readout is obtained from the cells after maintaining them in the presence of the candidate agent under conditions suitable for physical interaction of UNC93B and TLRs to occur. Measurements can be made on individual cells or on groups of cells, e.g., cells within a given field of view, well of a microwell plate, etc. Methods employing intact cells may make use of microscopy systems (e.g., fluorescence microscopy) or flow cytometry (e.g., fluorescence activated cell sorting). In some embodiments automated digital microscopy system is used to monitor cells after contacting them with a test agent. In some embodiments, cells are lysed after being contacted with the agent, and UNC93B and/or TLR polypeptides or UNC93B/TLR complexes are detected, optionally after being isolated from the lysate. In some embodiments, the candidate agent is added to a cell lysate or to an assay composition containing partially or substantially purified UNC93B and TLR polypeptides, and the effect of the agent on binding of UNC93B and one or more TLR polypeptides is assessed.

The parameters detected in a screening assay may be compared to a suitable reference. A suitable reference may be an assay run previously, in parallel or later that omits the candidate agent. A suitable reference may also be an average of previous measurements in the absence of the candidate agent or in the presence of a different concentration of the candidate agent. In general, the components of a screening assay mixture may be added in any order consistent with the overall activity to be assessed, but certain variations may be preferred.

The invention provides “interaction-specific” assays of use to identify agents that modulate physical interaction between UNC93B and the TLR. In certain embodiments these assays provide a readout that directly assesses the extent of the interaction between UNC93B and an UNC93B-dependent TLR. In some embodiments, the assays measure the interaction between UNC93B and the TLR in the presence or absence of a candidate agent or in the presence of differing concentrations of the candidate agent. The extent of interaction is compared, and agents that cause an increase or decrease in the extent of the interaction are identified. Agents identified by an interaction-specific assay could modulate the interaction between UNC93B and the TLR by a variety of mechanisms. Some agents directly contact, e.g., bind to, UNC93B and inhibit binding of the TLR. Other agents directly contact, e.g., bind to, a TLR and inhibit binding of UNC93B. Some agents bind to and stabilize an UNC93B/TLR complex. Some agents do not directly contact UNC93B or a TLR. Instead, such agents may modulate the activity or expression of a third polypeptide whose activity is needed for the physical interaction of UNC93B and a TLR. The third polypeptide may, for example, participate in proper folding, localization, or post-translational modification of UNC93B or the TLR or may be an as yet unidentified third component of an UNC93B/TLR complex. Alternately or additionally, agents identified using certain interaction-specific assays may affect the synthesis, localization, folding, or processing of UNC93B and/or the TLR. In some embodiments, agents that reduce or increase synthesis, stability, and/or steady state levels of UNC93B or an UNC93B-dependent TLR are excluded, e.g, by controlling or normalizing for total levels of these polypeptides, by assessing the effect of the agent on level of these proteins, etc. In some embodiments agents that alter the localization of at least a portion of the UNC93B or an UNC93B-dependent TLR made by the cell are excluded. In other embodiments such agents are of interest. Any interaction-specific assay known in the art could be adapted to detect an agent that modulates interaction between an UNC93B-dependent TLR and UNC93B. Such assays are an aspect of the invention. In some embodiments the assay is a “two-hybrid” or “three-hybrid” assay. In some embodiments the assay is adapted for use with polypeptides that are attached to or span a cell membrane.

Some methods of the invention include an “interaction-independent” assay, which is used to identify agents that modulate TLR signalling without regard to the mechanism by which TLR signalling is modulated, followed by an interaction-specific assay to determine whether an agent identified using the interaction-independent assay does indeed modulate the interaction between UNC93B and an UNC93B-dependent TLR. An agent identified using an interaction-independent assay modulates TLR signaling by a mechanism that may or may not involve modulating the physical interaction between UNC93B and the TLR. For example, such agents could inhibit a kinase whose activity is required for TLR signalling or could bind to the ligand-binding domain of a TLR and block its interaction with a PAMP. A number of interaction-independent assays are known in the art and could be used in the practice of the present invention. Exemplary interaction-independent and interaction-specific assays are discussed below. Exemplary interaction-specific and interaction-independent assays are described below.

A. Interaction-Independent Assays

Interaction-independent assays include any assay that can be used to assess the ability of a candidate agent to modulate TLR signalling. One class of assays assesses the ability of a candidate agent to modulate a TLR-mediated functional response of a cell to a stimulating agent such as a PAMP. A suitable functional response is production of any one or more proteins whose production is stimulated by the TLR in response to recognition of a ligand. Such proteins are referred to herein as “TLR-induced proteins”. One of skill in the art will be aware of such proteins, which include a variety of cytokines, cytokine receptors, and other proteins (e.g., cell adhesion molecules such as selectins), and methods for their measurement.

A variety of methods could be used to measure production of a TLR-induced protein. In one embodiment, an ELISA assay is used. Such assays are well known in the art, and kits for performing the assays are commercially available. Other methods that involve capture of the TLR-induced protein by a binding reagent (e.g., an antibody) attached to a solid phase (e.g., particle, filter, slide), followed by detection of the bound protein using a second binding reagent (optionally labeled with a detectable moiety or enzyme capable of generating a colorimetric, fluorescent, or other detectable signal) are also of use. In an exemplary embodiment a monoclonal antibody specific for a cytokine is covalently linked to a bead, which captures the TLR-induced protein. A complementary biotinylated monoclonal cytokine antibody then completes the immunological sandwich and the reaction is detected with streptavidin-phycoerythrin. In some embodiments, the assay is multiplexed and is capable of detecting multiple cytokines. For example, a plurality of fluorescent beads, each comprising a different fluorescent dye and having a capture reagent for a different cytokine attached thereto, could be used. Individual cytokines could be measured by separating the beads based on their differential fluorescence. In some embodiments production of mRNA encoding a TLR-induced protein is assessed. Any suitable mRNA detection method can be used. Many such methods are based on hybridization of the mRNA to a complementary nucleic acid, which may be detectably labeled, or on amplification of the mRNA following hybridization of specific primers thereto. Functional assays for cytokine production could also be used. For example, certain cytokines stimulate or inhibit proliferation of various cell types.

Other suitable interaction-independent assays include cell-base transcriptional reporter assays. Such assays are widely used to monitor the cellular events associated with signal transduction and gene expression. Reporter assays couple the biological activity of a target to the expression of a readily detected enzyme or protein reporter. Based upon the fusion of transcriptional control elements to any of a variety of reporter genes, such systems “report” the effects of a cascade of signaling events on gene expression inside cells. One or more response elements for a particular transcription factor or complex of interest can be inserted upstream of the reporter gene to couple its expression to activation of a specific pathway in living cells, e.g., in response to extracellular or intracellular signaling molecules or biochemical events. Typical response elements include a binding site for a transcription factor that is a downstream component of a signal transduction pathway of interest. Many transcriptional reporter genes and their application for a variety of purposes including drug screening are known in the art. Examples include systems based on GFP and other fluorescent proteins (e.g., those produced by Aequoria, Discosoma, Anemonia, or other species), luciferase (e.g., firefly or Renilla luciferase), and numerous more recently developed bioluminescent or fluorescent reporters. Systems based on enzyme reporters such as beta-galactosidase, alkaline phosphatase, chloramphenicol acetyltransferase, etc., are also of use. In one embodiment the enzyme is secreted.

TLR signaling activates the transcription factor NF-κB, which in turn activates transcription from promoters that comprise one or more NF-κB response elements. To perform an assay for an agent that modulates TLR signaling, an NF-κB reporter construct is introduced into cells. The cells are contacted with a candidate agent and typically also with a ligand for the TLR or other stimulus that would activate the TLR signaling pathway. Known ligands or stimuli for TLR3 include polyinosine-polycytidylic acid (poly(I:C)), a synthetic analog of double-stranded RNA (dsRNA); for TLR7 include certain guanosine and adenine derivatives and various imidazoquinolines (specific examples are CL087, S-28463, gardiquimod, imiquimod, resiquimod (R-848) and loxoribine); for TLR8 include some of the afore-mentioned TLR7 agonists; for TLR9 include CpG oligodeoxynucleotides (ODN). The effect of the candidate agent on the signal generated by the reporter is assessed. Agents that cause an increase or decrease in the signal are identified as modulators of the TLR signaling pathway. Agents having nonspecific effects on cell viability, transfection efficiency, or overall protein expression can be eliminated from consideration using appropriate controls. Additional assays can be performed to determine whether the agent affects the expression, localization, or activity of specific protein(s) involved in the signaling pathway.

Following identification of an agent that modulates TLR signaling, any of a number of different assays can be performed in order to confirm that an agent specifically affects the physical interaction between UNC93B and an UNC93B-dependent TLR. Suitable assays are described in the following section. In some embodiments, one or more interaction-specific assays will be performed to identify agents that modulate the physical interaction between UNC93B polypeptide and any 1, 2, 3, or more UNC93B-dependent TLRs.

B. Interaction-Specific Assays

Assays that can be used to specifically identify or confirm agents that modulate the physical interaction between UNC93B and an UNC93B-dependent TLR include co-immunoprecipitation or “pull-down” assays. In certain embodiments such methods include immobilizing an UNC93B polypeptide or an UNC93B-dependent TLR polypeptide on a solid support and subsequently contacting a solution containing an UNC93B-dependent TLR polypeptide or an UNC93B polypeptide, respectively, with the solid support. If a physical interaction occurs, the UNC93B-dependent TLR or an UNC93B polypeptide, respectively, will be retained in association with the solid support. It can then be detected using any suitable method, e.g., by use of an antibody that specifically binds to it. In other embodiments an antibody or other reagent that specifically binds to UNC93B-dependent TLR or an UNC93B polypeptide is used to isolate, e.g., immunoprecipitate, the UNC93B-dependent TLR or an UNC93B polypeptide from a cell lysate. In the case of a binding reagent that specifically binds to the UNC93B-dependent TLR or an UNC93B polypeptide, presence of the other polypeptide in the precipitated material is assessed, e.g., using appropriate techniques such as mass spectrometry, immunological techniques, etc. Co-immunoprecipitation or pull-down assays may be used to identify an agent that modulates the interaction between UNC93B and an UNC93B-dependent TLR or to confirm that an agent identified as a modulator of TLR signaling (e.g., in an interaction-independent assay) acts by modulating the interaction between UNC93B and an UNC93B-dependent TLR. The contacting or immunoprecipitation is performed in the presence of a candidate agent. If the polypeptide that is attached to the solid support or immunoprecipitated is the UNC93B polypeptide, then the amount of the UNC93B-dependent TLR pulled down or co-precipitated is assessed, and vice versa. Thus agents that specifically increase or decrease the strength of the physical interaction are identified. It will be appreciated that either or both of the polypeptides may further comprise a moiety such as an epitope tag or detectable polypeptide.

A variety of proximity-dependent assays that specifically detect a physical interaction between UNC93B and a TLR are provided. The proximity-dependent assays may be particularly well suited for screening a plurality of compounds (e.g., a compound library) to identify those that modulate TLR signalling by modulating the physical interaction between UNC93B and an UNC93B-dependent TLR. The assays utilize a proximity-dependent reporter system comprising two moieties which, when brought into close proximity with one another, result either directly or indirectly in a detectable signal. A first moiety is physically associated with an UNC93B polypeptide. A second moiety is physically associated with an UNC93B-dependent TLR polypeptide. When the UNC93B and TLR polypeptides physically interact, the first and second moieties are brought into close proximity with one another, resulting either directly or indirectly in a detectable signal. “Indirectly” in this context means that the moeities require the presence of an additional assay component, e.g., a substrate, in order for a detectable signal to result.

In certain embodiments, the detectable signal is emission or absorption of electromagnetic energy. In certain embodiments the electromagnetic energy is light having a wavelength that in the range from infrared to ultraviolet (e.g, about 1 mm-1 nm). In certain embodiments the energy is light in the infrared range (e.g., about 1 mm-about 400 nm). In certain embodiments the energy is light in the visible range (e.g., about 400-about 700 nm). In certain embodiments the energy is light in the near UV range (about 400-about 200 nm) or in the far or vacuum UV range (about 200-about 10 nm). A variety of different proximity-dependent reporter systems can be used. The following sections describe a number of these reporter systems and assays in which they are used to detect an agent that modulates the interaction between UNC93B and a TLR.

(1) Reporter Systems and Assays Based on Resonance Energy Transfer (RET)

In certain embodiments, the proximity-dependent reporter system uses the phenomenon of resonance energy transfer to produce a signal in response to physical interaction of UNC93B and a TLR. Various embodiments of the invention employ fluorescence resonance energy transfer (FRET), luminescence resonance energy transfer (LRET), or bioluminescence resonance energy transfer (BRET). FRET is a distance-dependent interaction between the electronic excited states of two molecules in which excitation is transferred from a donor moiety to an acceptor moiety without emission of a photon, resulting in emission from the FRET acceptor. In order for FRET to occur the donor and acceptor should be in very close proximity, e.g., less than approximately 10 nm, and the absorption spectrum of the acceptor must overlap the fluorescence emission spectrum of the donor. LRET has similarities to FRET but uses a luminescent moiety, e.g., a lanthanide as the energy-transfer donor. BRET is analogous to FRET but uses a luminescent or luminescence-generating biomolecule such as luciferase, aequorin, or a derivative thereof as an energy donor and a fluorescent moiety, e.g., a biomolecule such as green fluorescent protein (GFP) as the acceptor, thus eliminating the need for an excitation light source (reviewed in Pfleger, K. an Eidne, K., Nature Methods, 3(3), 165-174, 2006). In a typical BRET assay, oxidation by the donor of a suitable substrate results in transfer of energy to the acceptor, resulting in energy emission by the acceptor. BRET²™ is a form of BRET that is based on the transfer of resonant energy from a bioluminescent donor protein to a fluorescent acceptor protein using Renilla luciferase (Rluc) as the donor, a coelenterazine derivative referred to as DeepBlueC (Perkin-Elmer) as the substrate, and a mutant of the green fluorescent protein 2 (GFP2) as the acceptor molecule.

A proximity-dependent assay based on RET includes the following two components: (i) a first polypeptide comprising an UNC93B polypeptide having a RET donor or acceptor physically associated therewith; and (ii) a second polypeptide comprising a TLR polypeptide having a RET acceptor or donor physically associated therewith. It will be appreciated that if the UNC93B polypeptide has a RET donor associated therewith, then the TLR polypeptide should have a RET acceptor associated therewith, while if the UNC93B polypeptide has a RET acceptor associated therewith, then the TLR polypeptide should have a RET donor associated therewith. The assay is performed by maintaining the first and second polypeptides in an assay composition under conditions in which physical interaction of the UNC93B and TLR portions of the polypeptides could occur, assuming that no agent capable of preventing, inhibiting, or disrupting such association is present. In the absence of such an agent, the UNC93B and TLR portions of the two polypeptides associate, thereby bringing the RET donor and acceptor into close proximity to each other. The composition is exposed to electromagnetic radiation having a suitable wavelength to excite the RET donor moiety, which then emits energy at its characteristic emission wavelength. This energy is absorbed by the RET acceptor, resulting in a detectable signal (e.g., acceptor emission, donor quenching, etc.) The assay is performed by adding a candidate agent to the assay composition and determining whether presence of the agent has a detectable effect on the signal, e.g., whether the signal is enhanced or reduced in the presence of the agent versus its absence or presence at a greater or lesser concentration. If the signal is detectably different in the presence of the agent or is affected by varying the concentration of the agent, it may be concluded that the agent either directly or indirectly affects the physical interaction between UNC93B and the TLR. The assay composition could comprise living cells, cell lysates, or isolated and optionally purified polypeptides. The invention encompasses embodiments in which first and second isolated polypeptides and the candidate agent are added to an assay vessel in any order.

FRET measurements involve detecting acceptor emission, donor quenching (decreased emission from the FRET donor), and/or an alteration in the fluorescence lifetime of the donor. Assays of the invention can make use of increases in acceptor emission, decreases in acceptor emission, donor quenching, reduction in donor quenching, and/or increase or decrease in fluorescence lifetime of the donor to detect increased proximity or decreased proximity of an UNC93B and a TLR polypeptide from each other. Nonfluorescent acceptors, also referred to as quenchers are of use and include dabcyl and QSY dyes. Such molecules are capable of absorbing the energy of an excited fluorescent label when located in close proximity and of dissipating that energy without the emission of visible light.

A wide variety of different RET donors and acceptors are of use in the present invention. RET donors and acceptors include molecules in various classes including: organic materials (including “traditional” dye fluorophores, quenchers, and polymers; inorganic materials such as metal chelates, metal and semiconductor nanocrystals (e.g., “quantum dots”, and fluorophores of biological origin such as fluorescent proteins and amino acids; and biological compounds that exhibit bioluminescensce upon enzymatic catalsysis. Specific examples of RET donors and acceptors include acridine dyes; Alexa dyes; BODIPY, cyanine dyes; fluorescein and derivatives thereof; rhodamine derivatives thereof; GFP and derivatives thereof; blue, sapphire, yellow, red, orange, and cyan fluorescent proteins and derivatives thereof; monomeric red fluorescent protein (mRFP1) and derivatives such as those known as “mFruits”, e.g., mCherry, mStrawberry, etc., quantum dots, etc. Organic UV dyes are typically pyrene, naphthalene, and coumarin-based structures. Visible/near IR dyes include a number of fluorescein, rhodamine, and cyanine-based derivatives.

Certain amino acids, particularly those having an aromatic ring, and polypeptides that contain them, are intrinsically fluorescent. The strong UV absorbance of proteins at 280 nm and emission at 340-360 nm originates primarily from the indole ring of the tryptophan (Trp) residue with lesser contributions from tyrosine (Tyr) and phenylalanine (Phe). In some embodiments Trp, or a moiety containing multiple Trp, Tyr, and/or Phe residues, is used as a RET donor. These amino acids can easily be substituted or inserted into, or added to, an UNC93B or TLR polypeptide. Assays employing amino acids for purposes of RET may advantageously be performed using at least partially purified polypeptides rather than intact cells or cell lysates containing many other polypeptides that could result in a large background signal. In some embodiments of the invention the proximity-dependent reporter system does not employ a naturally fluorescent amino acid as the RET donor or acceptor.

One of skill in the art will readily be able to select appropriate RET donor and acceptor pairs. There are numerous resources in the literature to assist with such selection. See, e.g., The Handbook—A Guide to Fluorescent Probes and Labeling Technologies, 10^{th} edition (Invitrogen Corp.), which describes numerous fluorescent and otherwise detectable molecules and methods for their use and modification. See also Trinquet, E. and Mathis, G., Mol. Biosyst., 2: 380-387, 2006; Sapsford, K, et al., Angewandte Chemie Int. Ed., 45: 4562-4588, 2006 for further information on RET. A nonlimiting list of exemplary FRET donor/acceptor pairs includes: coumarin/fluorescein; fluorescein/rhodamine; Cy3.5/Cy5; Alexa fluors/GFP; YFP/GFP; CYPet/YPet, etc. Dye/quencher combinations include rhodamine/Dabcyl and Cy3/QSY9. RET donor and acceptor moieties are commercially available from a number of suppliers including Invitrogen Corp., Amersham Biosciences, Pierce, Biosearch Technologies, etc.

One of skill in the art will recognize that certain RET donors and acceptors will be suitable for cell-based assays; some will be suitable for assays employing isolated or purified polypeptides; and some will be suitable for both types of assays. For example, if the assay is to be conducted using whole cells, it may be advantageous to select RET donors and acceptors that are polypeptides (referred to herein as “RET polypeptides”), allowing them to be incorporated as part of a fusion protein. Such fusion proteins, which include a first portion comprising UNC93B polypeptide or a TLR polypeptide and a second portion comprising a RET polypeptide, are an aspect of the invention. A polypeptide containing an UNC93B or TLR polypeptide and a RET polypeptide can be encoded by a nucleic acid construct that comprises an open reading frame (ORF) encoding the RET polypeptide in frame with an ORF encoding the UNC93B or TLR polypeptide. The two ORFs may be separated by a polynucleotide sequence that encodes a linker region. The linker region is typically a short polypeptide chain (e.g., 1-50 amino acids, e.g., 5-25 or 5-15 amino acids). The precise length and sequence are typically not critical. Small amino acid residues such as serine, glycine, and alanine are of use. Examples include (Gly-Ser)n, (Thr-Ser-Pro)n, (Gly-Gly-Gly)n, (Gly-Ala)n, and (Glu-Lys)n, wherein n is 1 to 15, and variants in which any of the amino acid residues is repeated with the proviso that the total number of amino acids is within one of the afore-mentioned ranges. Nucleic acid constructs that encode such fusion proteins are an aspect of the invention. The resulting ORF may be translated in vitro or in cells to produce the fusion protein. In some embodiments the ORF encoding the RET polypeptide is appended at the 5′ or 3′ end of the ORF that encodes the UNC93B or UNC93B-dependent TLR polypeptide. It may be desirable to append the RET donor or acceptor at the terminus opposite the ligand binding domain of the TLR. The nucleic acid construct may be inserted into a vector in operable association with expression control elements such as a promoter, promoter/enhancer, etc. Appropriate polyadenylation and termination signals, etc., may be included. The vector is introduced into an appropriate host cell using art-accepted methods appropriate for the host cell. The ORF is transcribed and translated in the cell to produce the fusion protein. Such vectors and host cells containing them are an aspect of the invention.

Recombinant DNA techniques well known in the art are employed to produce a polypeptide comprising an UNC93B or UNC93B-dependent TLR polypeptide and a RET polypeptide. See, e.g., Current Protocols in Molecular Biology, Current Protocols in Immunology, Current Protocols in Protein Science, Sambrook, et al, supra. Briefly, in an exemplary method, nucleic acids encoding the UNC93B or TLR polypeptide are obtained from any suitable source (e.g., by cloning from a cDNA library, by PCR amplification using appropriate primers and a template such as mammalian cDNA or from a plasmid containing the coding sequence, etc.). The nucleic acids can also be chemically synthesized. Nucleic acids encoding the RET donor and acceptor are also obtained. These are widely available from commercial sources. The nucleic acids encoding the UNC93B or TLR polypeptide and the RET donor or acceptor polypeptide are inserted in frame with one another into a vector containing expression control signals suitable for directing expression in cells of the desired type, e.g., mammalian cells. They may be inserted as a single unit or as individual units in frame with one another. It will be appreciated that the details by which any particular nucleic acid construct is made can vary, and any suitable method can be employed. Expression vectors designed for convenient insertion of a polynucleotide of interest in frame with a sequence encoding the RET donor or acceptor are known in the art.

The resulting vector typically includes a transcriptional unit comprising (i) genetic element(s) having a regulatory role in gene expression, e.g., expression control sequences such as, promoters, operators, or enhancers, operatively linked to (ii) a “coding” sequence which is transcribed to produce an RNA that can be translated into a polypeptide comprising an UNC93B or TLR polypeptide and a RET polypeptide, and (iii) appropriate transcription initiation and termination sequences. In certain embodiments of the invention, expression of the recombinant polypeptide is regulatable, e.g., its expression is under control of a regulatable promoter. Suitable inducible or repressible promoters are known in the art. For example, certain promoters are inducible by heavy metals, hormones, small molecules, etc. Drug-regulatable promoters that are particularly well suited for use in mammalian cells include the tetracycline regulatable promoters, and glucocorticoid, sex hormone steroid-regulatable promoters. In one embodiment, a nucleic acid construct is provided that encodes both fusion proteins. Optionally the coding sequences are operably linked to separate promoters, e.g., in a vector. Optionally the coding sequences are under transcriptional control of the same promoter and are separated by an IRES sequence.

In some embodiments, the host cell is used as a source of polypeptides to perform a cell-free assay. In other embodiments, the assay is performed using the host cell, which is engineered to express both components of the reporter system. The host cell could be any cell type in which polypeptides can be expressed, e.g., prokaryotic (bacterial) or eukaryotic (e.g., yeast, insect, mammalian). In some embodiments the cells are human cells. One of skill in the art will be able to select appropriate expression vectors and expression control elements for expression of the fusion proteins in a cell type of choice. The fusion proteins may be tested to ensure that the RET polypeptide does not significantly inhibit the UNC93B/UNC93B-dependent TLR interaction and/or for any other functionally relevant activity. The cells may be transiently or stably transfected. Stable cell lines can be generated using standard selection methods. A cell has been “stably transfected” with a nucleic acid construct when the nucleic acid construct is capable of being inherited by daughter cells. “Transient transfection” refers to cases where exogenous DNA does not integrate into the genome of a transfected cell, e.g., where episomal DNA is transcribed into mRNA and translated into protein. Commonly used host cells include, but are not limited to, CHO, R1.1, B-W, L-M, COS-1, COS-7, BSC-1, BSC-40, BMT-10, BHK, HeLa, HEK-293, NIH/3T3, HT1080, HEK-293T, WI-38, and CV-1. For an extensive list of mammalian cell lines, those of ordinary skill in the art may refer to the American Type Culture Collection catalog (ATCC, Manassas, Va.).

In some embodiments, the cells do not naturally express UNC93B. In some embodiments, the cells do not naturally express an UNC93B-dependent TLR. In some embodiments, the cells are genetically modified to silence or knock out expression of UNC93B and/or one or more TLRs. In some embodiments knockout is achieved by deleting all or part of the gene encoding UNC93B or the TLR or otherwise disabling it. In some embodiments, silencing is achieved by engineering the cells to express an RNAi agent such as an shRNA that targets the UNC93B or TLR mRNA for degradation or otherwise silences expression. In other embodiments, the cells are contacted with an exogenous RNAi agent such as an siRNA.

In some embodiments, the assay is performed using an assay composition in which UNC93B and the TLR are present in association with a lipid bilayer. For example they may be attached to or span an endoplasmic reticulum (ER) endosomal or other cellular membrane or an artificial lipid bilayer. The membrane could be present in an intact cell or in a subcellular fraction isolated from the cell. It will be appreciated that in these embodiments the RET donor and acceptor moieties are desirably positioned so that they are both on the same side of the bilayer. For example, if the proteins are bound to or span an ER membrane, the RET donor and acceptor should be located either inside or outside the ER lumen. In certain embodiments, an inventive assay comprises isolating a subcellular fraction comprising membranes with which UNC93B and/or an UNC93B-dependent TLR is physically associated.

In some embodiments, the RET donor or acceptor is a nanoparticle or is physically associated with a nanoparticle. The particle may be covalently or noncovalently attached to an UNC93B polypeptide or a TLR polypeptide to form the first and second components of the proximity-dependent reporter system. The nanoparticles may be RET donors or acceptors or may have a RET donor or acceptor associated therewith. In some embodiments quantum dots are used. Quantum dots (QDs) are nanocrystals with physical dimensions small enough such that the effect of quantum confinement gives rise to unique optical and electronic properties that are not observed either in the bulk material, in discrete atoms, or in larger nanoparticles. Semiconductor QDs are often composed of atoms from groups II-VI or III-V in the periodic table. Examples include CdSe/ZnS-based QDs and InGaP/ZnS-based QDs. By varying the size and composition of the QDs, the emission wavelength can be adjusted in a predictable and controllable manner from the blue to the near infrared. QDs may be coated with a variety of different materials (e.g., lipids, PEG or other organic polymers, or combinations thereof) to improve their solubility in aqueous media. Quantum dots and methods for their synthesis and conjugation with biomolecules are well known in the art (see, e.g., Chan, W. C W. et al, Curr. Op. Biotech., 13, 40-46; 2002). QDs with a wide range of absorption and emission spectra are commercially available, e.g., from Evident Technologies (Troy, N.Y.) or Quantum Dot Corp. (Hayward Calif.; now owned by Invitrogen Corp.), etc. Suitable FRET donors or acceptors for QDs are readily identified.

Metal, nonmetal, and/or polymeric nanoparticles or nanoclusters are of use in the invention. Silica-containing particles or particles composed at least in part of organic polymers can be used. In one embodiment, the particles comprise an outer layer of silica and an inner metallic (e.g., comprised at least in part of a noble metal) or semiconductor (e.g., QD) core. Certain particles have sufficient optical transparency to allow efficient excitation of and/or emission of light from moieties located in their interior. A FRET donor can be associated with such a nanoparticle in a variety of ways. For example, the FRET donor can embedded or entrapped within the particle or covalently or noncovalently bound to its surface, etc.

RET donor and acceptor moieties may incorporate or be functionalized with a wide variety of functional groups (e.g., maleimide, succinimidyl esters (e.g., NHS), carbodiimmides, carbonylidiimidazoles, periodate, sulfonyl chloride), etc., to facilitate attachment to biomolecules such as polypeptides (e.g., via a primary amine, carboxyl, or sulfhydryl group of the polypeptide). One of skill in the art will be aware of many methods for attaching a detectable moiety to a polypeptide either directly or via a suitable linker, and guidance for doing so is readily available. See, e.g., Pierce Chemical Technical Library, available on the World Wide Web at piercenet.com; Wong S S, Chemistry of Protein Conjugation and Crosslinking, CRC Press Publishers, Boca Raton, 1991; and G. T. Hermanson, Bioconjugate Techniques, Academic Press, Inc., San Diego, 1996. Noncovalent attachment means could also be used. Such attachments are typically based on a specific high-affinity interaction such as a biotin-avidin or antibody-antigen interaction. An UNC93B or TLR polypeptide could be engineered to incorporate avidin or an epitope tag such as a hemagglutinin (HA), Myc, 6×-His, or FLAG tag, etc., or any other domain for which a high affinity binding partner exists. The RET donor or acceptor is modified to incorporate the appropriate binding partner, e.g., biotin or an antibody or other binding reagent that specifically binds to the tag. Polypeptides comprising an UNC93B polypeptide or UNC93B-dependent TLR polypeptide and an epitope tag have a variety of other uses in the practice of the inventive methods as described herein.

One of skill in the art will be familiar with appropriate assay conditions for performing a RET-based assay and detecting RET.

(2) Protein Fragment Complementation Assays

Protein fragment complementation assays (PCAs) for the detection of agents that modulate the interaction between UNC93B and an UNC93B-dependent polypeptide are provided. PCA involves the oligomerization and functional complementation of fragments of a reporter protein when the fragments are brought into close proximity with one another so that they are able to physically interact. A general strategy for implementing a PCA for the detection of agents that modulate the interaction between UNC93B and an UNC93B-dependent TLR is as follows: A protein (reporter protein) capable of directly or indirectly generating a detectable signal is selected. In the case of a monomeric reporter protein, the open reading frame encoding the protein is divided into first and second segments that encode polypeptide fragments capable of assembling to form a functional protein. “Functional” in this context means that the protein displays a detectable activity under suitable conditions and/or is capable of operating in its intended manner either alone or in combination with other assay component(s) to cause a detectable activity. The detectable activity could be production of a detectable signal and/or conferring a selectable or detectable phenotype on a cell. Standard molecular biology techniques are used to clone one of the segments in frame with a polynucleotide that encodes an UNC93B polypeptide and the other fragment in frame with a polynucleotide that encodes an UNC93B-dependent TLR polypeptide, resulting in first and second nucleic acid constructs that encode first and second polypeptides, wherein the first polypeptide comprises an UNC93B polypeptide fused to a first fragment of a reporter protein and the second polypeptide comprises an UNC93B-dependent TLR polypeptide fused to a second fragment of the reporter protein, wherein the first and second fragments are selected such that they are able to assemble to reconstitute a functional reporter protein when brought into close proximity with one another. In the case of a dimeric reporter protein, similar constructs are generated except the fragments are first and second polypeptide subunits of the protein rather than portions of a single polypeptide. Dimeric fluorescent proteins usable for PCAs or RET-based assays are described in U.S. Pat. No. 6,936,428. It will be appreciated that variant forms of the reporter proteins described herein could be used for the assays described herein.

The nucleic acid constructs are introduced into cells and expressed using standard techniques as described above. Typically the nucleic acid constructs are inserted into an expression vector, wherein they are operably linked to expression control sequences (e.g., promoter) capable of directing expression in a cell type of interest. Reassembly of the reporter protein from its fragments is facilitated by the binding of the UNC93B and TLR polypeptides to each other, which brings the fragments close together, allowing them to interact. Reconstitution is observed using a suitable assay. Observation of reconstituted protein functional activity should provide a measure of the interaction of the fused proteins. Thus assay compositions for identification of agents that modulate the interaction between UNC93B and an UNC93B-dependent TLR are provided. In some embodiments, the composition comprises (i) a first complementary fragment of a reporter protein fused to an UNC93B polypeptide, and (ii) a second complementary fragment of a reporter protein fused to an UNC93B-dependent TLR polypeptide, wherein the complementary fragments exhibit a detectable activity when associated. Optionally, the composition further comprises (a) a candidate agent; (b) a ligand for the TLR; (c) a substrate of the reporter protein, or any combination of (a)-(c). In one embodiment, the assay composition is inside a living cell. In another embodiment, the assay composition comprises isolated polypeptides. In another embodiment, the assay composition is formed by combining substantially purified polypeptides. In some embodiments, the polypeptides are found in their native configuration in a cellular membrane.

Any of a wide variety of proteins that have a detectable activity can be used as reporter proteins. Examples include various enzymes, chromoproteins, fluorescent, luminescent, or phosphorescent proteins, selectable markers, etc. As used herein, the terms chromoprotein and fluorescent protein refer to any protein that is pigmented or colored and/or fluoresces when irradiated with light, e.g., white light or light of a specific wavelength (or narrow band of wavelengths such as an excitation wavelength). See Pelletier, J. N., et al., J. Biomolecular Techniques 10:32, 1998; U.S. Pat. Nos. 6,270,964; 6,294,330; 6,828,099. See U.S. Pat. No. 7,166,424, which describes a number of suitable fragments of fluorescent or luminescent polypeptides. See also U.S. Pat. No. 7,166,444, which describes additional chromo/fluoroproteins of use. Enzymes of use include dihydrofolate reductase (DHFR), beta-lactamase, enzymes encoding any selectable nutritional or drug resistance marker or any enzyme capable of acting on a substrate to produce a detectable e.g., colorimetric, fluorescent, or luminescent signal.

In one embodiment, the reporter protein is a protein involved in intracellular protein degradation (e.g., ubiquitin), and the fragments are inactive fragments that can reassemble to form a functional protein. For purposes of description it will be assumed that the protein is ubiquitin (Ub). Ubiquitin plays a role in a number of processes primarily through routes that involve protein degradation. In eukaryotes, newly formed Ub fusions are rapidly cleaved by ubiquitin specific proteases (UBPs) after the last residue of Ub at the Ub-polypeptide junction. The cleavage of a Ub fusion by UBPs requires the folded conformation of Ub. When a C-terminal fragment of ubiquitin is expressed as a fusion to a reporter protein, the fusion is cleaved only if an N-terminal fragment of Ub is also expressed in the same cell and can assemble with the C-terminal fragment. In one embodiment the assay composition comprises (i) a first fusion protein comprising an UNC93B polypeptide and a first fragment of ubiquitin (e.g., an N-terminal domain), and (ii) a second fusion protein comprising an UNC93B-dependent TLR and a second fragment of ubiquitin (e.g., a C-terminal domain) followed by a transcriptional activator. In another embodiment, the assay composition comprises (i) a first fusion protein comprising an UNC93B-dependent TLR polypeptide and a first fragment of ubiquitin (e.g., an N-terminal domain), and (ii) a second fusion protein comprising an UNC93B polypeptide and a second fragment of ubiquitin (e.g., a C-terminal domain) followed by a reporter polypeptide that has a detectable activity when released from the ubiquitin fragment but not when fused to the ubiquitin fragment. Interaction of the UNC93B-dependent TLR and the TLR brings the two fragments of ubiquitin into close proximity, facilitating their reconstitution into an active ubiquitin. Ubiquitin-specific proteases (UBPs), which are present in all eukaryotic cells, recognize the reconstituted ubiquitin, but not its fragments, and cleave the ubiquitin off the reporter polypeptide. An increase in the activity of the reporter protein, which may be detected in any suitable manner depending on the identity of the reporter, is indicative of an interaction between the UNC93B-dependent TLR and the UNC93B polypeptide. In one embodiment, the reporter polypeptide is or comprises a transcriptional activator or repressor operably linked to a polypeptide domain that binds to a defined nucleic acid sequence (regulatory sequence). The assay composition further comprises a transcriptional reporter construct containing the regulatory sequence operably linked to a nucleic acid sequence encoding a second reporter protein, such that a second reporter protein having a detectable activity is produced when transcription is activated by the transcriptional activator. The second reporter protein may be any of the reporter proteins discussed above. The transcriptional reporter construct optionally comprises a minimal promoter in addition to the binding site for the transcriptional activator. The method is performed in cells of suitable cell type, e.g., yeast or mammalian cells, that contains or is manipulated to contain a ubiquitin-specific protease capable of recognizing the ubiquitin expressed by the cells. In certain embodiments, the transcriptional activator is one that does not naturally occur in the cells used in the assay, e.g., it is from a different species or is an artificial transcriptional activator. Exemplary ubiquitin fragments, reporter constructs, binding sites and polypeptide domains capable of binding thereto, etc., are described, e.g., in U.S. Pat. Pub. No. 20050106636. See also, U.S. Pat. Nos. 5,585,245; 5,503,977; and U.S. Pat. Pub. No. 20040170970 which further describe “split ubiquitin” methods. In one embodiment, either or both of the fragments is mutated such that reconstitution only occurs when the fragments are fused to polypeptides that interact with one another. In one embodiment, the polypeptide domain is a LexA repressor.

It will be appreciated that many variations of this assay approach are encompassed by the invention. For example, a transcriptional repressor could be used, wherein a reduction in the amount of the reporter protein is indicative of an interaction between the UNC93B-dependent TLR and the UNC93B polypeptide. The polypeptide having a detectable activity when released from the protease fragment or ubiquitin fragment could be fused to an N-terminal fragment of the protease or ubiquitin rather than a C-terminal fragment. In certain embodiments, the fragments are fragments of a protease that has a known cleavage site. The assay composition comprises a transcriptional activator that is attached to a polypeptide located outside the nucleus via a sequence comprising the cleavage site. Assembly of the protease from its fragments results in cleavage of the cleavage site, thereby releasing the transcriptional activator and allowing it to enter the nucleus and activate transcription of a second reporter protein. In certain embodiments, the protease is one that does not naturally exist in the cells used in the assay.

In another embodiment, the reporter protein is one that activates the unfolded protein response pathway. See, e.g., U.S. Pat. Pub. Nos. 20020160408 and 20050032197. The invention provides assay compositions based on either of the afore-mentioned disclosures, wherein the assay composition is adapted to identify agents that modulate physical interaction of UNC93B polypeptide and an UNC93B-dependent TLR and/or to identify additional polypeptide(s) that physically interact with a complex comprising UNC93B and an UNC93B-dependent TLR. In some aspects, assay compositions comprising polypeptides in which an UNC93B-dependent TLR polypeptide is fused to at least a portion of an Ire1p polypeptide and an UNC93B polypeptide is fused to at least a portion of an Ire1p polypeptide are provided. Physical interaction of the polypeptides results in dimerization of the Ire1p portions, thereby activating a reporter system that may be based on the unfolded protein response pathway.

It will be appreciated that a number of considerations relevant to assays based on RET are applicable to the PCAs of the invention. For example, in some embodiments, the assay is performed using a composition in which UNC93B polypeptide and the TLR polypeptide are present in association with a lipid bilayer. For example, they may be attached to or span an endoplasmic reticulum (ER) endosomal or other cellular membrane or an artificial lipid bilayer. The membrane could be present in an intact cell or in a subcellular fraction isolated from the cell. It will be appreciated that in these embodiments the first and second protein fragments are desirably positioned so that they are both on the same side of the bilayer. For example, if the proteins are bound to or span an ER membrane, the fragments should be located either inside or outside the ER lumen. In certain embodiments an inventive assay comprises isolating a subcellular fraction comprising membranes with which UNC93B and/or an UNC93B-dependent TLR is physically associated.

One of skill in the art will be familiar with appropriate assay conditions for performing a PCA and detecting activity of particular reporter proteins.

(3) Proximity Dependent Reporter Systems Based on Plasmon Resonance

Surface plasmon resonance (SPR) is an optical phenomenon that can be used to measure changes in the solution concentration of molecules at a biospecific surface. The signal arises in thin metal films under conditions of total internal reflection and depends on the refractive index of solutions in contact with the surface. Molecules in solution exhibit changes in refractive index and thus give rise to a measurable SPR signal if a biospecific interaction occurs. Typically, protein or DNA is immobilized by one of several possible methods onto a carboxymethylated dextran-gold surface. The interacting protein of interest is injected over the surface and the kinetics of binding are measured in real time. Instruments suitable for quantifying protein-protein interactions using SPR are available from BIAcore. In some embodiments, such a system is used to perform secondary assays on candidate agents initially identified using either an interaction-specific assay or an interaction-independent assay rather than for initial screening of a large number of candidate agents.

(4) Assays to Detect Agents Capable of Modulating Interaction Between UNC93B and Multiple UNC93B-Dependent TLRs

Interaction-specific assays can be performed using assay compositions that contain any 1, 2, 3, or more UNC93B-dependent TLRs. Individual assays could be performed to determine whether an agent modulates the interaction between UNC93B polypeptide and all of the UNC93B-dependent TLRs or a subset thereof. For example, a first assay could determine whether an agent modulates the interaction between UNC93B polypeptide and a TLR3 polypeptide; a second assay could determine whether the same agent modulates the interaction of UNC93B polypeptide and TLR7 polypeptide, etc., Agents that modulate physical interaction between UNC93B polypeptide and any 1, 2, 3, or more TLRs of interest may thus be identified. Any of the interaction-specific assays can be adapted to identify agents that modulate the interaction of UNC93B polypeptide and any one or more UNC93B-dependent TLRs. For example, a RET-based assay composition could comprise following three components: (i) a first polypeptide comprising an UNC93B polypeptide having a RET donor or acceptor physically associated therewith; (ii) a second polypeptide comprising a first UNC93B-dependent TLR polypeptide having a RET acceptor or donor physically associated therewith; and (iii) a third polypeptide comprising a second UNC93B-dependent TLR physically associated therewith. The first and second TLRs are different. It will be appreciated that if the UNC93B polypeptide has a RET donor associated therewith, then the TLR polypeptides should have a RET acceptor associated therewith, while if the UNC93B polypeptide has a RET acceptor associated therewith, then the TLR polypeptides should have a RET donor associated therewith. The assay is performed as described above. Agents that, for example, inhibit the interaction between UNC93B and both TLRs will be distinguished from agents that do not inhibit either interaction or inhibit only one interaction. The assay could utilize any 2, 3, or more TLRs. The RET moiety could be the same or different for each TLR. Similarly, the PCA assay compositions could comprise multiple TLR polypeptides, each physically associated with a fragment of a reporter protein capable of assembling with the fragment physically associated with the UNC93B polypeptide to reconstitute a functional reporter protein. Assays performed using such compositions would distinguish agents that inhibit interaction of UNC93B with all of the represented TLRs from those that inhibit only one or none of the interactions.

(5) Localization-Based Assays

The endosomal compartment comprises a network of membranous tubes and vesicles extending from just beneath the cell membrane (early endosomes) to close to the Golgi and near the periphery of the nucleus (late endosomes). The interior of the endosomal compartment is acidic (pH ˜6), with later endosomes typically more acidic than early endosomes. Lysosomes are membrane-bound organelles that contain digestive enzymes (acid hydrolaseses) and generally have a lower internal pH than endosomes (−4.8). Parts of the late endosomal compartment may become lysosomes or fuse with lysosomes, forming endolysosomes. Phagosomes are large endocytic vesicles containing internalized (phagocytosed) particulate materials such as microorganisms, dead cells and cellular debris, etc. Phagosomes often fuse with lysosomes inside the cell, and the ingested material is then degraded. Thus cells can contain vesicles derived from fusion of late endosomes with lysosomes, lysosomes with phagosomes, etc. For purposes of the present invention, late endosomes, lysosomes, and phagosomes will be referred to as the “endosomal/lysosomal/phagosomal (ELP) compartment”.

Following stimulation, UNC93B-dependent TLRs translocate to the endolysosomal compartment, which contains a variety of molecules involved in TLR signaling. Applicants discovered that UNC93B delivers nucleotide-sensing TLRs from the ER to the ELP compartment and that endosomal/endolysosomal localization is essential for normal signaling of nucleotide-sensing TLRs. As described in further detail in Examples 9-14, Applicants showed that UNC93B delivers the nucleotide-sensing TLRs TLR 7 and 9 from the ER to endolysosomes. To Applicants' knowledge, UNC93B is the first protein to be identified as a molecule having this activity. Assessing localization of UNC93B and/or localization of an UNC93B-dependent TLR such as TLR 7 or 9 can be used to determine whether an agent is capable of modulating interaction between UNC93B and UNC93B-dependent TLR(s). It can also be used to determine whether an agent is capable of inhibiting UNC93B-dependent TLR(s) from transmitting signals by blocking delivery of UNC93B-dependent TLR(s) to the ELP compartment. In the absence of a stimulus, UNC93B and UNC93B dependent TLRs are localized to the ER. However, upon stimulation (e.g., upon exposure to a ligand for the TLR), UNC93B and UNC93B dependent TLRs responsive to the stimulus translocate to the endosomal/lysosomal/phagosomal compartment. Translocation of the TLRs is at least in part dependent on interaction with UNC93B.

The invention provides a variety of assays for identifying agents that modulate the UNC93B/TLR interaction, which are based at least in part on detecting differences in localization of UNC93B and/or UNC93B-dependent TLR(s) in cells that have been contacted with such agents versus cells that have not been contacted with the agent (or have been contacted with lower concentrations of the agent). Cells and recombinant polypeptides useful in performing the assays, and kits containing them, are also an aspect of the invention. For example, the invention provides a method of identifying an immunomodulator that modulates signaling by an UNC93B dependent TLR, comprising the steps of: (a) contacting a cell that expresses both an UNC93B polypeptide and an UNC93B-dependent TLR polypeptide with a candidate agent; and (b) determining whether the candidate agent affects (e.g., changes, modulates) the localization of the UNC93B polypeptide or the TLR polypeptide, wherein an agent that affects the localization of the UNC93B polypeptide or the TLR polypeptide is identified as a potential immunomodulator that modulates signaling by an UNC93B-dependent TLR. In some embodiments of the method, the cell is contacted with a stimulus for the TLR signaling pathway, e.g., a stimulus that would be expected to result in translocation of UNC93B and the TLR from the ER to the ELP under normal conditions (e.g., under standard culture conditions for cells of that type and in the absence of the candidate agent). The effect of the agent on localization is assessed. In accordance with the invention an agent that inhibits the UNC93B/TLR interaction is expected to result in an increased amount of UNC93B and UNC93B-dependent TLRs in the ER relative to the ELP compartment following a stimulus, as compared with the amount of UNC93B and UNC93B-dependent TLR that would be present in the ER relative to the ELP following the stimulus in the absence of the agent. Thus an increased ER/ELP ratio (or decreased ELP/ER ratio) is indicative of an agent that inhibits UNC93B/TLR interaction. An agent that enhances the UNC93B/TLR interaction is expected to result in an increased amount of UNC93B and UNC93B-dependent TLRs in the ELP compartment relative to the ER following a stimulus, as compared with the amount of UNC93B and UNC93B-dependent TLR that would be present in the ELP compartment relative to the ER following the stimulus in the absence of the agent. Thus an increased ELP/ER ratio (or decreased ER/ELP ratio) is indicative of an agent that enhances UNC93B/TLR interaction. In some embodiments of the invention differences in localization to the endosomal compartment are used. In some embodiments of the invention differences in localization to the endolysosomal compartment are used. In some embodiments of the invention differences in localization to the lysosomal compartment are assessed. In some embodiments of the invention differences in localization to the phagosomal compartment are assessed. In some embodiments of the invention differences in localization to any two, or all three compartments are assessed.

In some embodiments of the invention the UNC93B polypeptide and/or the TLR polypeptide is detectably labeled. The polypeptide is considered detectably labeled if its sequence is modified relative to the naturally occurring sequence in a manner that renders it more readily detectable. For example, the sequence can be modified to include a fluorescent or luminescent protein domain such as those mentioned above as reporter proteins (e.g., GFP, RFP, BFP, YFP, CYP, SFP, reef coral fluorescent protein, mCherry, luciferase, aequorin and derivatives of these), thereby rendering it directly optically detectable. For example, a recombinant polypeptide such as TLR9-GFP or UNC93B-mCherry could be used. In certain embodiments of the invention the detectable domain (e.g., GFP) is at the C-terminus. The sequence can be modified to include an epitope tag (e.g., Myc tag, HA tag, etc.) or other domain to which antibodies or other binding agents are readily available, thereby rendering the polypeptide more readily detectable. For example, the sequence can be modified to include a domain to which detectably labeled (e.g., radioactively labeled, fluorescently or enyzmatically labeled, metal-labeled) antibodies are available. Alternately, a detectably labeled secondary antibody is used to detect the primary antibody. Cells to be used in the assay may be transfected with a nucleic acid construct encoding the modified UNC93B or TLR polypeptide. Since UNC93B and UNC93B-dependent TLRs colocalize, the assay may include detecting UNC93B, an UNC93B-dependent TLR, or both. Other modifications include incorporating polypeptides sequences whose fluorescence properties are altered upon encountering a lower pH in the ELP compartment. It will be appreciated that such polypeptides must be properly located so as to be inside the compartment. Endogenous unmodified UNC93 and/or TLR polypeptides can be detected, e.g., using antibodies or other specific binding agents that are detectably labeled or are themselves detected using appropriately labeled secondary antibodies. Such methods are known in the art and are exemplary of the methods that can be used to detect UNC93B and/or TLRs.

The ELP compartment can be selectively labeled, detected, and distinguished from other intracellular compartments, and the various membrane-bound organelles of which the ELP compartment is composed can be distinguished from each other. Similarly, the ER can be selectively labeled, detected and distinguished from other organelles such as those of the ELP compartment. Detection can be accomplished by detecting various molecules that accumulate preferentially in particular organelle(s) and/or whose properties change in a detectable manner upon entering such organelle(s), e.g., as a result of experiencing an altered pH. Certain cellular polypeptides accumulate preferentially in one or more of these organelle types and are typically found therein. Certain polypeptide domains direct polypeptides containing them to particular organelles and/or cause retention of polypeptides containing them within particular organelles. Polypeptides that are found selectively and characteristically (at least in the cell type of interest) within particular organelle(s) or at particular locations are referred to collectively herein and in the art as “markers” or “marker polypeptides” for these organelles or locations. Such polypeptides may contain a sorting sequence (sorting motif) or retention sequence (retention motif), which directs the polypeptide to a compartment/location in the cell and/or causes it to be retained therein, sometimes at least in part via interactions with other proteins. For example, ER resident proteins are retained in the endoplasmic reticulum through a retention motif such as the KDEL (SEQ ID NO: 13) sequence. Polypeptides containing such motifs can be used as markers for the ER. The polypeptide could comprise or consist of KDEL (SEQ ID NO: 13) and a detectable polypeptide domain or tag (e.g., GFP-KDEL or mCherry-KDEL). Markers for the ER include Binding immunoglobulin protein (BiP; also called glucose regulated protein 78, Grp78, and Heat shock protein 5) (Gene ID 14828 (mouse); 3309 (human)), SEL1L (Gene ID 20338 (mouse); 6400 (human); Derlin-1 (Gene ID 79139 (mouse); 67819 (human)), Derlin-2 116891 (mouse); 51009 (human); calnexin (Gene ID 12330 (mouse); 821 (human)), all of which are well known in the art. Markers for the endolysosomal compartment include lysosome associated membrane protein one (Lamp1; Gene ID 16783 (mouse); 3916 (human)), CD63 (Gene ID 12512 (mouse); 967 (human), Lamp2; Gene ID 16784 (mouse); 3920 (human)). Early endosome antigen 1 (EEA1; Gene ID 216238 (mouse); 8411 (human)) or Rab5a (Gene ID 271457 (mouse); 5868 (human)) can be used as an early endosomal marker Rab7 (Gene ID 19349 (mouse); 7879 (human)) is a late endosomal marker. Thus a recombinant polypeptide such as Lamp1-mCherry or CD63-GFP could be used. One of skill in the art will be aware of additional markers. It will be understood that marker polypeptides need not be found exclusively within those organelles for which they are considered to be markers. It will also be understood that the sequence of a marker polypeptide may vary from the reference sequences available in databases, provided that the variation does not substantially affect localization. Either naturally occurring or engineered variants could be used. In some embodiments a sorting sequence or retention sequence fused to a detectable polypeptide is used as a marker polypeptide.

The marker polypeptide may be detectably labeled by incorporating a fluorescent or luminescent domain, readily detectable epitope tag, etc. In certain embodiments of the invention the detectable domain (e.g., GFP) is at the C-terminus. Cells to be used in the assay may be transfected with a nucleic acid construct encoding the modified marker polypeptide. In some embodiments of the invention endogenous unmodified marker polypeptides are used. Such polypeptides can be detected using antibodies or other specific binding agents that are detectably labeled or are themselves detected using appropriately labeled secondary antibodies. Such methods are known in the art and are exemplary of the methods that can be used to detect the ELP or ER. Phagosomes can be labeled, e.g., by contacting the cell with particulate material that is detectable following phagocytosis. For example, appropriately sized polymeric beads can be used. Bacteria or yeast can be used as the particulate material. Optionally, the particulate material comprises an optically detectable substance such as a fluorescent dye or quantum dots. Molecules that accumulate selectively or specifically in the ER or in the ELP compartment upon being taken up by cells and/or undergo a detectable change in their properties upon entering such compartment may be used as markers to label the ER or ELP. Some of these molecules are not directly detectable but are typically rendered detectable using antibodies conjugated to a directly or indirectly detectable moiety such as fluorophore or enzyme, while others are fluorescent or colored and therefore intrinsically optically detectable. They may be referred to as “dyes”, “stains”, or “indicators”. Both classes of marker molecules are considered “detectably labeled for purposes of this invention.” For example, ER-Tracker™ dyes (Invitrogen, Carlsbad, Calif.) are cell-permeant, live-cell stains that are highly selective for the ER. ER-Tracker Blue-White DPX is a member of the Dapoxyl™ dye family. ER-Tracker™ Green and ER-Tracker™ Red dyes are the drug conjugates glibenclamide BODIPY® TL and glibenclamide BODIPY® TR, respectively. Carbocyanine dyes such as DiOC₆ are additional ER stains.

Weakly basic amines selectively accumulate in cellular compartments with low internal pH such as lysosomes and can be used to selectively label such compartments. One frequently used probe for acidic organelles, DAMP (Invitrogen Cat. No. D1552), is not fluorescent and is therefore usually used in conjunction with anti-DNP antibodies directly or indirectly conjugated to a fluorophore or enzyme in order to visualize the staining pattern. The fluorescent probes neutral red (N3246) and acridine orange (Invitrogen Cat. Nos, A1301, A3568) are also commonly used for staining acidic organelles, though they lack specificity. The LysoTracker® probes are fluorescent acidotropic probes useful for labeling and tracing acidic organelles in live cells. These probes possess high selectivity for acidic organelles and effectively label live cells at nanomolar concentrations. The LysoTracker probes, which comprise a fluorophore linked to a weak base that is only partially protonated at neutral pH, are freely permeant to cell membranes and typically concentrate in spherical organelles. LysoSensor® dyes are acidotropic probes that appear to accumulate in acidic organelles as the result of protonation. This protonation also relieves the fluorescence quenching of the dye, resulting in an increase in fluorescence intensity. Thus, the LysoSensor probes exhibit a pH-dependent increase in fluorescence intensity upon acidification, in contrast to the LysoTracker probes, which exhibit fluorescence that is not substantially enhanced at acidic pH. A variety of different absorption and emission maxima facilitating simultaneous imaging of multiple markers. Further details regarding these and other markers for the ER or endosomal/lysosomal/phagosomal compartments are found in “Handbook of Fluorescent Probes and Research Products” (Molecular Probes, 9^{th} edition, 2002) and/or “The Handbook—A Guide to Fluorescent Probes and Labeling Technologies”, (Invitrogen, 10th edition, available at the Invitrogen web site—www.intvitrogen.com/handbook, see Ch. 12 in particular).

In order to carry out the method, cells are contacted with a candidate agent. “Contacting” can be performed by placing the agent and cells into a liquid medium (e.g., tissue culture medium) in which the cells are cultured. The cells are assessed to determine whether exposure to the agent affects localization of UNC93B and/or an UNC93B-dependent TLR, e.g., upon stimulating the cells with a ligand for the TLR. In some embodiments, the ELP and/or ER are detected, and the relative amount of UNC93B in each compartment is determined. In some embodiments, the ELP and/or ER are detected, and the relative amount of a TLR in each compartment is determined. In some embodiments, both ELP and ER are detected. By using distinguishable labels (e.g., fluorescent moieties with differing emission peaks), these compartments can be distinguished from each other in the same cells, if desired. However, it is not necessary to use the same population of cells to compare ER and ELP localization of UNC93B or TLR. Instead, two separate populations of cells could be exposed to the agent. One population would be used to assess ER localization and the second population would be used to assess ELP localization. The UNC93B or TLR polypeptide may be labeled with a label that allows its detection when present within a labeled ER or ELP compartment. For example, if TLR is labeled “green” and endolysosomes are labeled “red”, superimposing images showing “green” and “red” signals will result in “yellow” where TLR is located in endolysosomes. If localization of UNC93B and/or the TLR is different in cells that have been exposed to the candidate agent than in cells not exposed to the agent (or cells exposed to a lower or higher concentration of the agent), the candidate agent affects UNC93B/TLR localization and, potentially, the UNC93B/TLR interaction. Exposure to the agent can be transient or can continue throughout the duration of the assay. Secondary assays can be performed, if desired, to determine whether an agent identified as affecting UNC93B/TLR localization selectively or specifically affects UNC93B/TLR localization, affects localization of other proteins as well, or has other effects such as disrupting retention of KDEL-containing proteins in the ER or biogenesis of endosomes/lysosomes. Additional tests can be performed to determine whether the agent affects signaling by non-UNC93B dependent TLRs. In any of the embodiments of the invention a candidate agent identified as an immunomodulator may be subjected to additional cell-based and/or in vivo tests to further define and/or confirm its activity.

C. Detection

A variety of different detection systems can be used and can comprise part of an assay system of the invention. One of skill in the art will be able to select an appropriate detection system based on the particular characteristics of the assay and the detectable signal. Enzymes may be visually detected by a colorimetric signal produced by their activity on a suitable substrate. Fluorescence can be measured using a fluorometer or by fluorescence activated cell sorting. Luminescence can be measured using a luminometer. Microplate readers, scanning spectroscopy, and microscopes coupled to charge-coupled device (CCD) cameras are of use. Suitable instrumentation systems have been developed to automate screens based on detection of signals from intact cells, including the automated fluorescence imaging and automated microscopy systems developed by Cellomics, Amersham, TTP, Q3DM, Evotec, Universal Imaging and Zeiss. Assay systems comprising such detection apparatus for detecting a signal produced by, e.g., emanating from, an assay composition are an aspect of the invention. “Detecting a signal produced by said assay composition” includes detecting an alteration or absence of signal. Furthermore, it is understood that an assay composition may require an energy input or substrate in order to produce a signal. Fluorescence microscopy (e.g., fluorescence confocal microscopy) is a suitable means for detecting localization of UNC93B or an UNC93B dependent TLR and for assessing whether UNC93B/TLR colocalizes with ER or endosomal/phagosomal/lysosomal markers(s). Quantitative colocalization analyses can be performed with suitable software, e.g., ‘Measure Colocalization’ and ‘Correlation Plot’ in the MetaMorph software (Molecular Devices).

D. Candidate Agents and Screening Formats

A variety of different candidate agents can be tested to determine whether they alter binding of UNC93B to a TLR and/or modulate TLR-mediated signaling. A candidate agent can be any molecule or supramolecular complex, e.g. polypeptides, peptides (which is used herein to refer to a polypeptide consisting of 60 amino acids or less), small organic or inorganic molecules (i.e., molecules having a molecular weight less than 1,500 Da, 1000Da, or 500 Da in size), polysaccharides, polynucleotides, etc. which is to be tested for ability to modulate TLR signaling and/or an UNC93B-TLR interaction. In some embodiments, the candidate agents are organic molecules, particularly small organic molecules, comprising functional groups that mediate structural interactions with proteins, e.g., hydrogen bonding, and typically include at least an amine, carbonyl, hydroxyl or carboxyl group, and in some embodiments at least two of the functional chemical groups. The candidate agents may comprise cyclic carbon or heterocyclic structures and/or aromatic or polyaromatic structures substituted with one or more chemical functional groups and/or heteroatoms.

Candidate agents are obtained from a wide variety of sources, as will be appreciated by those in the art, including libraries of synthetic or natural compounds. A variety of methods for screening any library of candidate modulators, including the wide variety of known combinatorial chemistry-type libraries are provided.

In some embodiments, candidate agents are synthetic compounds. Numerous techniques are available for the random and directed synthesis of a wide variety of organic compounds and biomolecules. In some embodiments, the candidate modulators are provided as mixtures of natural compounds in the form of bacterial, fungal, plant and animal extracts, fermentation broths, etc., that are available or readily produced. In some embodiments, a library of compounds is screened. The term “library of compounds” is used consistently with its usage in the art. A library is typically a collection of compounds that can be presented or displayed such that the compounds can be identified in a screening assay. In some embodiments compounds in the library are housed in individual wells (e.g., of microtiter plates), vessels, tubes, etc., to facilitate convenient transfer to individual wells or vessels for contacting cells, performing cell-free assays, etc. The library may be composed of molecules having common structural features which differ in the number or type of group attached to the main structure or may be completely random. Libraries include but are not limited to, for example, phage display libraries, peptide libraries, polysome libraries, aptamer libraries, synthetic small molecule libraries, natural compound libraries, and chemical libraries. Methods for preparing libraries of molecules are well known in the art and many libraries are available from commercial or non-commercial sources. Libraries of interest include synthetic organic combinatorial libraries. Libraries, such as, synthetic small molecule libraries and chemical libraries can comprise a structurally diverse collection of chemical molecules. Small molecules include organic molecules often having multiple carbon-carbon bonds. The libraries can comprise cyclic carbon or heterocyclic structure and/or aromatic or polyaromatic structures substituted with one or more functional groups. In some embodiments the small molecule has between 5 and 50 carbon atoms, e.g., between 7 and 30 carbons. In some embodiments the compounds are macrocyclic. Libraries of interest also include peptide libraries, randomized oligonucleotide libraries, and the like. Libraries can be synthesized of peptoids and non-peptide synthetic moieties. Such libraries can further be synthesized which contain non-peptide synthetic moieties which are less subject to enzymatic degradation compared to their naturally-occurring counterparts. Small molecule combinatorial libraries may also be generated. A combinatorial library of small organic compounds may comprise a collection of closely related analogs that differ from each other in one or more points of diversity and are synthesized by organic techniques using multi-step processes. Combinatorial libraries can include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Pat. No. 5,712,171. In some embodiments mixtures containing two or more compounds, extracts or other preparations obtained from natural sources (which may comprise dozens of compounds or more), and/or inorganic compounds, etc., are screened.

In one embodiment, the methods of the invention are used to screen “approved drugs”. An “approved drug” is any compound (which term includes biological molecules such as proteins and nucleic acids) which has been approved for use in humans by the FDA or a similar government agency in another country, for any purpose. This can be a particularly useful class of compounds to screen because it represents a set of compounds which are believed to be safe and, at least in the case of FDA approved drugs, therapeutic for at least one purpose. Thus, there is a high likelihood that these drugs will at least be safe for other purposes.

Natural or synthetically produced libraries and compounds are readily modified through conventional chemical, physical and biochemical means. Chemical (including enzymatic) reactions may be done on the moieties to form new substrates or candidate agents which can then be tested using the present invention. Known pharmacological agents may be subjected to directed or random chemical modifications, including enzymatic modifications, to produce structural analogs.

In some embodiments, candidate agents include peptides, nucleic acids, and chemical moieties. In one embodiment, the candidate modulators are naturally occurring polypeptides or fragments of naturally occurring polypeptides, e.g., from bacterial, fungal, viral, and mammalian sources. In one embodiment, the candidate modulators are nucleic acids of from about 2 to about 50 nucleotides, e.g., about 5 to about 30 or about 8 to about 20 nucleotides in length. In one embodiment, the candidate modulators are peptides of from about 2 to about 60 amino acids, e.g., about 5 to about 30 or about 8 to about 20 amino acids in length. The peptides may be digests of naturally occurring polypeptides or randomly synthesized peptides that may incorporate any amino acid at any position. In one embodiment a synthetic process can generates randomized polypeptides or nucleic acids, to allow the formation of all or most of the possible combinations over the length of the sequence, thus forming a library of randomized candidate bioactive agents. For example, a library of all combinations of amino acids that form a peptide 7 to 20 amino acids in length could be used. In one embodiment, the library is fully randomized, with no sequence preferences, constraints, or constants at any position. In one embodiment, the library is biased, i.e., some positions within the sequence are either held constant, or are selected from a limited number of possibilities. For example, the nucleotides or amino acid residues may be randomized within a defined class, for example, of hydrophobic, hydrophilic, acidic, or basic amino acids, sterically biased (either small or large) residues, towards the creation of cysteines for cross-linking, prolines for turns, serines, threonines, tyrosines or histidines for phosphorylation sites, etc. The peptides could be cyclic or linear.

In some embodiments, a collection of peptides that represent fragments of UNC93B or an UNC93B-dependent TLR are screened. UNC93B peptides may compete with full length UNC93B for binding to an UNC93B-dependent TLR. TLR peptides may compete with full length TLR for binding to UNC93B. In one embodiment the peptides comprise all or a portion of the transmembrane domain of UNC93B or a TLR. In one embodiment, the peptide is a fragment of UNC93B that includes residue 412 and at least one amino acid on either side of residue 412, e.g., amino acids 410-415, 408-415, 405-418, etc. The peptides are, e.g., between 5 and 30 amino acids in length.

It will be appreciated that naturally occurring nucleotides (e.g., A, G, C, T, U) or synthetic nucleotide analogs (e.g., having modifications of the base and/or sugar) and/or nucleic acids with modified nucleic acid backbone structures could be used. Many such compounds are known in the art. The invention contemplates use of nucleic acids having any of the modifications known in the art to be useful for improving stability (e.g., enhancing nuclease resistance), bioavailability, cell uptake, activity, etc., of nucleic acids such as aptamers and siRNAs. Amino acids could be naturally occurring amino acids or non-naturally occurring amino acids. If naturally occurring they may, but need not be, amino acids naturally found in proteins. The library could be based on a small molecule scaffold or protein domain. A number of molecules or protein domains are suitable as starting points for the generation of biased randomized candidate modulators. A large number of small molecule domains are known, that confer a common function, structure or affinity.

Representative examples of libraries that could be screened include DIVERSet™, available from ChemBridge Corporation, 16981 Via Tazon, San Diego, Calif. 92127. DIVERSet contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., Am. Chem. Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. For example, libraries based on quinic acid and shikimic acid, hydroxyproline, santonin, dianhydro-D-glucitol, hydroxypipecolinic acid, andrographolide, piperazine-2-carboxylic acid based library, cytosine, etc., are commercially available.

High throughput screening (HTS) methods, including ultra-HTS, are of use. Such methods typically employ microplates, microplate readers, and robotics to screen hundreds or thousands of compounds. In some embodiments a homogeneous assay, in which the entire assay is performed without need to transfer the sample from one vessel to another, is used. In some embodiments the assay makes use of automated digital microscopy and/or flow cytometry to evaluate individual cells. Candidate agents may be contacted with cells by adding the agent in a soluble form into an assay system containing cells, such as a culture vessel, microwell dish, etc. It will be appreciated that a candidate agent is present in addition to components normally present in the medium or composition in which the cells are maintained.

### IV. Complex Comprising an UNC93B Polypeptide and an UNC93B-Dependent TLR and Uses Thereof

Isolated complexes comprising an UNC93B polypeptide and an UNC93-dependent TLR polypeptide are provided. In some embodiments, the UNC93B polypeptide and the TLR polypeptide are wild type. In some embodiments, the UNC93B polypeptide, the TLR polypeptide, or both, further comprises a moiety selected from the group consisting of: (a) an epitope tag; (b) a fragment of a reporter protein; and (c) a RET donor or acceptor. In some embodiments, the UNC93B polypeptide or the TLR polypeptide is attached covalently or noncovalently to a solid support. Solid supports include, e.g., particles (e.g., agarose, sepharose, organic polymer, silica, plastic, metal), membranes (e.g., nitrocellulose or nylon), a microwell dish (e.g., PVC, polypropylene, or polystyrene), a microfuge or test tube (glass or plastic). In one embodiment, the particle is a chromatography resin or magnetic bead.

Methods of isolating a complex comprising an UNC93B polypeptide and an UNC93B-dependent TLR polypeptide are provided. In some embodiments, the methods comprise steps of: (a) maintaining cells that express UNC93B polypeptide and an UNC93B-dependent TLR polypeptide; (b) lysing the cells; and (c) isolating the complex under conditions consistent that do not substantially disrupt the complex. Such conditions are ones that preserve at least 10%, in some embodiments at least 25%, at least 50%, or at least 75% of the complexes. Methods of use to isolate the complex include gel filtration chromatography, ultrafiltration, electrophoresis, ion-exchange chromatography, and immunoaffinity purification with antibodies specific for particular epitopes of the UNC93B or UNC93B-dependent TLR polypeptide or any other binding agent that specifically binds to the polypeptide, optionally attached to a solid support.

Methods of isolating a polypeptide that physically interacts with UNC93B or an UNC93B-dependent TLR are provided. In some embodiments, the method comprises: (a) isolating a complex comprising UNC93B and an UNC93B-dependent TLR from cells; and (b) determining whether the complex contains an additional polypeptide. Optionally, the method includes a step of (c) identifying the additional polypeptide. Methods to perform step (b) include, e.g., mass spectrometry, column chromatography, gel electrophoresis, etc. In certain embodiment the cells are treated with a ligand or stimulus for the UNC93B-dependent TLR. Methods to perform step (c) include, e.g., mass spectrometry, sequencing the polypeptide, etc.

Modified versions of the PCA assays described above are provided, wherein the modification allows detection of an additional polypeptide that interacts with a complex comprising UNC93B or an UNC93B-dependent TLR. The additional polypeptide may interact with either the UNC93B or the UNC93B-dependent TLR or with a surface or binding site that comprises portions of both the UNC93B and the UNC93B-dependent TLR. The modified PCA assays make use of reporter proteins that may be divided into at least 3 fragments. Two of the fragments are fused to UNC93B and UNC93B-dependent TLR polypeptides as described above. A library (e.g., a collection of plasmids) is provided in which each library member encodes a third fragment of the reporter protein fused to a cDNA encoding a polypeptide. Collectively the library includes members representing numerous polypeptides, e.g., hundreds or thousands of different polypeptides that are naturally expressed by cells of interest, e.g., cells that naturally express UNC93B and an UNC93B-dependent TLR. The library is introduced into cells that express (i) a first polypeptide comprising an UNC93B polypeptide and a first fragment of the reporter protein; and (ii) a second polypeptide comprising an UNC93B-dependent TLR polypeptide and a second fragment of the reporter protein. Optionally, cells that have taken up a member of the library are selected. Cells that display reporter protein activity are identified, and the library member contained in the cells isolated and identified, thereby identifying a polypeptide that physically interacts with the complex comprising UNC93B and an UNC93B-dependent TLR. Also provided are a variety of three-hybrid assays, such as that described in U.S. Pub. No. 20040043388, wherein the three-hybrid assay system is appropriately configured to detect proteins or candidate agents that physically interact with complex comprising UNC93B and an UNC93B-dependent TLR.

### V. Methods of Monitoring Immune System Status

A variety of methods that are based at least in part on monitoring the interaction between UNC93B and an UNC93B-dependent TLR are provided. The invention encompasses any method of testing, diagnosis, therapy, or research that involves or includes a step of monitoring such interaction(s).

Methods of monitoring the effect of an agent on immune system cells are provided. In some embodiments, the methods comprise: (a) providing immune system cells; and (b) detecting a complex comprising UNC93B and an UNC93B-dependent TLR in the cells or in a sample obtained from the cells. “Sample obtained from the cells” indicates that the sample may have been subjected to one or more processing steps prior to or as part of the detection step. For example, cells may have been lysed; the lysate may have been fractionated and/or contacted with an antibody, etc. The immune system cells could be obtained from a subject and, optionally, maintained in vitro for days, weeks, months, etc., prior to detection. “Detecting a complex” as used herein refers not only to determining that a complex is present and, optionally, measuring its amount, but also to determining that the complex is not detectable using the detection method used. The immune system cells could include any one or more of the cell types of the immune system. The cells could be from a cell line. In certain embodiments, the cells are contacted with an agent prior to the detection step or are obtained from a subject to which an agent has been administered. The agent could be a candidate agent being evaluated for its ability to modulate TLR signaling and/or to modulate the physical interaction between UNC93B and an UNC93B-dependent TLR. The agent could be an infectious agent or portion thereof, an antigen (e.g., a suspected allergen, a ‘self’ antigen), a known TLR ligand, an agent suspected of being a TLR ligand, a compound known to modulate the immune system (e.g., an immunosuppressive or immunostimulating agent), etc. Exemplary known immunosuppressive agents include glucocorticoids, rapamycin and analogs thereof, FK506 and analogs thereof, and cyclosporin A and analogs thereof. “Known” refers here to the fact that an agent is accepted by those of skill in the art as having a particular property. Thus methods of characterizing an agent comprising: (a) contacting an assay composition comprising an UNC93B polypeptide and an UNC93B-dependent TLR polypeptide with the agent; (b) detecting a complex comprising the UNC93B polypeptide and the UNC93B-dependent TLR polypeptide; and (c) comparing the amount of the complex with a reference value, wherein a difference between the amount of the complex and the reference value is indicative that the agent modulates complex formation. The reference value could be, e.g., the amount of complex formed in the absence of the agent.

Methods of evaluating the immune system of a subject are provided. In some embodiments, the method comprises: (a) providing a sample obtained from the subject, wherein the sample comprises immune system cells; and (b) detecting the presence of a complex comprising UNC93B and an UNC93B-dependent TLR in the sample. The sample could be, e.g., a blood sample, a fluid or tissue biopsy sample (e.g., a lymph node biopsy), etc. The sample could be processed, e.g., as described above. The amount of complex present may be compared against a suitable reference value. The reference value may be obtained from a suitable reference sample. The reference value may be, e.g., (i) the amount of complex that would exist in a similar healthy subject (referred to as a “normal” or “control” value); (ii) the amount of complex that existed in a similar sample obtained from the subject at an earlier time point, e.g., a time point prior to administration of a candidate agent; (iii) the amount of the complex that would be expected to exist in a sample obtained from a subject suffering from an immune system disorder, an infection, an autoimmune disease, etc. A similar subject may be one that is matched in terms of factors such as but not limited to age, sex, general health status, environmental exposures such as smoking, family history, etc., which are commonly used in the art to match control and test subjects for purposes of clinical studies. The reference value may have been previously obtained or the measurement may be made contemporaneously. The reference value may be obtained using the same or a different detection method. In one embodiment, a difference in the amount of the complex relative to the reference value indicates that the subject is at increased risk of (relative to an individual having a “normal value”) or suffers from an immune system disorder or infection. In one embodiment, the result is used to assess the efficacy of a therapeutic agent that has been administered to the subject, wherein the therapeutic agent is an immunosuppressive agent or an immunostimulating agent. In one embodiment, a reduced level of the complex relative to a normal value and/or to a value obtained from a sample obtained prior to administration of the agent indicates that an immunosuppressive agent is efficacious. In one embodiment, an increased level of the complex relative to a normal value and/or to a value obtained from a sample obtained prior to administration of the agent indicates that an immunostimuluating agent is efficacious.

In one embodiment, the method is used to identify a feature of a disorder characterized by aberrant or abnormal function of the immune system. The method comprises: (a) providing a sample obtained from a subject suffering from a disorder characterized by aberrant or abnormal function of the immune system, wherein the sample comprises immune system cells; and (b) detecting a complex comprising UNC93B and an UNC93B-dependent TLR in the sample, wherein detecting an altered amount of the complex relative to a reference value indicates that altered complex formation between UNC93B and an UNC93B-dependent TLR is a feature of the disorder. Altered complex formation between UNC93B and an UNC93B-dependent TLR could be a cause of or contributing factor to the disorder. The disorder could be, e.g., (i) an autoimmune disease, (ii) increased susceptibility to infection relative to most individuals of similar age and general health, (iii) a chronic inflammatory disease, etc. The amount of complex present may be compared against a suitable reference value, which may be obtained from a suitable reference sample. The reference value may be, e.g., the amount of complex that would exist in a similar healthy subject (referred to as a “normal” or “control” value) not suffering from the disorder. A significant difference between the amount of complex present in the sample obtained from the subject as compared with the reference sample indicates, in certain embodiments, that altered formation or stability of the complex plays a contributory or causative role in the disorder. The method may be practiced using samples obtained from a sufficient number of subjects to provide statistically significant results. One of skill in the art will be aware of appropriate statistical tests to perform to establish that a statistically significant correlation exists between the amount of the complex and the existence of the disorder. In one embodiment statistical significance refers to an association that has less than a 5% chance of occurring by chance. The method is thus of use to identify the underlying causes of disorders whose etiology is presently poorly understood and, once the correlation is established, can be used to diagnose the disorder and/or monitor its progression or response to therapy. Furthermore, such a result would suggest, in certain embodiments, that administration of agents that modulate activity of an UNC93B-dependent TLR (including, but not limited to, agents that modulate the interaction between UNC93B and an UNC93B-dependent TLR) would be suitable therapies for the subject. The invention thus provides a method of selecting a suitable therapeutic agent for a subject suffering from a disorder characterized by aberrant or abnormal function of the immune system.

### VI. Additional Aspects

A computer-readable medium that stores information obtained from performing any of the screening methods of the invention is also provided. The information can be expressed in any convenient format. Typically the information will be stored in a database. The information may, for example, identify compounds by name, structure, identification number, or any other suitable means. The information may identify one or more compounds that modulate the interaction of UNC93B and an UNC93B-dependent TLR and/or that modulate activity of one or more TLRs by a mechanism that involves modulating the interaction between UNC93B and an UNC93B-dependent TLR. The information can be qualitative or quantitative. Such databases are an aspect of the invention. An additional aspect of the invention is a method that comprises receiving or sending, in electronic or tangible format, results from an inventive screen.

Systems for performing high throughput and/or high content screening that include apparatus for performing the methods, e.g., robots, plate readers, software, and/or computers as described above are provided.

A variety of kits are provided. The kits may, for example, contain (i) a first nucleic acid construct that encodes a recombinant polypeptide comprising an UNC93B-dependent TLR polypeptide and a first fragment of a reporter protein or RET protein; and (ii) a second nucleic acid construct that encodes a recombinant polypeptide comprising an UNC93B polypeptide and a second fragment of a reporter protein or a RET protein. The kit may further contain (i) a substrate for the reporter protein or RET protein; (ii) cells for expressing the polypeptides; (iii) instructions for use, etc. Kits may contain one or more ER or ELP markers (e.g., dyes that accumulate in the ER or ELP), or one or more nucleic acid constructs that encodes a detectably labeled ER or ELP polypeptide marker. Kits may contain cells that express any one or more of the recombinat polypeptides of use in the assays. Kits may include one or more vessels or containers so that certain of the individual reagents may be separately housed. The kits may also include a means for enclosing the individual containers in relatively close confinement for commercial sale, e.g., a plastic, cardboard, or styrofoam box, in which instructions are enclosed.

The invention also provides a method of targeting an UNC93B-dependent TLR to a desired cellular location, e.g., a location other than the ER or ELP. The method comprises expressing the TLR in a cell that expresses an UNC93B polypeptide that contains a domain that targets the UNC93B polypeptide to the desired cellular location. The TLR associates with the modified UNC93B polypeptide and is translocated to the cellular domain. For example, the UNC93B polypeptide may contain a domain that targets it to the plasma membrane, to the mitochondria, etc. In some embodiments, a plasma membrane sorting sequence such as that derived from the yeast membrane protein Ist2p (Gene ID 852382 (Saccharomyces cerevisiae) is used. One of skill in the art will be aware of suitable sorting sequences. It will be appreciated that the UNC93B and TLR polypeptides may also contain domains that initially target them to the ER, wherein they may associate. The TLR polypeptide may be a chimeric polypeptide containing portions of two or more different TLRs. The methods are of use, e.g., to modify responsiveness of a cell to TLR ligands, etc.

### VII. Uses for Agents that Modulate the Physical Interaction Between Unc93B and UNC93B-Dependent TLR(s)

Agents that modulate the physical interaction between an UNC93B-dependent TLR and UNC93B have a variety of different uses. In accordance with certain embodiments of the invention, agents identified using the inventive methods are useful as immunomodulators. The term “immunomodulator” refers to an agent capable of modulating one or more functions or activities of the immune system. Immunomodulators include immunosuppressants and immunostimulating agents.

In some embodiments, an immunomodulator enhances or inhibits a function or activity of one or more types or categories of immune system cells, e.g., T cells, B cells, or subset(s) thereof. Subsets of immune system cells are typically characterized and/or defined by the expression of particular cell surface markers or combinations thereof and/or based on their production of particular cytokines. Effector cells include a variety of immune system cells that play a role in the body's defense against foreign “non-self” antigens or, in the case of autoimmune disease, inappropriately attack self antigens (autoantigens). Effector cells of various types may engage in a variety of effector activities including cytokine synthesis and release, synthesis and release of other inflammatory mediators, cell killing, phagocytosis, etc. In some embodiments, the immune system cells are effector cells. In some embodiments, the immune system cells are helper T cells. Helper T cells are T lymphocytes that belong to the CD4+ subset. They have been divided into at least two kinds: Th1 cells participate in cell-mediated immunity and contribute to the control of such intracellular pathogens as viruses and certain bacteria, e.g., Listeria and Mycobacterium tuberculosis. Th2 cells provide help for B cells and, in so doing, contribute to antibody-mediated immunity. Th2 cells are characterized by their secretion of IL-4, IL-5, IL-9 and IL-13, while Th1 cells characteristically secrete IL-2 and interferon-γ. A “Th1-mediated disease” is one whose induction and/or progression is caused at least in part by Th1 cells and/or cytokines produced thereby. Rheumatoid arthritis is an exemplary Th1-mediated disease. A “Th2-mediated disease” is one whose induction and/or progression is caused at least in part by Th2 cells and/or cytokines produced thereby. Exemplary Th2-mediated diseases include allergic diseases (also called atopic diseases) such asthma. In certain embodiments of the invention an immunomodulator shifts the immune response away from a Th2-mediated response and towards a Th1-mediated response. In other embodiments, an immunomodulator shifts the immune response away from a Th1-mediated response and towards a Th2-mediated response. In other embodiments, the immunomodulator reduces the magnitude of a Th1-mediated response, a Th2-mediated response, or both.

In some embodiments, the immune systems cells are antigen presenting cells. In some embodiments, the immune system cells are dendritic cells. Dendritic cells (DC) include professional antigen-presenting cells that capture antigens in the peripheral tissues and migrate via lymph or blood to the T cell area of draining lymph nodes and spleen where they present processed antigens to naive T cells, initiating antigen-specific primary T cell responses. Methods of the invention and/or agents identified according to the invention can be used to modulate, e.g., inhibit, the interaction(s) between dendritic cells and T cells. Such interactions include the adhesion of T-cells to dendritic cells, for instance in dendritic cell-T cell clustering, T-cell activation and further include all interactions that rely on direct cell-to-cell contact or close proximity of dendritic cells and T cells, such as processes involved in generating an immune response, in particular during the initial stages of such a response, e.g., sensitization/activation of T-lymphocytes, (i.e. presentation of antigen and/or MHC-bound peptides to T-cells) and co-stimulation of T cells; as well as processes such as cytokine signaling, endocytosis and transepithelial transport. The methods and agents can therefore be used to influence the immunomodulatory ability of dendritic cells; to modulate, and in particular reduce, dendritic cell-mediated (primary) T cell responses. The methods and agents may also modulate the activation of other receptors on T cells which are dependent upon the adhesion or close proximity of dendritic cells to T cells.

In some embodiments, the immune system cells are B cells, e.g., antibody producing cells such as plasma cells or precursors thereof.

In some embodiments, the immune system cells are regulatory T cells (T_{reg} cells).

In some embodiments, the immune system cells are macrophages.

In some embodiments, an inhibitor of the interaction between UNC93B and one or more TLRs inhibits production of one or more pro-inflammatory cytokines by a mammalian cell. Mammalian cells known to produce pro-inflammatory cytokines include monocytes, macrophages, neutrophils, epithelial cells, osteoblasts, fibroblasts, smooth muscle cells, and neurons. The mammalian cell may be maintained in culture or may be in a living subject, e.g., an individual suffering from or at increased risk of a condition mediated by or resulting at least in part from pro-inflammatory cytokine release. The inhibitor may be administered to treat the individual. In some embodiments, the agent is contacted with cells ex vivo, i.e., outside the body. The cells may then be administered to a subject. Research purposes are also within the scope of the invention since the agents are useful to further characterize TLRs and their signalling pathways.

A number of deleterious conditions are characterized by systemic or local responses mediated at least in part by production and release of pro-inflammatory cytokines. In some instances excessive inflammation, cytokine release, and/or activation of immune system cells can do significant damage to body tissues and organs. For example, accumulation of fluids and immune system cells in the lungs can block the airways, potentially leading to death. The invention relates in various embodiments to the inhibition of acute inflammatory responses, which in some embodiments are caused at least in part by release of pro-inflammatory cytokines. The invention further relates to the inhibition of chronic inflammatory responses.

Conditions characterized by inflammation and/or excessive pro-inflammatory cytokine release include peritonitis, pancreatitis, ulcerative colitis, Crohn's disease, acute colitis, ischemic colitis, diverticulitis, epiglottitis, cholangitis, cholecystitits, hepatitis, enteritis, Whipple's disease, asthma, allergy, anaphylactic shock, organ ischemia, reperfusion injury, hay fever, sepsis, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitits, alveolitis, bronchiolitis, pharyngitis, pleurisy, sinusitis, burns, dermatitis, dermatomyositis, sunburn, urticaria, vasculitis, angiitis, endocarditis, arteritis, atherosclerosis, thrombophlebitis, pericarditis, myocarditis, myocardial ischemia, rheumatic fever, asthma, allergy, hypersensitivity reaction, Alzheimer's disease, coeliac disease, congestive heart failure, adult respiratory distress syndrome, meningitis, encephalitis cerebral infarction, stroke, Guillane-Barre syndrome, neuritis, neuralgia, spinal cord injury, paralysis, uveitis, arthritides, arthralgias, osteomyelitis, fasciitis, Paget's disease, gout, periodontal disease, and synovitis. See, e.g., U.S. Pat. No. 6,838,471.

Release of pro-inflammatory cytokines can have pathogenic consequences in a number of infectious diseases caused by viruses, bacteria, fungi, or parasites. Viral diseases of interest include those caused by any DNA or RNA virus that is pathogenic in a mammalian host. Exemplary viruses include those falling within the following groups: Arbovirus, Adenoviridae, Arenaviridae, Arterivirus, Birnaviridae, Bunyaviridae, Caliciviridae, Circoviridae, Coronaviridae, Flaviviridae, Hepadnaviridae, Herpesviridae, Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae, Reoviridae, Retroviridae, and Togaviridae. In certain embodiments the disease is caused by influenza virus, parainfluenza virus, respiratory syncytial virus, metapneumovirus, herpes viruses, human immunodeficiency virus, hepatitis B virus, hepatitis C virus, or cytomegalovirus. Diseases caused by gram-positive or gram-negative bacteria, mycobacteria, fungi such as Candida or Aspergillus, helminths, etc., are also of interest. Exemplary bacteria and fungi include those falling within the following groups Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionella, Leptospires Listeria, Mycoplasmatales, Neisseriaceae (e.g., Acinetobacter, Menigococci), Pasteurellacea (e.g., Actinobacillus, Heamophilus, Pasteurella), Pseudomonas, Rickettsiaceae, Chlamydiaceae, Treponema, and Staphylococci. Of particular interest are diseases caused by infectious agents that express PAMPs recognized by one or more UNC93-dependent TLRs. Parasitic disease of interest include babesiosis, malaria, cryptosporidiosis coccidiomycosis, histoplasmosis, toxoplasmosis, trypanosomiasis, diseases caused helminths, etc. In some embodiments an agent that inhibits interaction between UNC93B and an UNC93B-dependent TLR is administered to inhibit or reduce pro-inflammatory cytokine release associated with an infection.

In some embodiments, the immunomodulator suppresses or inhibits one or more aberrant or undesired responses of the immune system against an autoantigen. The autoantigen may be, e.g., single- or double-stranded DNA or RNA. Such immunomodulators are useful for treatment of a variety of autoimmune disorders. Exemplary autoimmune disorders include type I diabetes (e.g., juvenile onset diabetes), multiple sclerosis, scleroderma, ankylosing spondylitis, sarcoid, pemphigus vulgaris, myasthenia gravis, systemic lupus erythemotasus, rheumatoid arthritis, juvenile arthritis, Behcet's syndrome, Reiter's disease, Berger's disease, dermatomyositis, Wegener's granulomatosis, autoimmune myocarditis, anti-glomerular basement membrane disease (including Goodpasture's syndrome), dilated cardiomyopathy, thyroiditis (e.g., Hashimoto's thyroiditis, Graves' disease), and Guillane-Barre syndrome.

In some embodiments, the immunomodulator is used to treat asthma, allergy, or hypersensitivity reaction.

In some embodiments, the immunomodulator suppresses or inhibits a response against transplanted cells, tissues, or organs. Immunomodulators that suppress or inhibit an immune response against non-self (e.g., allogeneic or xenogeneic) antigens are of use to reduce the likelihood of rejection of cell, tissue, or organ transplants, tissue grafts, etc. Immunomodulators that suppress or inhibit an immune response are also of use to treat graft-versus-host disease.

Chronic inflammation is an important factor contributing to development of a number of diseases that are not typically considered to be classical “autoimmune” diseases. Chronic inflammation has been implicated in tumorigenesis and tumor progression, atherosclerosis, Alzheimer's disease, heart failure, and a number of other diseases. Agents that inhibit the interaction between UNC93B and an UNC93B dependent TLR are of use to inhibit development and/or progression of such diseases. Tumors include solid tumors (e.g., cancer, sarcoma) and hematologic malignancies. Cancers include but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g., small cell and non-small cell); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. Some cancer cells are metastatic cancer cells.

In some embodiments, the immunomodulator enhances a response of the immune system against an undesired foreign antigen or tumor antigen. Such immunomodulators may be useful, e.g., to reduce the likelihood of tumor escape from immunosurveillance. The inventive therapeutic strategy is of use, in some embodiments, to prevent emergence of metastases, e.g., by increasing immune surveillance.

Candidate agents identified according to the inventive methods can be tested in animal models to further explore their effects on immune system function and/or further evaluate therapeutic potential. Animal models for a variety of autoimmune, chronic inflammatory diseases, and infectious diseases are known.

An agent identified according to the invention is delivered in an effective amount, by which is meant an amount sufficient to achieve a biological response of interest. In certain embodiments the agent is administered to a mammalian subject suffering from or at increased risk of a condition mentioned herein in a therapeutically effective amount, e.g., an amount sufficient to ameliorate at least one symptom of the disease to a clinically meaningful extent. An agent can be administered for therapeutic purposes after onset of symptoms or prior to onset (prophylactically). For example, an agent may be administered after exposure to an infectious agent or allergen but before symptoms have developed. An individual may be at risk if he or she has a family history of a condition, falls into a recognized risk category, has been exposed to an infectious agent, has received or will receive a transplant, etc.

When administered to an individual, agents identified according to the invention can be administered as a pharmaceutical composition comprising a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters.

A pharmaceutically acceptable carrier can contain physiologically acceptable compounds that act, for example, to stabilize or to increase the absorption of the active therapeutic compound. The physiologically acceptable compounds include, for example, carbohydrates, such as glucose, sucrose or dextrans, antioxidants, such as ascorbic acid or glutathione, chelating agents, buffers, low molecular weight proteins or other stabilizers or excipients. One skilled in the art would know that the choice of a pharmaceutically acceptable carrier, including a physiologically acceptable compound, depends, for example, on the route of administration of the composition. The pharmaceutical composition could be in the form of a liquid, gel, lotion, tablet, capsule, ointment, transdermal patch, etc. In some embodiments, the pharmaceutical composition contains a vaccine antigen, which term is used to refer to any antigenic substance that is administered to a subject in order to provide or enhance immunity towards a specific infectious agent or against a tumor.

One skilled in the art would know that a pharmaceutical composition can be administered to a subject by various routes including, for example, oral administration; intramuscular administration; intravenous administration; anal administration; vaginal administration; parenteral administration; nasal administration; intraperitoneal administration; subcutaneous administration and topical administration. One skilled in the art would select an effective dose and administration regimen taking into consideration factors such as the patient's weight and general health, the particular condition being treated, etc.

The pharmaceutical composition also can be delivered by means of a microparticle or nanoparticle or a liposome or other delivery vehicle or matrix. A number of biocompatible polymeric materials are known in the art to be of use for drug delivery purposes. Examples include polylactide-co-glycolide, polycaprolactone, polyanhydride, and copolymers or blends thereof. Liposomes, for example, which consist of phospholipids or other lipids, are nontoxic, physiologically acceptable and metabolizable carriers that are relatively simple to make and administer.

The agents could be administered in combination with other agents useful for the same disease, either in the same composition or individually. Such agents could be, e.g., immunosuppressive agents, immunostimulating agents, anti-infective agents (including anti-bacterial, anti-viral, anti-fungal, and anti-parasite agents), etc.

### VIII. Insight into Role of UNC93B in TLR Signaling

As noted above, Toll-like receptors are involved in the perception and processing of signals delivered by viral and microbial products and coordinate both innate and adaptive immunity. Mutations in UNC93B cause signaling defects for multiple TLRs and prompted us to explore the mechanism of its involvement in TLR signaling. By expression of epitope-tagged UNC93B and using newly raised polyclonal antibodies that recognize endogenous UNC93B, UNC93B was characterized as a glycosylated ER-resident protein. The mutant UNC93B (H412R) protein exhibits distinct migration patterns in SDS-PAGE compared to wild type UNC93B. Its behavior suggests a role for His412 in the intramolecular organization of UNC93B, as mild denaturation with SDS is likely to preserve at least some features of secondary structure. Nonetheless, the 3d mutation (H412R) in UNC93B does not compromise biosynthesis and maturation of the mutant protein, and both wild type and mutant proteins are equally stable.

As discussed above, to study the role of UNC93B in TLR signaling, UNC93B binding proteins were identified using a large-scale preparative immunoprecipitation in conjunction with mass spectrometry. Wild type but not mutant UNC93B was found to interact with TLRs 3, 7, 9 and 13. TLR4 did not interact with either wild type or mutant UNC93B. The number of peptides and the extent of sequence coverage of TLRs identified by mass spectrometry suggest that these TLRs are well-represented among proteins that bind to UNC93B. The interaction between wild type UNC93B and TLRs (TLRs 3, 7 and 9) was confirmed by additional biochemical analyses in cell lines and primary cells. The interactions between wild type UNC93B and TLRs (TLRs 3, 7, 9 and 13) and the absence of interaction between UNC93B and TLR4 correlate well with the phenotype of the 3d mice, which show specific defects in signaling via TLRs 3, 7, and 9, but not TLR4 (Tabeta et al., 2006). In addition, expression of TLR13 in a macrophage cell line was demonstrated. TLR13 was identified by the homology search of TIR domain-containing proteins in mouse ESTs, but no function has been assigned to it. Without wishing to be bound by any theory, signaling via this less well characterized TLR is likely to be affected in 3d mice.

As demonstrated herein, ER-localized UNC93B physically interacts with TLR9. UNC93B retains full Endo H sensitivity. By using chimeric TLRs, it was shown that the transmembrane domains of TLR3 and 9 are responsible for binding to UNC93B. When the transmembrane domain of TLR4 is substituted for the transmembrane domain of either TLR3 or 9, the chimeric TLR4 proteins acquire the ability to interact with UNC93B. In contrast, TLR3 and TLR9 chimeras equipped with the TLR4 transmembrane domain no longer bind to UNC93B.

The strength of interaction between UNC93B with its client TLRs is robust, as judged from the quantities of TLRs detected by direct immunoprecipitation and that found in association with UNC93B. This result suggests that a considerable fraction, if not the majority of TLRs 3, 7 and 9, may be associated with UNC93B. The results of pulse chase analyses are likewise consistent with stable association of UNC93B with TLRs. These observations raise the question as to the function of UNC93B and how it participates in TLR signaling. One possibility is that UNC93B acts as an ER chaperone for TLRs 3, 7, 9 and 13. However, without wishing to be bound by any theory, given the fact that the transmembrane segment of a TLR is sufficient to dictate interactions with UNC93B and that interaction between UNC93B and TLRs is not transient but is maintained for a prolonged period of time, without wishing to be bound by theory, a classical chaperone function for UNC93B is less likely. Chaperone-client interactions are usually transient, and for glycoproteins involve mostly the luminal/extracellular domains. Moreover, the expression level of TLR7 is not altered in BM-DCs from 3d mice compared to wild type mice, arguing against the possibility that UNC93B would act by stabilization of its client TLRs. Instead, by analogy with the function of another ER resident protein gp96, which is essential for TLR4/MD-2 complex assembly (Randow and Seed, 2001), UNC93B may be involved in the assembly of UNC93B-dependent TLRs with so-far unidentified proteins that are essential for either ligand recognition or signal transduction. Alternatively, UNC93B may play a role in retaining the client TLRs in the ER until they are ready to traffic to endosomes. TLR9 resides in the ER until activated (Latz et al., 2004; Leifer et al., 2004). It is likely that a large proportion of TLR3 colocalizes with TLR9 at steady state (Kajita et al., 2006; Nishiya et al., 2005).

MD-2 directly interacts with TLR4 and plays an important role in the recognition of LPS (Shimazu et al., 1999). In addition, MD-2 was suggested to contribute to the surface localization of TLR4 (Nagai et al., 2002). In MD-2 deficient mouse embryonic fibroblasts, TLR4 is retained in the Golgi apparatus. While MD-2-bound TLR4 is directed to the cell surface constitutively, UNC93B appears to play a role in the transport of its client TLRs to endosomes in a stimulation-dependent manner. Trafficking of TLR9 from ER to lysosomal compartments after uptake of CpG in dendritic cells therefore, depends on the interaction between UNC93B and TLR9, based on available evidence (see Examples 9-14). Since both UNC93B and TLRs retain full Endo H sensitivity even after TLR stimulation, they may travel to endosomes via an unconventional anterograde route that does not involve the Golgi apparatus, should the UNC93B/TLR complex indeed traffic together to endosomes as appears to be the case (see Examples 9-14). Tabeta, et al. reported no discernible changes in TLR localization in cells from 3d mice (Tabeta et al., 2006). High resolution microscopy studies on localization of TLRs after proper stimulation, as described in Examples 9-14, provided new insights into the role of UNC93B in TLR trafficking and provide methods to identify agents that modulate the UNC93B/TLR interaction and/or modulate TLR signaling.

Upon activation, many receptors recruit adapter molecules onto which various downstream signaling molecules assemble for efficient and coordinated signal transduction. For some receptors a scaffolding protein that constitutively binds the receptor provides a platform to which signaling molecules are recruited. UNC93B may serve as such a scaffold for TLR signaling or be an integral part of a signaling unit. The exposed loops and N-/C-termini of UNC93B could serve to organize and orient the other components of the TLR signaling complex. The stably maintained interactions between UNC93B and TLRs, even after activation of TLRs, support this hypothesis. Initial large scale immunoprecipitation of UNC93B and the subsequent mass spectrometry analysis did not identify any prominent signaling molecules of the TLR signaling pathways. The experimental approach described here provides a method to identify additional proteins that bind to UNC93B after stimulating cells with TLR agonists.

The TLR signaling defects in cells from 3d mice are mimicked by pretreating wild type cells with chloroquine or bafilomycin, agents that inhibit endosome acidification, but the 3d mutation does not affect the pH of different intracellular organelles (Tabeta et al., 2006). Therefore, without wishing to be bound by any theory, UNC93B does not seem to be directly involved in endosome acidification. Human UNC93B1 protein contains a region of weak homology to the bacterial ABC-2 type transporters (Kashuba et al., 2002). In addition, homology searches of the conserved domains (NCBI blast, rpsblast) identified a second domain (residues 101 and 185 of UNC93B) with similarity to a bacterial transporter (MelB, E-value of 0.03). Even though sequence homology is weak in both cases, these observations raise the possibility that UNC93B could have a yet unidentified transporter function. The ectodomain of the microbial nucleotide-sensing TLRs (TLRs 3, 7, and 9) faces the lumen of the ER and endosomes. It is likely that viral DNAs and RNAs need to gain access to such an environment for signaling through their cognate TLRs. Therefore, UNC93B may participate in such processes and position the TLRs for efficient recognition of their agonists.

The exact identities of intracellular compartments where the nucleotide-sensing TLRs receive stimulating signals from microbial DNAs and RNAs are still an open question. Studies using chemical inhibitors that prevent endosome acidification have proposed the endosomes as the location where UNC93B-dependent TLR9 initiates signaling and potentially recognizes ligands (Ahmad-Nejad et al., 2002; Barton et al., 2006). However, upon addition of CpG DNA, even in MyD88 deficient dendritic cells, TLR9 travels normally from the ER to endosomes where internalized CpG accumulates (Latz et al., 2004). If translocation of TLR9 to endosomes is indeed a signal-mediated event, as suggested by the results in Examples 9-14, these data imply that either i) TLR9 receives a stimulatory signal of CpG DNA before reaching the endosomes—perhaps in the ER, or ii) there is an additional molecule that senses CpG and triggers translocation of TLR9 in a MyD88-independent manner. The possibility that TLR9 may sense CpG in the ER or ER-derived structures deserves consideration. Pathways for retrograde transport of microbial products to the ER include delivery of the intact pathogen itself, as exemplified by SV40 and polyoma virus (Spooner et al., 2006). Bacterial toxins such as cholera toxin and shiga toxin likewise travel from the cell surface to the ER, from which they are discharged into the cytoplasm to intoxicate the cells exposed to the toxin (Spooner et al., 2006). In addition, it has been alleged that exogenously added soluble proteins can access the ER lumen in dendritic cells (Ackerman et al., 2005). Therefore, the delivery of TLR ligands to the ER itself is certainly a possibility. Potentially, UNC93B could play a role in the perception of ER-delivered microbial nucleotides in concert with the UNC93B-dependent TLRs.

The reported phenotype of 3d mice includes defects in cross presentation and MHC Class II-mediated antigen presentation. Despite the defects in presentation of exogenous antigens, the subcellular distribution of MHC Class I and II proteins was not affected (Tabeta et al., 2006). Consistent with this observation, biosynthesis, maturation and assembly of MHC Class I and II molecules were identical in BM-DCs from wild type and 3d mice. While it remains unclear how UNC93B participates in antigen presentation, it is worth noting that a series of recent, yet controversial observations suggests the involvement of the ER or ER-derived intracellular compartments in crosspresentation of exogenous antigens (Ackerman et al., 2006; Ackerman et al., 2005; Imai et al., 2005). The role of UNC93B in crosspresentation deserves to be further explored in this context.

In summary, it has been demonstrated that wild type but not mutant UNC93B (H412R) physically interacts with TLRs 3, 7, 9 and 13. It has also been demonstrated that UNC93B delivers nucleotide-sensing TLRs to the endosomal/lysomal/phagosomal compartment. The established interaction between UNC93B and TLRs and role of UNC93B in TLR trafficking sheds new lights on the 3d mutation and its TLR signaling-defective phenotype. The prominent ER localization of UNC93B and of the TLRs to which it binds raises the intriguing possibility that the ER itself may serve as a compartment from which TLR signaling is initiated.

### EXAMPLES

### Materials and Methods for Examples 1-8

Cell lines. Murine RAW 264.7 macrophages (ATCC TIB-71) and human embryonic kidney (HEK) cells 293-T (ATCC CRL-11268) were maintained in DMEM containing 10% heat-inactivated fetal calf serum (IFS) and penicillin/streptomycin (P/S). Murine A20 B cells (ATCC TIB-208) were maintained in RPMI1640 medium supplemented with 10% IFS and P/S. 293-T cells were transfected with FuGene-6 (Roche) according to the manufacturer's instructions.

Animals. C57BL/6 wild type mice were purchased from Taconic. The TLR7^{-/-} (Hemmi et al., 2002) and TLR9^{-/-} (Hemmi et al., 2000) mice were obtained from Dr. Ann Marshak-Rothstein (Boston University). All animals were maintained under specific pathogen-free conditions and experiments were performed in accordance with institutional, state, and federal guidelines.

Antibodies and reagents. Antibodies against mouse UNC93B were generated against the N-terminal (anti-UNC-N, aa 1-59) and the C-terminal (anti-UNC-C, aa 515-598) region. Three peptides were chosen for each region with the antigen profiler from open biosystems or the world wide web at (openbiosystems.com) and synthesized with a cysteine residue added to the N-terminus by the MIT Center for Cancer Research Biopolymers Laboratory. N-terminal UNC93B peptides: (1N) C-DRHGVPDGPEAPLDE, (2N) C-PDGPEAPLDELVGAY, (3N) C-GAYPNYNEEEEERRYYRRK. C-terminal UNC93B peptides: (1C) C-LQQGLVPRQPRIPKP, (2C) C-RYLEEDNSDESDMEG, (3C) CPYEQALGGDGPEEQ. Peptides were analyzed by HPLC and mass spectrometry for purity and then coupled individually to keyhole-limpet haemocyanin (KLH, Pierce) via the cysteine residue using sulfosuccinimidyl 4-[N-maleimidomethyl]-cyclohexane-1-carboxylate (sulfo-SMCC, Pierce) according to the manufacturer's recommendations. After coupling, the three peptides (1N, 2N, 3N or 1C, 2C, 3C) were mixed and antisera were raised in rabbits by an outside vendor (Covance). The anti-UNC-C polyclonal serum was affinity purified using the three peptides of the C-terminal region (1C, 2C and 3C) that are coupled to the resin using the Sulfo Link Kit (Pierce). Affinity-purified anti-UNC-C antibodies were used for immunoprecipitations, immunofluorescence and immunoblotting. The rabbit polyclonal TLR7 antibody was purchased from Imgenex. The Flag antibody (M2, mouse monoclonal) was from Sigma. The anti-HA affinity matrix (3F10, rat monoclonal) was purchased from Roche. The anti-HA 12CA5 antibody (mouse monoclonal) was produced in our laboratory from hybridoma cells. The anti-myc (9E10, mouse monoclonal) antibody was purchased from Invitrogen. The MHC class II antibody is a hamster monoclonal antibody (clone N22). Imiquimod (R837) and gardiquimod were purchased from Invivogen, CpG DNA (1826-CpG) from TIB Molbiol, and LPS (E. coli 026:B6) from Sigma.

DNA cloning. Murine UNC93B (BC018388) was C-terminally fused with the Flag tag followed by the Tobacco Etch Virus (TEV) protease cleavage site (ENLYFQG) and the HA tag (UNC93B-HA, WT; see scheme in FIG. 1a). The point mutant UNC93B (H412R) was generated by sequential PCR with primers carrying the point mutation CAC (His) to CGC (Arg) and C-terminally fused with the Flag tag followed by the TEV protease cleavage site and the HA tag (UNC93B-HA, H412R). UNC93B-HA WT and H412R were cloned into the retroviral vector pMSCVneo (Clontech) and stable cell lines in RAW or A20 B cells were established by retroviral transduction and selection with geneticin (see below).

The TLR9 cDNA in pcDNA3.1 was kindly provided by Stefan Bauer (Bauer et al., 2001). C-terminally myc-tagged murine TLR3 (BC099937), TLR4 (BC029856) and TLR9 (AF348140) were generated by PCR and cloned into the pMSCVpuro (Clontech) or pcDNA3.1 vector (Invitrogen). The TLR chimeras were constructed by PCR “sewing” with cDNA corresponding to the following amino acids of the TLRs: TLR4-3-4: 1-625 of TLR4, 698-726 of TLR3 and 660-835 of TLR4; TLR3-4-3: 1-697 of TLR3, 626-659 of TLR4, 727-905 of TLR3; TLR4-9-4: 1-625 of TLR4, 811-839 of TLR9, 660-835 TLR4; TLR9-4-9: 1-810 of TLR9, 626-659 of TLR4, 840-1032 of TLR9. All TLR chimeric constructs were C-terminally myc-tagged and cloned into the pMSCVpuro vector. The sequence of all constructs generated by PCR was verified.

Retroviral transduction. HEK 293-T cells were transfected with plasmids encoding VSV-G or Env, Gag-Pol and pMSCV-UNC93B-HA (WT) or pMSCV-Unc93b-HA (H412R). 24 h post transfection, medium containing viral particles was collected, filtered through a 0.45 μm membrane and added to RAW macrophages (VSV-G) or A20 B cells (Env) cells. Cells were spun for 2 h at 2.000 rpm, medium was changed and cells were selected with geneticin (Invitrogen, 750 μg/ml) two days after transduction.

Preparation of bone marrow-derived dendritic cells. BM-DCs were prepared from C57BL/6, UNC93B mutant (3d), TLR7 deficient (TLR7^{-/-}) or TLR9 deficient (TLR9^{-/-}) mice as described(Maehr et al., 2005).

TNF ELISA assay. BM-DCs derived from wild type (C57BL/6), UNC93B mutant (3d), TLR7 or TLR9 knockout mice were stimulated for 4 h with increasing concentrations of TLR agonists LPS (TLR4), imiquimod (TLR7) or CpG DNA (TLR9). The conditioned medium was collected and analysed by ELISA using the hamster anti-mouse/rat TNF antibody (BD Biosciences) as a capture antibody and a rabbit anti-mouse biotin labeled secondary antibody (BD Biosciences).

Stimulation with TLR agonists. A20 B cells either not transduced or stably transduced with UNC93B-HA WT or H412R were metabolically labeled with ³⁵S-methionine/cysteine for 4 h (pulse). TLR agonists were added to the cells for the final hour of the pulse at the following concentrations: imiquimod: 10 μM, gardiquimod: 1 μM, CpG DNA: 1 μM. Cells were lysed in 1% digitonin lysis buffer and immunoprecipitation was carried out as indicated in the figure legend.

Pulse-chase, immunoprecipitation and Endo H/F assay. In brief, cells were starved for methionine and cysteine in Met/Cys-free DMEM (starvation medium) for 30 min, pulsed for different time periods (see figure legends) with ³⁵S-labeled methionine/cysteine (Perkin Elmer) in starvation medium supplemented with dialyzed IFS, and chased with an excess of non-radioactive amino acids in regular DMEM for various time periods (see figure legends). Cells were lysed in either of the following lysis buffers supplemented with the complete protease inhibitors (Roche) as indicated in the figure legends: RIPA (20 mM Tris-HCl, pH 7.4, 1 mM EDTA, 100 mM NaCl, 1% Triton X-100, 0.5% sodium deoxycholate, 0.1% SDS), or buffer containing 50 mM Tris-HCl, pH 7.4, 150 mM NaCl, 5 mM EDTA with either 1% NP-40 or 1% digitonin as detergent. Lysates were equalized for incorporation of radioactive material with ³⁵S counts in the trichloroacetic acid precipitate and immunoprecipitated with the indicated antibodies. Washes were performed with the same buffers used for lysis except for digitonin lysates/immunoprecipitations which were washed with 0.1-0.2% digitonin-containing buffer. Re-immnuoprecipitations were carried out as follows: Protein-antibody complexes were dissolved by mild denaturation with 1% SDS and 1% β-mercaptoethanol for 1 h at 37° C. Subsequently, SDS and β-mercaptoethanol were diluted to 0.1% by addition of 1% NP40 lysis buffer and re-immunoprecipitations were carried out with the indicated antibodies. Immunoprecipitates were subjected to 10% SDS-PAGE and fluorography. Digestions with Endoglycosidase H and peptide: N-Glycosidase F were performed, where indicated, in accordance with the manufacturer's instructions (New England BioLabs).

Immunoprecipitations and immunoblotting. In brief, cells were lysed in 1% NP40, 1% digitonin or RIPA buffer (see figure legends) and immunoprecipitation was carried out with the antibodies indicated in figure legends. The samples were subjected to 10% SDS-PAGE, transferred to a nitrocellulose membrane and immunoblotted with the antibodies indicated.

Large-scale affinity purification and mass spectrometry. The procedure was adapted from Lilley et al. (Lilley and Ploegh, 2004). In brief, four billion RAW cells stably expressing UNC93B-HA (WT) or UNC93B-HA (H412R) or no exogenous UNC93B protein (control cells) were each lysed in 30 ml of ice-cold lysis buffer (1% digitonin, 50 mM Tris-HCl, pH 7.4, 150 mM NaCl, 5 mM EDTA, complete protease inhibitors (Roche)) with rocking at 4° C. for 1 h. The lysate was cleared of cell debris and nuclei by centrifugation at 20,000 g for 15 min. UNC93B-HA and associated proteins were retrieved from 150 mg of cleared lysate by immunoprecipitation with 330 μl of compact anti-HA antibody beads. After incubation for 3 h, beads were extensively washed in wash buffer (same composition as lysis buffer, except with 0.1% digitonin and without protease inhibitors). Bound material was eluted by incubation with 100 U TEV protease (Invitrogen) at 4° C. overnight in 200 μl wash buffer. The eluate was exchanged into 20 mM NH₄CO₃ (pH 8.0) with 0.1% SDS with the use of Sephadex G-25 resin (GE Healthcare) and concentrated in a Speed Vac. Reducing SDS loading buffer was added to the sample, polypeptides were separated by 10% SDS-PAGE and revealed by silver staining. The bands of interest were excised, subjected to trypsinolysis, separated by liquid chromatography, analysed by MS/MS and database-searched as described (Lilley and Ploegh, 2004).

### Example 1

### Wild Type and Mutant UNC93B Proteins Retain Endo H Sensitivity and Show Similar Stability

The murine Unc93b gene comprises 11 exons and gives rise to a protein of 598 amino acids. Topology prediction programs suggest that UNC93B spans the membrane 12 times and the 3d mutation (H412R) is located within transmembrane domain 9 (FIG. 1a). UNC93B has two putative N-linked glycosylation sites (consensus NxS/T), N²⁵¹HT and N²⁷²KT (FIG. 1a). Polyclonal rabbit antibodies were raised against several peptide sequences of the N-terminal and C-terminal portions of UNC93B. The antibodies showed reactivity with both wild type and mutant UNC93B, as assessed by immunoblotting and immunoprecipitation on cell extracts prepared from a variety of sources (data not shown).

Polar residues in transmembrane domains are involved in helix-helix interactions within a multi-transmembrane domain-containing protein to aid helix packing or participate in protein-protein interactions with a neighboring membrane protein (Curran and Engelman, 2003). Missense mutations involving the loss or gain of an arginine residue in a predicted transmembrane domain are often associated with protein misfolding and malfunction, as found in many human diseases (Partridge et al., 2004). Therefore, the effects of the H412R mutation on expression, maturation and stability of the UNC93B protein were assessed. An epitope-tagged version of UNC93B in which the Flag-TEV-HA tag was attached to the C-terminus of either the wild type (UNC93B-HA WT) or mutant (UNC93B-HA H412R) UNC93B protein was generated (FIG. 1a). Epitope-tagged wild type and mutant UNC93B were introduced into the macrophage cell line RAW 264.7 and conducted pulse-chase experiments. Wild type and mutant UNC93B-HA showed similar half-life (˜4 h), but exhibited distinct migration patterns in SDS-PAGE (FIG. 1b). Wild type UNC93B migrates as hetero-disperse material upon SDS-PAGE, whereas the UNC93B mutant form is dominated by a well-defined distinct polypeptide in addition to more diffuse material (FIG. 1b).

To rule out the possibility that the distinct polypeptide seen for mutant UNC93B represents a protein that associates with mutant UNC93B rather than the mutant protein itself, immunoprecipitation experiments were performed with the polyclonal antiserum directed against the C-terminal segment of UNC93B (anti-UNC-C), followed by re-immunoprecipitation experiments with an UNC93B antibody raised against the N-terminus (anti-UNC-N), anti-HA or anti-Flag antibodies. Epitope-tagged as well as endogenous UNC93B from RAW macrophages were recovered with the anti-UNC-C antiserum (FIG. 1c). Re-immunoprecipitation with anti-UNC-N antiserum after mild denaturation of the initial immunoprecipitation samples recovered endogenous as well as epitope-tagged UNC93B, whereas anti-HA and anti-Flag monoclonal antibodies recovered only epitope-tagged UNC93B proteins, as expected (FIG. 1c). The distinctly migrating polypeptide of mutant UNC93B was also recovered by re-immunoprecipitation (FIG. 1c), confirming that it derives from the UNC93B protein and not from a separate UNC93B-associated polypeptide.

To examine the maturation of endogenous wild type and mutant UNC93B proteins, pulse-chase analysis was performed of bone marrow-derived dendritic cells from wild type (C57BL/6) and UNC93B mutant (3d) mice. The 3d phenotype of the UNC93B mutant mice was confirmed through measurement of TNF production in BM-DCs after stimulation with TLR agonists. As reported, cells from 3d mice showed a complete lack of response to TLR7 and 9 agonists, whereas the response to the TLR4 agonist LPS was not affected (FIG. 7). The cells were first labeled with ³⁵S-methionine/cysteine for 30 min. After chase periods of 0, 1 and 4 hours, endogenous UNC93B was recovered by immunoprecipitation with the anti-UNC-C antiserum. The immunoprecipitates were then subjected to glycosidase digestion. In agreement with the proposed intracellular location of UNC93B as an ER-localized protein (Tabeta et al., 2006), it was found that both wild type and mutant UNC93B proteins recovered during the entire chase period retained full sensitivity to Endoglycosidase H (Endo H) (FIG. 1d). Immunofluorescence analysis of wild type and mutant UNC93B-HA with an anti-HA monoclonal antibody in paraformaldehyde-fixed RAW cells resulted in a reticular membrane staining pattern that closely overlaps with staining pattern for the ER membrane protein calnexin, confirming ER localization of both wild type and mutant UNC93B (data not shown).

Notably, the endogenous UNC93B mutant protein recovered from 3d mice showed the same discrete polypeptide in addition to the presence of diffuse material, whereas wild type UNC93B only showed the hetero-disperse band (FIG. 1d). As observed for epitope-tagged UNC93B, endogenous UNC93B is a stable protein and wild type and mutant UNC93B do not show any difference in stability. These results further show that tagging UNC93B at its C-terminus does not necessarily influence its maturation or stability.

From these experiments it appears that the phenotype observed in the UNC93B mutant mice does not result from the lack of UNC93B protein expression nor can it be attributed to a shortened half-life of the mutant UNC93B protein. Further, both wild type and mutant UNC93B retain full Endo H sensitivity, consistent with their localization to the ER.

### Example 2

### Maturation of MHC Products is Normal in BM-DCs from 3d Mice

The 3d mice not only show a defect in TLR signaling via TLRs 3, 7, and 9, but are also compromised in their ability to engage in cross-presentation via Class I MHC molecules and in Class II MHC-restricted antigen presentation (Tabeta et al., 2006). Even though surface levels of MHC products at steady state may not be affected by the 3d mutation, this leaves open the possibility of alterations in their trafficking. Pulse chase analysis was performed for Class I and Class II MHC products on BM-DCs obtained from wild type and 3d mice. Maturation of Class II MHC molecules was examined by assessing the levels of SDS-stable peptide-loaded αβ dimers, as well as the kinetics of SDS-stable dimer formation. The results from wild type and 3d mice were indistinguishable (FIG. 2). No difference in synthesis and maturation of Class I MHC molecules either was observed (data not shown). It appears that at least at this level of analysis, defects in MHC-restricted antigen presentation in 3d mice are unlikely to result from aberrant trafficking of MHC products.

### Example 3

### Immunoprecipitation of Wild Type UNC93B Recovers a Protein of ˜130 kDa that Retains Endo H Sensitivity

Since wild type and mutant UNC93B did not show any difference in localization, expression levels, maturation and stability, the effect of the point mutation may lie in the loss or gain of interaction partners of the UNC93B proteins. So far, no interacting proteins have been described for UNC93B.

To identify interacting proteins of UNC93B, pulse-chase experiments were conducted in RAW macrophages stably expressing epitope-tagged wild type or mutant UNC93B-HA (see FIG. 1a). Cells were lysed after 0, 90 or 180 min of chase in mild detergent (1% digitonin). Digestion with either Endo H or Peptide: N-glycosidase F (PNGase F) was used to monitor the glycosylation status of UNC93B-HA. As observed for endogenous UNC93B, both wild type and mutant UNC93B-HA retained Endo H and PNGase F sensitivity throughout the chase period (FIG. 3a, middle and right panel).

The presence of a polypeptide of lesser autoradiography intensity, and of a size (˜130 kDa) inconsistent with that predicted for UNC93B itself was noted. This polypeptide, too, retained full Endo H sensitivity. The presence of this additional polypeptide was observed only for wild type, but not for mutant UNC93B, even though the mutant protein was expressed at levels comparable to that of wild type UNC93B, as assessed by both immunoblotting (not shown) and immunoprecipitation on extracts of RAW macrophages stably expressing the relevant constructs (FIGS. 1b and 3a). The ability to recruit the ˜130 kDa polypeptide thus correlates with the functional properties of UNC93B. Communoprecipitation of a ˜130 kDa, Endo H-sensitive polypeptide with wild type but not with mutant UNC93B in A20 B cells was observed (FIG. 6d and data not shown). In A20 B cells, an additional polypeptide of ˜150 kDa was detected that had characteristics similar to that of the ˜130 kDa polypeptide, in that it was also Endo H-sensitive and was coimmunoprecipitated only with wild type UNC93B (FIG. 6d and data not shown).

### Example 4

### Large Scale Immunoprecipitation of UNC93B Identifies TLRs 3, 7, 9 and 13 as Interacting Proteins of Wild Type but not of Mutant UNC93B

To identify UNC93B associated polypeptides, large-scale cell cultures of RAW macrophages stably expressing either wild type or mutant UNC93B-HA were prepared, and a preparative immunoprecipitation of the tagged UNC93B proteins by retrieval via the HA epitope tag was conducted (FIG. 1a). Bound materials were released by digestion with TEV protease. After resolution of eluted polypeptides by SDS-PAGE and visualization by silver staining, polypeptides unique to wild type UNC93B bound material were excised, as well as polypeptides common to both wild type and mutant UNC93B samples, and determined their identity by mass spectrometry (FIG. 3b, Tables 2-4).

As expected, peptides for UNC93B were identified from both wild type and mutant UNC93B samples with good sequence coverage: 25.9% (10 peptides) and 11.7% (5 peptides), respectively (FIG. 3b, Table 2). Among the polypeptides coimmunoprecipitated only with wild type UNC93B, TLR3, TLR7, TLR9 and TLR13 were identified, with extensive sequence coverage over the entire length of the proteins (TLR3: 22.0%, 16 peptides; TLR7: 33.5%, 31 peptides; TLR9: 19.7%, 19 peptides; TLR13: 24.9%, 24 peptides) (FIG. 3b, Table 3). The same experimental approach was adapted to identify UNC93B interacting proteins in A20 B cells. Again, multiple peptides corresponding to TLR7 and TLR9 were identified from the sample for wild type UNC93B, but none from the sample for mutant UNC93B (data not shown). No peptides of TLRs 3, 7, 9 or 13 were recovered with mutant UNC93B, nor any peptide that corresponded to TLR4 or other TLRs with either wild type or mutant UNC93B in RAW macrophages or A20 B cells.

By coimmunoprecipitation and mass spectrometry, TLRs 3, 7, 9 and 13 have thus been identified as interacting partners for wild type UNC93B, and did not retrieve these TLRs with mutant UNC93B. This suggests that the H412R mutation within the transmembrane domain of UNC93B disrupts interaction between UNC93B and TLRs, and so abolishes TLR signaling.

TABLE 2
UNC93B WT

| protein | accession number | MW | peptide sequences | aa position |
|---|---|---|---|---|
| UNC93B | 17390915 | 66967,4 | HGVPDGPEAPLDELVGAYPNYNEEEEERR | 23-51 |
| | | | YGNMGLPDIDSK | 99-110 |
| | | | MLMGINVTPIAALLYTPVLIR | 111-131 |
| | | | YYEYSHYKEQDEQGPQQRPPR | 190-210 |
| | | | YYEYSHYK | 190-197 |
| | | | SQGILNGFNK | 264-273 |
| | | | PTEEIDLR | 313-320 |
| | | | SVGWGNIFQLPFK | 321-333 |
| | | | TGLSTLLGILYEDK | 451-464 |
| | | | TGLSTLLGILYEDKER | 451-466 |
| | | | LQQGLVPR | 518-525 |
| | | | KPCPYEQALGGDGPEEQ | 582-598 |

TABLE 3
UNC93B H412R

| protein | accession number | MW | peptide sequences | aa position |
|---|---|---|---|---|
| UNC93B | 17390915 | 66967,4 | YGNMGLPDIDSK | 99-110 |
| | | | YYEYSHYKEQDEQGPQQRPPR | 190-210 |
| | | | SVGWGNIFQLPFK | 321-333 |
| | | | TGLSTLLGILYEDK | 451-464 |
| | | | TGLSTLLGILYEDKER | 451-466 |
| | | | LQQGLVPR | 518-525 |

TABLE 4
UNC93B WT

| protein | accession | MW | peptide sequence | aa position |
|---|---|---|---|---|
| [symbol] | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| [symbol] | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| [symbol] | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| [symbol] | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |

[symbol]

indicates data missing or illegible when filed

### Example 5

### Wild Type UNC93B Interacts with TLR3 and 9, but not with TLR4

To confirm the interaction between wild type UNC93B and TLRs, and the failure of mutant UNC93B to associate with TLRs, myc-tagged TLR3, TLR4 and TLR9 fusion constructs were generated and co-expressed together with HA-tagged wild type and mutant UNC93B in HEK 293-T cells. Cells were lysed under mild conditions and subjected to immunoprecipitation with an anti-myc antibody. The presence of UNC93B in the myc immunoprecipitates was detected by immunoblot analysis with an anti-HA antibody (FIG. 4, upper panel). Wild type UNC93B interacts with TLR3 and TLR9 but did not interact with TLR4, confirming our observations made by the large scale coimmunoprecipitation and mass spectrometry (FIG. 3b). Mutant UNC93B failed to interact with TLRs 3, 4 and 9, mirroring our earlier results. Total lysates were analyzed for expression levels of wild type and mutant UNC93B by immunoblotting with an anti-HA antibody to confirm comparable expression levels (FIG. 4, lower panel).

### Example 6

### TLR3 and 9 Interact with Wild Type UNC93B Via their Transmembrane Regions

All TLRs consist of an extracellular domain with a series of leucine-rich repeats, a transmembrane domain and a cytosolic domain, which contains the conserved Toll-interleukin 1 receptor (TIR) domain. To address which region of the TLRs mediates binding to the UNC93B protein, myc-tagged versions of chimeric TLRs were generated (schematically depicted in FIG. 5a). Since TLR4 failed to bind to UNC93B, the transmembrane regions of TLR3 and TLR9 were exchanged with the transmembrane region of TLR4 (TLR3-4-3 and TLR9-4-9), and the transmembrane region of TLR4 was swapped for the transmembrane regions of TLR3 or TLR9 (TLR4-3-4 and TLR 4-9-4). These myc-tagged TLR chimeras and myc-tagged wild type TLRs 3, 4 and 9 were coexpressed with wild type UNC93B-HA. Cells were then metabolically labeled with ³⁵S-methionine/cysteine and lysed under mild conditions. From these lysates, immunoprecipitations were performed with an antibody to either the myc or the HA epitope. The immunoprecipitation with the anti-myc antibody shows the expression levels of the TLR wild type proteins (FIG. 5b) and chimeric proteins (FIG. 5c). By immunoprecipitating UNC93B via the HA-tag, TLRs 3 and 9, but not TLR4 were recovered (FIG. 5b), confirming our earlier observations (FIGS. 3b and 4). The chimeric TLRs containing the transmembrane domain of TLR4 (TLR3-4-3 and TLR9-4-9) failed to bind to UNC93B, whereas the TLR4 chimeras containing either the transmembrane region of TLR3 or of TLR9 (TLR4-3-4 or TLR4-9-4) were readily recovered with UNC93B (FIG. 5c). These results establish that TLR3 and TLR9 interact with the wild type UNC93B protein via their respective transmembrane regions.

### Example 7

### Endogenous Wild Type UNC93B and TLR7 Associate in BM-DCs

To extend our observations from transduced cells to primary cells, the interactions of UNC93B with TLRs in bone marrow-derived dendritic cells obtained from wild type (C57BL/6) and 3d mutant mice were analyzed. Immunoprecipitation was carried out with rabbit anti-UNC-C serum. Considerable quantities of endogenous UNC93B were recovered from both wild type and mutant dendritic cells (FIG. 6a), confirming the earlier observation that the 3d mutation does not compromise stability or steady state levels of the UNC93B protein. As seen for RAW cells transfected with epitope-tagged UNC93B, the presence of several coimmunoprecipitating polypeptides in the size range of TLRs in wild type, but not in mutant UNC93B immunoprecipitates were observed (FIG. 6a, left panel and FIG. 1d). The identity of one of the interacting proteins was revealed by denaturation of the primary anti-UNC-C immunoprecipitates, followed by a second round of immunoprecipitation (re-immunoprecipiation) with an anti-TLR7 antibody. In addition, dendritic cells obtained from TLR7 deficient mice were used as further evidence for the specificity of the anti-TLR7 antibody used. Wild type UNC93B immunoprecipitates contain TLR7, as seen from the re-immunoprecipitation experiment with the anti-TLR7 antibody (FIG. 6a, middle panel). As expected, no TLR7 is found in association with wild type UNC93B obtained from TLR7-deficient dendritic cells. In dendritic cells obtained from 3d mice no coimmunoprecipitation of TLR7 and UNC93B was observed. In addition, the additional polypeptides within the size range of TLRs that were coimmunoprecipitated with wild type UNC93B are absent from the 3d samples whereas they are still present in the sample from TLR7 deficient mice. Similar results were obtained when interactions between UNC93B and TLRs in splenocytes from wild type, 3d, TLR7^{-/-} and TLR9^{-/-} mice were analyzed. Again, TLR7 and TLR9 were co-immunoprecipitated only with wild type, but not with mutant UNC93B (FIG. 8).

The inability of the mutant UNC93B protein to interact with TLRs might be due to reduced expression of TLRs in cells from 3d mice. The inability of the 3d mice to signal via TLRs could therefore be the consequence of destabilization of TLRs, if UNC93B would serve a chaperone function. However, this is clearly not the case, since equivalent amounts of TLR7 were recovered by direct immunoprecipitation from both wild type and the 3d dendritic cell lysates using the TLR7 antibody (FIG. 6b).

In addition, the interaction between UNC93B and TLR7 was confirmed by first immunoprecipitating TLR7 and subsequently re-immunoprecipitating UNC93B from the denatured TLR7 immunoprecipitates. Wild type but not mutant UNC93B was recovered by immunoprecipitation of TLR7 (FIG. 6c).

In summary, the physical interaction of UNC93B with TLRs in primary cells was confirmed and it was shown that the mutant UNC93B protein no longer engages in such complex formation. Our earlier results described above obtained with the use of epitope-tagged versions of UNC93B are thus valid for endogenous proteins in primary dendritic cells and splenocytes of the appropriate genetic backgrounds.

### Example 8

### Activation of TLRs does not Affect the Interaction Between UNC93B and TLRs

To determine whether activation of TLRs with their respective agonists regulates the interaction with UNC93B, A20 B cells that stably express wild type or mutant UNC93B-HA were stimulated with TLR7 and 9 agonists: imiquimod or gardiquimod for TLR7 and CpG DNA for TLR9. After metabolically labeling cells with ³⁵S-methionine/cysteine, cell lysates were subjected to immunoprecipiation with an anti-HA antibody to retrieve UNC93B. No significant changes in the levels of coimmunoprecipitated TLR polypeptides were observed after TLR activation when compared to unstimulated cells, nor was additional interacting proteins for agonist-exposed cells observed (FIG. 6d). Similarly, stimulation with the TLR agonists did not alter the interaction between endogenous UNC93B and TLRs in splenocytes from wild type mice (data not shown).

### Materials and Methods for Examples 9-14

Reagents. The affinity-purified antibody against the C-terminal region of murine UNC93B has been described⁶. Imiquimod (R837) and poly (I:C) were purchased from Invivogen, CpG DNA (1826-CpG) and carboxytetramethylrhodamine (TAMRA)-conjugated CpG from TIB Molbiol, and LPS (E. coli 026:B6) and brefeldin A from Sigma. Endoglycosidase H (Endo H) was purchased from New England Biolabs and 3 μm polystyrene beads were from Bangs Laboratories. Anti-Lamp1 (clone 1D4B), anti-CD16 (clone 2.4G2), PE-conjugated anti-CD11c (clone HL3), PE-conjugated I-A^{b} (clone AF6-120.1) and Alexa Fluor 647-conjugated anti-TNF (clone MP6-XT22) antibodies were obtained from BD Biosciences.

Mice and Cell lines. Wild type C57BL/6 mice were purchased from Charles River Laboratories and 3d mice were obtained from Bruce Beutler (The Scripps Research Institute)⁴. Human embryonic kidney (HEK) cells 293T (ATCC CRL-11268) and RAW264.7 (ATCC TIB-71) were maintained in DMEM containing 10% heat-inactivated fetal calf serum (IFS) and penicillin/streptomycin (P/S).

Preparation of bone marrow-derived dendritic cells. BM-DCs were prepared from C57BL/6 and UNC93B mutant (3d) mice as described⁶.

DNA cloning. Murine UNC93B (BC018388) was C-terminally fused with the GFP or cherry tag¹⁷. The point mutant UNC93B (H412R) was generated by sequential PCR with primers carrying the point mutation CAC (His) to CGC (Arg) and C-terminally fused with the GFP or cherry tag. GFP or cherry tagged UNC93B WT and H412R were cloned into the retroviral pMSCVneo vector (Clontech). C-terminally GFP or cherry-tagged murine TLR7 and TLR9 were generated by PCR and cloned into the retroviral pMSCVpuro vector (Clontech). The chimeric TLR9/4 was constructed by PCR “sewing” with cDNA corresponding to the following amino acids of the TLRs: 1-810 of TLR9 and 626-835 of TLR4. TLR9/4 was C-terminally fused with GFP and cloned into the pMSCVpuro vector. UNC93B-GFP-Ist2p was generated by fusing the last 69 amino acid sequence of yeast Ist2p²⁴ to the C-terminus of UNC93B-GFP by PCR and cloned into pcDNA3.1 (Clontech). The Ist2p sequence was derived from a CD4-GFP-Ist2p construct (a gift from Matthias Seedorf, University of Heidelberg). Expression constructs TLR4-YFP and TLR7-YFP (both in the retroviral vector pMXrmv5) were kindly provided by Tadashi Nishiya (Hokkaido University, Japan)¹¹. The cherry-KDEL construct was generated by substituting the GFP sequence in pCMV/myc/ER/GFP (Clontech) with the cherry sequence by PCR. The cherry-KDEL construct was further subcloned into pMSCVneo by digestion with EcoR I and Sma I. All the constructs were verified by sequencing.

Retroviral transduction. HEK 293T cells were transfected with plasmids encoding VSV-G, Gag-Pol and pMSCV-UNC93B-GFP/cherry (WT), pMSCV-UNC93B-GFP/cherry (H412R), TLR9-GFP/cherry, TLR9/4-GFP, TLR4-YFP, or TLR7-GFP/YFP. 24 h and 48 h post transfection, medium containing viral particles was collected, filtered through a 0.45 μm membrane and added to RAW macrophages or BM-DCs at day 1 of BM-DC culture. The following day, cells were fed with fresh media.

Pulse-chase, immunoprecipitation and Endo H assay. In brief, five days old BM-DCs were starved for methionine and cysteine in Met/Cys-free DMEM (starvation medium) for 30 min, pulsed for 2 h with ³⁵S-labeled methionine/cysteine (Perkin Elmer) in starvation medium supplemented with dialyzed IFS, chased with an excess of non-radioactive amino acids in regular DMEM for 1 h and either non-stimulated or stimulated with TLR ligands for another hour in chase media (LPS: 1 μg/ml, poly (I:C): 50 μg/ml, Imiquimod: 2.5 μM, CpG DNA: 2.5 μM). Cells were lysed in lysis buffer (50 mM Tris-HCl, pH 7.4, 150 mM NaCl, 5 mM EDTA, 1% digitonin) supplemented with protease inhibitors (Roche). Lysates were equalized for incorporation of radioactive material with ³⁵S counts in the trichloroacetic acid precipitate and immunoprecipitated with the anti-UNC93B antibody or, as negative control, with normal rabbit serum (NRS). Washes were performed with the same buffer used for lysis but containing 0.1% digitonin. Immunoprecipitates were subjected to 10% SDS-PAGE without heating the samples and polypeptides were visualized by fluorography. Digestion with Endoglycosidase H was performed, where indicated, in accordance with the manufacturer's instructions.

TNF assay. Five days old BM-DCs expressing either wild type or mutant UNC93B-GFP were stimulated with 1 μg/ml LPS, 100 nM imiquimod or 1 μM CpG for 4 hours in the presence of 10 μg/ml brefeldin A. The cells were fixed with 4% formaldehyde for 10 min at RT and permeabilized with 0.5% saponin in FACS buffer (PBS with 2% BSA and 0.05% sodium azide) for 10 min. After blocking with anti-CD16 antibody (clone 2.4G2) for 30 min, cells were stained with PE-conjugated anti-CD11c (clone HL3) and Alexa Fluor 647-conjugated anti-TNF (clone MP6-XT22) antibodies for 30 min. Fluorescence intensity was measured with LSR I flow cytometer (BD biosciences). Data were collected with CellQuest (BD biosciences) and analyzed with FlowJo (TriStar).

Immunofluorescence staining. BM-DCs grown on coverglasses were fixed with 2% paraformaldehyde for 10 min at RT. Cells were permeabilized with 0.05% saponin in PBS/5% BSA and blocked for 30 min at RT. Cells were subsequently stained with a rabbit polyclonal anti-MHC class I (p8), a hamster monoclonal anti-MHC class II (clone N22) or a rat monoclonal anti-Lamp1 (clone 1D4B) antibody followed by Alexa Fluor-conjugated secondary antibodies (Invitrogen). After washes, coverglasses were mounted on a slideglass using Fluoromount G (Southern Biotech).

Image acquisition and data analysis. For live cell imaging, cells were grown in 8 well-chambered coverglasses (LAB-TEK). During imaging, cells were maintained in phenol red-free DMEM supplemented with 25 mM Hepes (pH 7.4) at 37° C. with 5% supplemental CO₂ in room air using a Solent scientific chamber. Images were acquired using a spinning disk confocal microscope equipped with a 3 watt water-cooled 5 line laser (Prairie Technologies), a Nikon TE2000-U inverted microscope, acousto-optic tunable filters (AOTF), a Yokagawa QLC-100 spinning disk confocal head, a Prior x,y stage controller, a Piezo z stage and Hamamatsu Orca ER and EM CCD cameras. All images were taken with a Nikon 100X 1.4NA DIC lens. Metamorph (Molecular Devices) was used for image acquisition and figures were constructed using Adobe Photoshop and Illustrator. Quantitative colocalization analyses were performed using applications ‘Measure Colocalization’ and ‘Correlation Plot’ in the MetaMorph software (Molecular Devices).

### Example 9

### UNC93B Specifically Interacts with Intracellular Nucleotide-Sensing TLRs and Regulates their Trafficking to Endolysosomes

UNC93B plays an essential role in signaling by the nucleotide-sensing TLRs^{4, 5, 15}, and a missense mutation in the Unc93b gene (H412R) abrogates signaling via TLRs 3, 7 and 9 without compromising other TLRs⁴. Functional null mutations of Unc93b are responsible for herpes simplex encephalitis in children⁵. As in 3d mice, cells from UNC93B-deficient patients are defective in signaling via the nucleotide-sensing TLRs 3, 7, 8 and 9⁵. As demonstrated above, UNC93B specifically binds to TLRs 3, 7, 9 and 13 via the transmembrane domains of the TLRs. The point mutation H412R of UNC93B in 3d mice abolishes these interactions⁶. The physical interactions between UNC93B and its client TLRs thus somehow control proper TLR signaling⁶.

The interactions between UNC93B and the nucleotide-sensing TLRs are stably maintained even after stimulation of various TLRs⁶ (FIG. 14a). Neither UNC93B nor its client TLRs ever acquire endo H resistance^{6, 12, 14}, even though they do so when cells are treated with brefeldin A (FIG. 14b). Therefore, applicants postulated that i) the UNC93B/TLR complex may reach lysosomes without passing through the Golgi and ii) the interaction between UNC93B and TLR9 controls TLR9 trafficking. To test this hypothesis, TLR9-GFP fusion protein was expressed in bone marrow-derived dendritic cells (BM-DCs) from wild type (B6) and UNC93B mutant (3d) mice and compared localization of TLR9 before and after stimulation with CpG DNA (ODN 1826). The majority of TLR9 is expressed in the ER of wild type cells^{12, 13} (FIG. 10 and FIG. 15b). In a small fraction of unstimulated wild type cells, TLR9 was also found in endosomes as well as in the ER (data not shown). Upon activation of wild type cells with CpG, TLR9 changed its localization from mostly ER to endosomes and tubular lysosomes, as inferred from colocalization with the late endosome/lysosomal marker proteins CD63 and Lamp1 (FIG. 10a, FIGS. 15c and 16a). In contrast, in 3d cells TLR9 remained in the ER and failed to translocate to endolysosomes, even after stimulation with CpG.

Like TLR9, TLR7 resides intracellularly^{9, 11}. To analyze subcellular localization of TLR7, TLR7-YFP fusion protein was expressed in BM-DCs. TLR7, like TLR9, was found to be localized in the ER of unstimulated DCs (FIG. 10b and FIG. 15a). Moreover, trafficking of TLR7 from the ER to endolysosomes was also observed when wild type cells were stimulated with the synthetic TLR7 agonist, imiquimod (FIG. 10b and FIG. 17). TLR7 is transported to endosomes also in response to stimulation with agonists that do not target TLR7 directly (FIG. 10c). However, in 3d cells TLR7 failed to exit the ER, similar to what was observed for TLR9. In contrast to TLR7 and TLR9, the localization of TLR4 at the cell surface is not affected by the 3d mutation, consistent with the ability of 3d cells to respond to LPS⁴ (FIG. 18). Therefore, UNC93B specifically interacts with intracellular nucleotide-sensing TLRs and regulates their trafficking to endolysosomes, from where the receptors transmit signals via MyD88/TRIF-dependent pathways^{8, 12}.

Although cells from 3d mice also have defects in cross-presentation and MHC class II-mediated antigen presentation^{4, 16}, no significant differences in localization of class I and class II MHC molecules in 3d cells was observed when compared to wild type cells (FIG. 19).

### Example 10

### Failure of Mutant UNC93B to Interact with TLRs Causes the TLR Trafficking Defects in 3d Cells

To confirm that the failure of mutant UNC93B to interact with TLRs causes the TLR trafficking defects in 3d cells, TLR9-GFP was coexpressed with wild type UNC93B fused to a red fluorescent cherry protein (UNC93B-cherry) in 3d cells¹⁷. Indeed, in 3d cells that express wild type UNC93B-cherry, TLR9 trafficking was normal: upon CpG stimulation, TLR9-GFP translocated properly into endolysosomes (FIG. 11a). Expression of mutant UNC93B-cherry did not rescue the TLR9 trafficking defect in 3d cells. Wild type UNC93B-cherry colocalized with TLR9 in endolysosomes (FIG. 11a). UNC93B thus travels to endolysosomes along with TLR9 when cells are stimulated.

TLR9 was also recruited to phagosomal membranes when BM-DCs from wild type mice were fed polystyrene beads (FIG. 11b). However, in 3d cells, TLR9 failed to translocate to phagosomes and remains in the ER, although 3d DCs efficiently phagocytosed the beads⁴. Expression of wild type but not mutant UNC93B in 3d cells restored trafficking of TLR9 to phagosomes (FIG. 11c). Wild type UNC93B-cherry colocalized with TLR9 in phagosomes. Therefore, UNC93B is required for proper trafficking of TLR9 to phagosomes as well as to endolysosomes. This trait may provide a link between the 3d mutation and the reported defect in cross-presentation⁴, a process favored by phagocytosis of antigen-positive materials¹⁸.

### Example 11

### Expression of Wild Type UNC93B in 3d BM-DCs Restores TLR Signaling Defects

BM-DCs from 3d cells failed to produce TNF when stimulated with imiquimod or CpG^{4, 6} (FIG. 11d). However, upon stimulation with TLR agonists, 3d cells that express wild type UNC93B produced as much TNF as did wild type cells⁴ (FIG. 11d). Expression of additional mutant UNC93B (H412R) in 3d cells did not restore the cytokine production in response to TLR7 or TLR9 activation. Expression of wild type or mutant UNC93B did not affect LPS-induced TNF production (FIG. 11d).

### Example 12

### Mutant UNC93B is Defective in its Own Trafficking

Unlike wild type UNC93B-cherry, mutant UNC93B-cherry showed a weak and diffuse labeling pattern in stimulated cells (FIG. 11a). To determine more accurately the localization of mutant UNC93B, wild type and mutant UNC93B-GFP fusion proteins were expressed in BM-DCs. Prior to stimulation, both wild type and mutant UNC93B were found in the ER⁴ (FIG. 12a and FIG. 20). When cells were stimulated with a dye-labeled CpG (TAMRA-CpG), wild type UNC93B-GFP colocalized with TAMRA-CpG in endolysosomes. Wild type UNC93B also colocalizes with Class II MHC and CD63 in CpG-stimulated cells (FIGS. 16b and 20). Moreover, endogenous wild type UNC93B colocalizes with Lamp1 when BM-DCs were stimulated with CpG (FIG. 20c). In contrast, mutant UNC93B-GFP did not translocate to endolysosomes upon stimulation with CpG (FIG. 12; FIGS. 16 and 20). Therefore, mutant UNC93B not only fails to interact with TLRs to deliver the receptors to endolysosomes, but is also defective in its own trafficking. Without wishing to be bound by any theory, it is likely that an as yet unidentified ER protein interacts with wild type UNC93B and facilitates trafficking of UNC93B/TLR complexes to endolysosomes. The H412R mutation, localized within a transmembrane segment of UNC93B, may cause disruption of such interaction(s) and result in mislocalization of both UNC93B and its client TLRs.

### Example 13

### UNC93B is Necessary and Sufficient to Determine Intracellular Localization of TLR9 Through Physical Interaction

The 3d mutation in UNC93B is a loss of function mutation. To provide positive evidence for the role of UNC93B in trafficking of TLRs, an aberrant localization of UNC93B was enforced and determined where TLRs would now traffic. Wild type UNC93B was delivered to the cell surface by engraftment of a dominant plasma membrane-sorting sequence—derived from the yeast membrane protein Ist2p—onto the cytosolic tail of UNC93B-GFP^{19,20}. When coexpressed with wild type UNC93B-GFP-Ist2p, both TLR9-cherry and UNC93B-GFP-Ist2p are targeted to the plasma membrane (FIG. 12). In contrast, TLR9-cherry remained in the ER when coexpressed with the H412R mutant UNC93B-GFP-Ist2p, even though the mutant UNC93B-GFP-Ist2p was detectable at the cell surface. These data establish that UNC93B is necessary and sufficient to determine intracellular localization of TLR9 through physical interaction.

### Example 14

### UNC93B is Dispensible for CpG Recognition or for Initiation of TLR Signaling Via Adaptor Recruitment

In order to determine whether UNC93B plays a role in ligand recognition and recruitment of signaling adaptors to TLRs, in addition to its role of determining intracellular localization of the nucleotide-sensing TLRs, a chimeric TLR was expressed with the TLR9 ectodomain fused to the transmembrane and cytosolic domain of TLR4 (TLR9/4) in BM-DCs. TLR9/4 no longer interacts with UNC93B because it lacks the TLR9 transmembrane domain (ref. 6 and data not shown). As reported, TLR9/4 resides at the cell surface¹⁴ (FIG. 13). When TLR9/4 was expressed in 3d BM-DCs, the mutant cells regained their responsiveness to CpG stimulation and produced TNF (FIG. 13). UNC93B is therefore dispensible for CpG recognition or for initiation of TLR signaling via adaptor recruitment. It appears that the signaling defects seen in 3d cells are solely due to the failure of the nucleotide-sensing TLRs to reach the endolysosomes, where they initiate MyD88/TRIF-dependent signaling pathways²¹⁻²³.

### SUMMARY

In summary, the findings described above demonstrate that UNC93B specifically controls trafficking of nucleotide-sensing TLRs. In 3d cells that express mutant UNC93B, unable to interact with its client TLRs, the nucleotide-sensing TLRs cannot leave the ER and fail to signal (FIG. 21). In contrast, trafficking and signaling of plasma membrane localized TLRs are not affected by the UNC93B mutation. As described herein, manipulation of the UNC93B/TLR interaction should thus allow specific regulation of signaling via nucleotide-sensing TLRs without compromising function of the other TLRs. New challenges lie in further defining the molecular nature of signals that promote intracellular TLR trafficking (FIG. 21). In addition, the unconventional means of delivery of TLRs 7 and 9 from the ER to endolysosomes by UNC93B constitute new pathways whose further probing will lead to a fuller understanding of the mechanisms of TLR trafficking and signal transduction.

### REFERENCES

### Examples 9-14

1. Janeway, C. A., Jr. & Medzhitov, R. Innate immune recognition. Annu Rev Immunol 20, 197-216 (2002).
2. Takeda, K., Kaisho, T. & Akira, S. Toll-like receptors. Annu Rev Immunol 21, 335-76 (2003).
3. Kawai, T. & Akira, S. TLR signaling. Cell Death Differ 13, 816-25 (2006).
4. Tabeta, K. et al. The Unc93b1 mutation 3d disrupts exogenous antigen presentation and signaling via Toll-like receptors 3, 7 and 9. Nat Immunol 7, 156-64 (2006).
5. Casrouge, A. et al. Herpes simplex virus encephalitis in human UNC-93B deficiency. Science 314, 308-12 (2006).
6. Brinkmann, M. M. et al. The interaction between the ER membrane protein UNC93B and TLR3, 7, and 9 is crucial for TLR signaling. J Cell Biol 177, 265-75 (2007).
7. Latz, E. et al. Lipopolysaccharide rapidly traffics to and from the Golgi apparatus with the toll-like receptor 4-MD-2-CD14 complex in a process that is distinct from the initiation of signal transduction. J Biol Chem 277, 47834-43 (2002).
8. Ahmad-Nejad, P. et al. Bacterial CpG-DNA and lipopolysaccharides activate Toll-like receptors at distinct cellular compartments. Eur J Immunol 32, 1958-68 (2002).
9. Nishiya, T. & DeFranco, A. L. Ligand-regulated chimeric receptor approach reveals distinctive subcellular localization and signaling properties of the Toll-like receptors. J Biol Chem 279, 19008-17 (2004).
10. Matsumoto, M. et al. Subcellular localization of Toll-like receptor 3 in human dendritic cells. J Immunol 171, 3154-62 (2003).
11. Nishiya, T., Kajita, E., Miwa, S. & Defranco, A. L. TLR3 and TLR7 are targeted to the same intracellular compartments by distinct regulatory elements. J Biol Chem 280, 37107-17 (2005).
12. Latz, E. et al. TLR9 signals after translocating from the ER to CpG DNA in the lysosome. Nat Immunol 5, 190-8 (2004).
13. Leifer, C. A. et al. TLR9 is localized in the endoplasmic reticulum prior to stimulation. J Immunol 173, 1179-83 (2004).
14. Barton, G. M., Kagan, J. C. & Medzhitov, R. Intracellular localization of Toll-like receptor 9 prevents recognition of self DNA but facilitates access to viral DNA. Nat Immunol 7, 49-56 (2006).
15. Conley, M. E. Immunodeficiency: UNC-93B gets a toll call. Trends Immunol 28, 99-101 (2007).
16. Janssen, E. et al. Efficient T cell activation via a Toll-Interleukin 1 Receptor-independent pathway. Immunity 24, 787-99 (2006).
17. Shaner, N. C. et al. Improved monomeric red, orange and yellow fluorescent proteins derived from Discosoma sp. red fluorescent protein. Nat Biotechnol 22, 1567-72 (2004).
18. Kovacsovics-Bankowski, M., Clark, K., Benacerraf, B. & Rock, K. L. Efficient major histocompatibility complex class I presentation of exogenous antigen upon phagocytosis by macrophages. Proc Natl Acad Sci USA 90, 4942-6 (1993).
19. Juschke, C., Ferring, D., Jansen, R. P. & Seedorf, M. A novel transport pathway for a yeast plasma membrane protein encoded by a localized mRNA. Curr Biol 14, 406-11 (2004).
20. Franz, A., Maass, K. & Seedorf, M. A complex peptide-sorting signal, but no mRNA signal, is required for the Sec-independent transport of Ist2 from the yeast ER to the plasma membrane. FEBS Lett 581, 401-5 (2007).
21. Schnare, M., Holt, A. C., Takeda, K., Akira, S. & Medzhitov, R. Recognition of CpG DNA is mediated by signaling pathways dependent on the adaptor protein MyD88. Curr Biol 10, 1139-42 (2000).
22. Hoebe, K. et al. Identification of Lps2 as a key transducer of MyD88-independent TIR signalling. Nature 424, 743-8 (2003).
23. Yamamoto, M. et al. Role of adaptor TRIF in the MyD88-independent toll-like receptor signaling pathway. Science 301, 640-3 (2003).
24. Juschke, C., Wachter, A., Schwappach, B. & Seedorf, M. SEC18/NSF-independent, protein-sorting pathway from the yeast cortical ER to the plasma membrane. J Cell Biol 169, 613-22 (2005).

### REFERENCES FOR EXAMPLES 1-8 AND DETAILED DESCRIPTION

Ackerman, A. L., Giodini, A., and Cresswell, P. (2006). A Role for the Endoplasmic Reticulum Protein Retrotranslocation Machinery during Crosspresentation by Dendritic Cells. Immunity.
Ackerman, A. L., Kyritsis, C., Tampe, R., and Cresswell, P. (2005). Access of soluble antigens to the endoplasmic reticulum can explain cross-presentation by dendritic cells. Nat Immunol 6, 107-113.
Ahmad-Nejad, P., Hacker, H., Rutz, M., Bauer, S., Vabulas, R. M., and Wagner, H. (2002). Bacterial CpG-DNA and lipopolysaccharides activate Toll-like receptors at distinct cellular compartments. Eur J Immunol 32, 1958-1968.
Akira, S., Takeda, K., and Kaisho, T. (2001). Toll-like receptors: critical proteins linking innate and acquired immunity. Nat Immunol 2, 675-680.
Alexopoulou, L., Holt, A. C., Medzhitov, R., and Flavell, R. A. (2001). Recognition of double-stranded RNA and activation of NF-kappaB by Toll-like receptor 3. Nature 413, 732-738.
Barton, G. M., Kagan, J. C., and Medzhitov, R. (2006). Intracellular localization of Toll-like receptor 9 prevents recognition of self DNA but facilitates access to viral DNA. Nat Immunol 7, 49-56.
Bauer, S., Kirschning, C. J., Hacker, H., Redecke, V., Hausmann, S., Akira, S., Wagner, H., and Lipford, G. B. (2001). Human TLR9 confers responsiveness to bacterial DNA via species-specific CpG motif recognition. Proc Natl Acad Sci USA 98, 9237-9242.
Beutler, B., Jiang, Z., Georgel, P., Crozat, K., Croker, B., Rutschmann, S., Du, X., and Hoebe, K. (2006). Genetic analysis of host resistance: Toll-like receptor signaling and immunity at large. Annu Rev Immunol 24, 353-389.
Casrouge, A., Zhang, S. Y., Eidenschenk, C., Jouanguy, E., Puel, A., Yang, K., Alcais, A., Picard, C., Mahfoufi, N., Nicolas, N., et al. (2006). Herpes simplex virus encephalitis in human UNC-93B deficiency. Science 314, 308-312.
Curran, A. R., and Engelman, D. M. (2003). Sequence motifs, polar interactions and conformational changes in helical membrane proteins. Curr Opin Struct Biol 13, 412-417.
de la Cruz, I. P., Levin, J. Z., Cummins, C., Anderson, P., and Horvitz, H. R. (2003). sup-9, sup-10, and unc-93 may encode components of a two-pore K+ channel that coordinates muscle contraction in Caenorhabditis elegans. J Neurosci 23, 9133-9145.
Diebold, S. S., Kaisho, T., Hemmi, H., Akira, S., and Reise Sousa, C. (2004). Innate antiviral responses by means of TLR7-mediated recognition of single-stranded RNA. Science 303, 1529-1531.
Funami, K., Matsumoto, M., Oshiumi, H., Akazawa, T., Yamamoto, A., and Seya, T. (2004). The cytoplasmic ‘linker region’ in Toll-like receptor 3 controls receptor localization and signaling. Int Immunol 16, 1143-1154.
Greenwald, I. S., and Horvitz, H. R. (1980). unc-93 (e1500): A behavioral mutant of Caenorhabditis elegans that defines a gene with a wild-type null phenotype. Genetics 96, 147-164.
Heil, F., Hemmi, H., Hochrein, H., Ampenberger, F., Kirschning, C., Akira, S., Lipford, G., Wagner, H., and Bauer, S. (2004). Species-specific recognition of single-stranded RNA via toll-like receptor 7 and 8. Science 303, 1526-1529.
Hemmi, H., Kaisho, T., Takeuchi, O., Sato, S., Sanjo, H., Hoshino, K., Horiuchi, T., Tomizawa, H., Takeda, K., and Akira, S. (2002). Small anti-viral compounds activate immune cells via the TLR7 MyD88-dependent signaling pathway. Nat Immunol 3, 196-200.
Hemmi, H., Takeuchi, O., Kawai, T., Kaisho, T., Sato, S., Sanjo, H., Matsumoto, M., Hoshino, K., Wagner, H., Takeda, K., and Akira, S. (2000). A Toll-like receptor recognizes bacterial DNA. Nature 408, 740-745.
Hoebe, K., Du, X., Georgel, P., Janssen, E., Tabeta, K., Kim, S. O., Goode, J., Lin, P., Mann, N., Mudd, S., et al. (2003). Identification of Lps2 as a key transducer of MyD88-independent TIR signalling. Nature 424, 743-748.
Hoshino, K., Sugiyama, T., Matsumoto, M., Tanaka, T., Saito, M., Hemmi, H., Ohara, O., Akira, S., and Kaisho, T. (2006). IkappaB kinase-alpha is critical for interferon-alpha production induced by Toll-like receptors 7 and 9. Nature 440, 949-953.
Imai, J., Hasegawa, H., Maruya, M., Koyasu, S., and Yahara, I. (2005). Exogenous antigens are processed through the endoplasmic reticulum-associated degradation (ERAD) in cross-presentation by dendritic cells. Int Immunol 17, 45-53.
Iwasaki, A., and Medzhitov, R. (2004). Toll-like receptor control of the adaptive immune responses. Nat Immunol 5, 987-995.
Janeway, C. A., Jr., and Medzhitov, R. (2002). Innate immune recognition. Annu Rev Immunol 20, 197-216.
Kajita, E., Nishiya, T., and Miwa, S. (2006). The transmembrane domain directs TLR9 to intracellular compartments that contain TLR3. Biochem Biophys Res Commun 343, 578-584.
Kashuba, V. I., Protopopov, A. I., Kvasha, S. M., Gizatullin, R. Z., Wahlestedt, C., Kisselev, L. L., Klein, G., and Zabarovsky, E. R. (2002). hUNC93B1: a novel human gene representing a new gene family and encoding an unc-93-like protein. Gene 283, 209-217.
Kawai, T., Adachi, O., Ogawa, T., Takeda, K., and Akira, S. (1999). Unresponsiveness of MyD88-deficient mice to endotoxin. Immunity 11, 115-122.
Kawai, T., and Akira, S. (2006). TLR signaling. Cell Death Differ 13, 816-825.
Latz, E., Schoenemeyer, A., Visintin, A., Fitzgerald, K. A., Monks, B. G., Knetter, C. F., Lien, E., Nilsen, N. J., Espevik, T., and Golenbock, D. T. (2004). TLR9 signals after translocating from the ER to CpG DNA in the lysosome. Nat Immunol 5, 190-198.
Leifer, C. A., Kennedy, M. N., Mazzoni, A., Lee, C., Kruhlak, M. J., and Segal, D. M. (2004). TLR9 is localized in the endoplasmic reticulum prior to stimulation. J Immunol 173, 1179-1183.
Levin, J. Z., and Horvitz, H. R. (1992). The Caenorhabditis elegans unc-93 gene encodes a putative transmembrane protein that regulates muscle contraction. J Cell Biol 117, 143-155.
Lilley, B. N., and Ploegh, H. L. (2004). A membrane protein required for dislocation of misfolded proteins from the ER. Nature 429, 834-840.
Liu, Y., Dodds, P., Emilion, G., Mungall, A. J., Dunham, I., Beck, S., Wells, R. S., Charnock, F. M., and Ganesan, T. S. (2002). The human homologue of unc-93 maps to chromosome 6q27-characterisation and analysis in sporadic epithelial ovarian cancer. BMC Genet. 3, 20.
Maehr, R., Hang, H. C., Mintern, J. D., Kim, Y. M., Cuvillier, A., Nishimura, M., Yamada, K., Shirahama-Noda, K., Hara-Nishimura, I., and Ploegh, H. L. (2005). Asparagine endopeptidase is not essential for class II MHC antigen presentation but is required for processing of cathepsin L in mice. J Immunol 174, 7066-7074.
Matsumoto, M., Funami, K., Tanabe, M., Oshiumi, H., Shingai, M., Seto, Y., Yamamoto, A., and Seya, T. (2003). Subcellular localization of Toll-like receptor 3 in human dendritic cells. J Immunol 171, 3154-3162.
Nagai, Y., Akashi, S., Nagafuku, M., Ogata, M., Iwakura, Y., Akira, S., Kitamura, T., Kosugi, A., Kimoto, M., and Miyake, K. (2002). Essential role of MD-2 in LPS responsiveness and TLR4 distribution. Nat Immunol 3, 667-672.
Nishiya, T., and DeFranco, A. L. (2004). Ligand-regulated chimeric receptor approach reveals distinctive subcellular localization and signaling properties of the Toll-like receptors. J Biol Chem 279, 19008-19017.
Nishiya, T., Kajita, E., Miwa, S., and Defranco, A. L. (2005). TLR3 and TLR7 are targeted to the same intracellular compartments by distinct regulatory elements. J Biol Chem 280, 37107-37117.
Partridge, A. W., Therien, A. G., and Deber, C. M. (2004). Missense mutations in transmembrane domains of proteins: phenotypic propensity of polar residues for human disease. Proteins 54, 648-656.
Poltorak, A., He, X., Smimova, I., Liu, M. Y., Van Huffel, C., Du, X., Birdwell, D., Alejos, E., Silva, M., Galanos, C., et al. (1998). Defective LPS signaling in C3H/HeJ and C57BL/10ScCr mice: mutations in Tlr4 gene. Science 282, 2085-2088.
Randow, F., and Seed, B. (2001). Endoplasmic reticulum chaperone gp96 is required for innate immunity but not cell viability. Nat Cell Biol 3, 891-896.
Shim, J. H., Xiao, C., Paschal, A. E., Bailey, S. T., Rao, P., Hayden, M. S., Lee, K. Y., Bussey, C., Steckel, M., Tanaka, N., et al. (2005). TAK1, but not TAB1 or TAB2, plays an essential role in multiple signaling pathways in vivo. Genes Dev 19, 2668-2681.
Shimazu, R., Akashi, S., Ogata, H., Nagai, Y., Fukudome, K., Miyake, K., and Kimoto, M. (1999). MD-2, a molecule that confers lipopolysaccharide responsiveness on Toll-like receptor 4. J Exp Med 189, 1777-1782.
Spooner, R. A., Smith, D. C., Easton, A. J., Roberts, L. M., and Lord, J. M. (2006). Retrograde transport pathways utilised by viruses and protein toxins. Virol J 3, 26.
Suzuki, N., Suzuki, S., Duncan, G. S., Millar, D. G., Wada, T., Mirtsos, C., Takada, H., Wakeham, A., Itie, A., Li, S., et al. (2002). Severe impairment of interleukin-1 and Toll-like receptor signalling in mice lacking IRAK-4. Nature 416, 750-756.
Tabeta, K., Georgel, P., Janssen, E., Du, X., Hoebe, K., Crozat, K., Mudd, S., Shamel, L., Sovath, S., Goode, J., et al. (2004). Toll-like receptors 9 and 3 as essential components of innate immune defense against mouse cytomegalovirus infection. Proc Natl Acad Sci USA 101, 3516-3521.
Tabeta, K., Hoebe, K., Janssen, E. M., Du, X., Georgel, P., Crozat, K., Mudd, S., Mann, N., Sovath, S., Goode, J., et al. (2006). The Unc93b1 mutation 3d disrupts exogenous antigen presentation and signaling via Toll-like receptors 3, 7 and 9. Nat Immunol 7, 156-164.
Takeda, K., Kaisho, T., and Akira, S. (2003). Toll-like receptors. Annu Rev Immunol 21, 335-376.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

Certain claims are presented in dependent form for the sake of convenience, but Applicant reserves the right to rewrite any dependent claim in independent format to include the limitations of the independent claim and any other claim(s) on which such claim depends, and such rewritten claim is to be considered equivalent in all respects to the dependent claim in whatever form it is in (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited.

We claim:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A method of identifying an immunomodulator comprising the steps of:
(a) performing an interaction-specific assay that assesses the ability of a candidate agent to modulate interaction between an UNC93B polypeptide and an UNC93B-dependent TLR polypeptide;
(b) identifying the candidate agent as an immunomodulator if it modulates the interaction between the UNC93B polypeptide and the UNC93B-dependent TLR polypeptide.

26. (canceled)

27. (canceled)

28. (canceled)

29. (canceled)

30. The method of claim 24, which comprises steps of:
(a) providing a composition comprising an UNC93B polypeptide, an UNC93B-dependent TLR polypeptide, and a candidate agent;
(b) contacting the composition with a candidate agent under conditions suitable for a physical interaction to occur between the UNC93B polypeptide and the TLR polypeptide; and
(c) determining whether the agent alters the physical interaction that would, in the absence of the candidate agent, be expected to occur between the UNC93B polypeptide and the TLR polypeptide.

31. The method of claim 24, which comprises contacting cells that express the UNC93B polypeptide and the UNC93B-dependent TLR polypeptide with the candidate agent.

32. (canceled)

33. The method of claim 24, wherein the TLR is selected from the group consisting of: TLR3, TLR7, TLR8, and TLR9.

34. The method of claim 24, wherein the method comprises steps of:
(a) contacting a cell that expresses both an UNC93B polypeptide and an UNC93B-dependent TLR polypeptide with a candidate agent; and
(b) determining whether the candidate agent affects the localization of the UNC93B polypeptide or the TLR polypeptide, wherein an agent that affects the localization of the UNC93B polypeptide or the TLR polypeptide is identified as a potential immunomodulator that modulates signaling by an UNC93B-dependent TLR.

35. The method of claim 34 wherein an agent that reduces the amount of the UNC93B or TLR polypeptide in endosomes/lysosomes/phagosomes is identified as an agent that inhibits a physical interaction between UNC93B and the TLR.

36. The method of claim 34, wherein an agent that reduces the amount of the UNC93B or TLR polypeptide in endosomes/lysosomes/phagosomes is identified as an agent that inhibits a physical interaction between UNC93B and the TLR.

37. The method of claim 34, wherein an agent that increases the amount of the UNC93B or TLR polypeptide in endosomes/lysosomes/phagosomes is identified as an agent that enhances a physical interaction between UNC93B and the TLR.

38. The method of claim 34, wherein the UNC93B polypeptide or the TLR polypeptide is detectably labeled.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. A method of identifying an immunomodulator that modulates signaling by an UNC93B dependent TLR, comprising the steps of:
(a) contacting a cell that expresses both an UNC93B polypeptide and an UNC93B-dependent TLR polypeptide with a candidate agent; and
(b) determining whether the candidate agent affects the localization of the UNC93B polypeptide or the TLR polypeptide, wherein an agent that affects the localization of the UNC93B polypeptide or the TLR polypeptide is identified as a potential immunomodulator that modulates signaling by an UNC93B-dependent TLR.

55. The method of claim 54, wherein the UNC93B polypeptide or TLR polypeptide is detectably labeled.

56. The method of claim 54, wherein the cell is contacted with a stimulus for the TLR signaling pathway.

57. The method of claim 55, wherein the stimulus is a ligand for a nucleotide-sensing TLR.

58. The method of claim 54, wherein an agent that reduces the amount of the UNC93B or TLR polypeptide in endosomes/lysosomes/phagosomes is identified as an agent that inhibits a physical interaction between UNC93B and the TLR.

59. The method of claim 54, wherein an agent that increases the amount of the UNC93B or TLR polypeptide in endosomes/lysosomes/phagosomes is identified as an agent that enhances a physical interaction between UNC93B and the TLR.

60. The method of claim 54, wherein an agent that reduces the amount of the UNC93B or TLR polypeptide in endosomes/lysosomes/phagosomes is identified as an agent that inhibits signaling by the TLR.

61. The method of claim 54, wherein an agent that increases the amount of the UNC93B or TLR polypeptide in endosomes/lysosomes/phagosomes is identified as an agent that enhances signaling by the TLR.

62. The method of claim 54, wherein the method comprises assessing colocalization of the UNC93B polypeptide or the TLR polypeptide with an endosomal/lysosomal/phagosomal marker.

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. The method of claim 54, further comprising assessing the ability of the agent to alter cytokine release by the cell in response to a stimulus for the TLR signaling pathway.

75. (canceled)

76. (canceled)

77. (canceled)

78. (canceled)

79. A method of identifying an agent that modulates signaling by an UNC93B-dependent TLR, comprising steps of:
(a) contacting a cell that expresses an UNC93B polypeptide and an UNC93B-dependent TLR polypeptide with a candidate agent; and
(b) determining whether the candidate agent affects the localization of the UNC93B polypeptide or the TLR polypeptide, wherein an agent that affects the localization of the UNC93B polypeptide or the TLR polypeptide is identified as an agent that modulates signaling by the UNC93B-dependent TLR.

80. The method of claim 79, wherein the UNC93B polypeptide or TLR polypeptide is detectably labeled.

81. The method of claim 79, wherein the UNC93B polypeptide or TLR polypeptide comprises a fluorescent or luminescent domain.

82. (canceled)

83. (canceled)

84. (canceled)

85. (canceled)

86. The method of claim 54, wherein the UNC93B-dependent TLR is TLR9.

87. The method of claim 79, wherein the UNC93B-dependent TLR is TLR9.


SEQUENCE LISTING (136 sequences)

SEQ ID NO: 1 - protein, 597 aa, Homo sapiens
MEAEPPLYPMAGAAGPQGDEDLLGVPDGPEAPLDELVGAYPNYNEEEEERRYYRRKRLGVLKNVLAASAGGMLTYGVYLGLLQMQLILHYDETYREVKYGNMGLPDIDSKMLMGINVTPIAALLYTPVLIRFFGTKWMMFLAVGIYALFVSTNYWERYYTLVPSAVALGMAIVPLWASMGNYITRMAQKYHEYSHYKEQDGQGMKQRPPRGSHAPYLLVFQAIFYSFFHLSFACAQLPMIYFLNHYLYDLNHTLYNVQSCGTNSHGILSGFNKTVLRTLPRSGNLIVVESVLMAVAFLAMLLVLGLCGAAYRPTEEIDLRSVGWGNIFQLPFKHVRDYRLRHLVPFFIYSGFEVLFACTGIALGYGVCSVGLERLAYLLVAYSLGASAASLLGLLGLWLPRPVPLVAGAGVHLLLTFILFFWAPVPRVLQHSWILYVAAALWGVGSALNKTGLSTLLGILYEDKERQDFIFTIYHWWQAVAIFTVYLGSSLHMKAKLAVLLVTLVAAAVSYLRMEQKLRRGVAPRQPRIPRPQHKVRGYRYLEEDNSDESDAEGEHGDGAEEEAPPAGPRPGPEPAGLGRRPCPYEQAQGGDGPEEQ

SEQ ID NO: 2 - protein, 598 aa, Mus musculus
MEVEPPLYPVAGAAGPQGDEDRHGVPDGPEAPLDELVGAYPNYNEEEEERRYYRRKRLGVVKNVLAASTGVTLTYGVYLGLLQMQLILHYDETYREVKYGNMGLPDIDSKMLMGINVTPIAALLYTPVLIRFFGTKWMMFLAVGIYALFVSTNYWERYYTLVPSAVALGMAIVPLWASMGNYITRMSQKYYEYSHYKEQDEQGPQQRPPRGSHAPYLLVFQAIFYSFFHLSFACAQLPMIYFLNNYLYDLNHTLINVQSCGTKSQGILNGFNKTVLRTLPRSKNLIVVESVLMAVAFLAMLMVLGLCGAAYRPTEEIDLRSVGWGNIFQLPFKHVRDFRLRHLVPFFIYSGFEVLFACTGFALGYGVCSMGLERLAYLLIAYSLGASASSVLGLLGLWLPRSVPLVAGAGLHLLLTLSLFFWAPAPRVLQHSWIFYFVAALWGVGSALNKTGLSTLLGILYEDKERQDFIFTIYHWWQAVAIFVVYLGSSLPMKAKLAVLLVTLVAAAASYLWMEQKLQQGLVPRQPRIPKPQHKVRGYRYLEEDNSDESDMEGEQGQGDCAEDEAPQAGPLGTEPAGPCRKPCPYEQALGGDGPEEQ

SEQ ID NO: 3 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 4 - protein, 905 aa, Mus musculus
MKGCSSYLMYSFGGLLSLWILLVSSTNQCTVRYNVADCSHLKLTHIPDDLPSNITVLNLTHNQLRRLPPTNFTRYSQLAILDAGFNSISKLEPELCQILPLLKVLNLQHNELSQISDQTFVFCTNLTELDLMSNSIHKIKSNPFKNQKNLIKLDLSHNGLSSTKLGTGVQLENLQELLLAKNKILALRSEELEFLGNSSLRKLDLSSNPLKEFSPGCFQTIGKLFALLLNNAQLNPHLTEKLCWELSNTSIQNLSLANNQLLATSESTFSGLKWTNLTQLDLSYNNLHDVGNGSFSYLPSLRYLSLEYNNIQRLSPRSFYGLSNLRYLSLKRAFTKQSVSLASHPNIDDFSFQWLKYLEYLNMDDNNIPSTKSNTFTGLVSLKYLSLSKTFTSLQTLTNETFVSLAHSPLLTLNLTKNHISKIANGTFSWLGQLRILDLGLNEIEQKLSGQEWRGLRNIFEIYLSYNKYLQLSTSSFALVPSLQRLMLRRVALKNVDISPSPFRPLRNLTILDLSNNNIANINEDLLEGLENLEILDFQHNNLARLWKRANPGGPVNFLKGLSHLHILNLESNGLDEIPVGVFKNLFELKSINLGLNNLNKLEPFIFDDQTSLRSLNLQKNLITSVEKDVFGPPFQNLNSLDMRFNPFDCTCESISWFVNWINQTHTNISELSTHYLCNTPHHYYGFPLKLFDTSSCKDSAPFELLFIISTSMLLVFILVVLLIHIEGWRISFYWNVSVHRILGFKEIDTQAEQFEYTAYIIHAHKDRDWVWEHFSPMEEQDQSLKFCLEERDFEAGVLGLEAIVNSIKRSRKIIFVITHHLLKDPLCRRFKVHHAVQQAIEQNLDSIILIFLQNIPDYKLNHALCLRRGMFKSHCILNWPVQKERINAFHHKLQVALGSRNSAH

SEQ ID NO: 5 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 6 - protein, 1050 aa, Mus musculus
MVFSMWTRKRQILIFLNMLLVSRVFGFRWFPKTLPCEVKVNIPEAHVIVDCTDKHLTEIPEGIPTNTTNLTLTINHIPSISPDSFRRLNHLEEIDLRCNCVPVLLGSKANVCTKRLQIRPGSFSGLSDLKALYLDGNQLLEIPQDLPSSLHLLSLEANNIFSITKENLTELVNIETLYLGQNCYYRNPCNVSYSIEKDAFLVMRNLKVLSLKDNNVTAVPTTLPPNLLELYLYNNIIKKIQENDFNNLNELQVLDLSGNCPRCYNVPYPCTPCENNSPLQIHDNAFNSLTELKVLRLHSNSLQHVPPTWFKNMRNLQELDLSQNYLAREIEEAKFLHFLPNLVELDFSFNYELQVYHASITLPHSLSSLENLKILRVKGYVFKELKNSSLSVLHKLPRLEVLDLGTNFIKIADLNIFKHFENLKLIDLSVNKISPSEESREVGFCPNAQTSVDRHGPQVLEALHYFRYDEYARSCRFKNKEPPSFLPLNADCHIYGQTLDLSRNNIFFIKPSDFQHLSFLKCLNLSGNTIGQTLNGSELWPLRELRYLDFSNNRLDLLYSTAFEELQSLEVLDLSSNSHYFQAEGITHMLNFTKKLRLLDKLMMNDNDISTSASRTMESDSLRILEFRGNHLDVLWRAGDNRYLDFFKNLFNLEVLDISRNSLNSLPPEVFEGMPPNLKNLSLAKNGLKSFFWDRLQLLKHLEILDLSHNQLTKVPERLANCSKSLTTLILKHNQIRQLTKYFLEDALQLRYLDISSNKIQVIQKTSFPENVLNNLEMLVLHHNRFLCNCDAVWFVWWVNHTDVTIPYLATDVTCVGPGAHKGQSVISLDLYTCELDLTNLILFSVSISSVLFLMVVMTTSHLFFWDMWYIYYFWKAKIKGYQHLQSMESCYDAFIVYDTKNSAVTEWVLQELVAKLEDPREKHFNLCLEERDWLPGQPVLENLSQSIQLSKKTVFVMTQKYAKTESFKMAFYLSHQRLLDEKVDVIILIFLEKPLQKSKFLQLRKRLCRSSVLEWPANPQAHPYFWQCLKNALTTDNHVAYSQMFKETV

SEQ ID NO: 7 - protein, 1059 aa, Homo sapiens
MKESSLQNSSCSLGKETKKENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 8 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 9 - protein, 1032 aa, Mus musculus
MENMPPQSWILTCFCLLSSGTSAIFHKANYSRSYPCDEIRHNSLVIAECNHRQLHEVPQTIGKYVTNIDLSDNAITHITKESFQKLQNLTKIDLNHNAKQQHPNENKNGMNITEGALLSLRNLTVLLLEDNQLYTIPAGLPESLKELSLIQNNIFQVTKNNTFGLRNLERLYLGWNCYFKCNQTFKVEDGAFKNLIHLKVLSLSFNNLFYVPPKLPSSLRKLFLSNAKIMNITQEDFKGLENLTLLDLSGNCPRCYNAPFPCTPCKENSSIHIHPLAFQSLTQLLYLNLSSTSLRTIPSTWFENLSNLKELHLEFNYLVQEIASGAFLTKLPSLQILDLSFNFQYKEYLQFINISSNFSKLRSLKKLHLRGYVFRELKKKHFEHLQSLPNLATINLGINFIEKIDFKAFQNFSKLDVIYLSGNRIASVLDGTDYSSWRNRLRKPLSTDDDEFDPHVNFYHSTKPLIKPQCTAYGKALDLSLNNIFIIGKSQFEGFQDIACLNLSFNANTQVFNGTEFSSMPHIKYLDLTNNRLDFDDNNAFSDLHDLEVLDLSHNAHYFSIAGVTHRLGFIQNLINLRVLNLSHNGIYTLTEESELKSISLKELVFSGNRLDHLWNANDGKYWSIFKSLQNLIRLDLSYNNLQQIPNGAFLNLPQSLQELLISGNKLRFFNWTLLQYFPHLHLLDLSRNELYFLPNCLSKFAHSLETLLLSHNHFSHLPSGFLSEARNLVHLDLSFNTIKMINKSSLQTKMKTNLSILELHGNYFDCTCDISDFRSWLDENLNITIPKLVNVICSNPGDQKSKSIMSLDLTTCVSDTTAAVLFFLTFLTTSMVMLAALVHHLFYWDVWFIYHMCSAKLKGYRTSSTSQTFYDAYISYDTKDASVTDWVINELRYHLEESEDKSVLLCLEERDWDPGLPIIDNLMQSINQSKKTIFVLTKKYAKSWNFKTAFYLALQRLMDENMDVIIFILLEPVLQYSQYLRLRQRICKSSILQWPNNPKAENLFWQSLKNVVLTENDSRYDDLYIDSIRQY

SEQ ID NO: 10 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 11 - protein, 1032 aa, Mus musculus
MVLRRRTLHPLSLLVQAAVLAETLALGTLPAFLPCELKPHGLVDCNWLFLKSVPRFSAAASCSNITRLSLISNRIHHLHNSDFVHLSNLRQLNLKWNCPPTGLSPLHFSCHMTIEPRTFLAMRTLEELNLSYNGITTVPRLPSSLVNLSLSHTNILVLDANSLAGLYSLRVLFMDGNCYYKNPCTGAVKVTPGALLGLSNLTHLSLKYNNLTKVPRQLPPSLEYLLVSYNLIVKLGPEDLANLTSLRVLDVGGNCRRCDHAPNPCIECGQKSLHLHPETFHHLSHLEGLVLKDSSLHTLNSSWFQGLVNLSVLDLSENFLYESINHTNAFQNLTRLRKLNLSFNYRKKVSFARLHLASSFKNLVSLQELNMNGIFFRSLNKYTLRWLADLPKLHTLHLQMNFINQAQLSIFGTFRALRFVDLSDNRISGPSTLSEATPEEADDAEQEELLSADPHPAPLSTPASKNFMDRCKNFKFTMDLSRNNLVTIKPEMFVNLSRLQCLSLSHNSIAQAVNGSQFLPLTNLQVLDLSHNKLDLYHWKSFSELPQLQALDLSYNSQPFSMKGIGHNFSFVTHLSMLQSLSLAHNDIHTRVSSHLNSNSVRFLDFSGNGMGRMWDEGGLYLHFFQGLSGLLKLDLSQNNLHILRPQNLDNLPKSLKLLSLRDNYLSFFNWTSLSFLPNLEVLDLAGNQLKALTNGTLPNGTLLQKLDVSSNSIVSVVPAFFALAVELKEVNLSHNILKTVDRSWFGPIVMNLTVLDVRSNPLHCACGAAFVDLLLEVQTKVPGLANGVKCGSPGQLQGRSIFAQDLRLCLDEVLSWDCFGLSLLAVAVGMVVPILHHLCGWDVWYCFHLCLAWLPLLARSRRSAQTLPYDAFVVFDKAQSAVADWVYNELRVRLEERRGRRALRLCLEDRDWLPGQTLFENLWASIYGSRKTLFVLAHTDRVSGLLRTSFLLAQQRLLEDRKDVVVLVILRPDAHRSRYVRLRQRLCRQSVLFWPQQPNGQGGFWAQLSTALTRDNRHFYNQNFCRGPTAE

SEQ ID NO: 12 - protein, 991 aa, Mus musculus
MSGLYRILVQLEQSPYVKTVPLNMRRDFFFLVVTWMPKTVKMNGSSFVPSLQLLLMLVGFSLPPVAETYGFNKCTQYEFDIHHVLCIRKKITNLTEAISDIPRYTTHLNLTHNEIQVLPPWSFTNLSALVDLRLEWNSIWKIDEGAFRGLENLTLLNLVENKIQSVNNSFEGLSSLKTLLLSHNQITHIHKDAFTPLIKLKYLSLSRNNISDFSGILEAVQHLPCLERLDLTNNSIMYLDHSPRSLVSLTHLSFEGNKLRELNFSALSLPNLTNLSASRNGNKVIQNVYLKTLPQLKSLNLSGTVIKLENLSAKHLQNLRAMDLSNWELRHGHLDMKTVCHLLGNLPKLETLVFQKNVTNAEGIKQLAKCTRLLFLDLGQNSDLIYLNDSEFNALPSLQKLNLNKCQLSFINNRTWSSLQNLTSLDLSHNKFKSFPDFAFSPLKHLEFLSLSRNPITELNNLAFSGLFALKELNLAACWIVTIDRYSFTQFPNLEVLDLGDNNIRTLNHGTFRPLKKLQSLILSHNCLKILEPNSFSGLTNLRSLDLMYNSLSYFHEHLFSGLEKLLILKLGFNKITYETTRTLQYPPFIKLKSLKQLNLEGQRHGIQVVPSNFFQGLGSLQELLLGKNPSVFLDHHQFDPLINLTKLDISGTKDGDRSLYLNASLFQNLKRLKILRLENNNLESLVPDMFSSLQSLQVFSLRFNNLKVINQSHLKNLKSLMFFDVYGNKLQCTCDNLWFKNWSMNTEEVHIPFLRSYPCQQPGSQSLLIDFDDAMCNFDLGKVYFLCSFSMVLSTMVFSWFSTKMIASLWYGLYICRAWYLTKWHKTEKKFLYDAFVSFSATDEAWVYKELVPALEQGSQTTFKLCLHQRDFEPGIDIFENIQNAINTSRKTLCVVSNHYLHSEWCRLEVQLASMKMFYEHKDVIILIFLEEIPNYKLSSYHRLRKLINKQTFITWPDSVHQQPLFWARIRNALGKETVEKENTHLIVVE

SEQ ID NO: 13 - protein, 4 aa, Artificial Sequence
  Description of Artificial Sequence Synthetic peptide
KDEL

SEQ ID NO: 14 - protein, 30 aa, Artificial Sequence
  Description of Artificial Sequence Synthetic peptide
GSGSGSGSGSGSGSGSGSGSGSGSGSGSGS

SEQ ID NO: 15 - protein, 45 aa, Artificial Sequence
  Description of Artificial Sequence Synthetic peptide
TSPTSPTSPTSPTSPTSPTSPTSPTSPTSPTSPTSPTSPTSPTSP

SEQ ID NO: 16 - protein, 45 aa, Artificial Sequence
  Description of Artificial Sequence Synthetic peptide
GGGGGGGGGGGGGGGGGGGGGGGGGGGGGGGGGGGGGGGGGGGGG

SEQ ID NO: 17 - protein, 30 aa, Artificial Sequence
  Description of Artificial Sequence Synthetic peptide
GAGAGAGAGAGAGAGAGAGAGAGAGAGAGA

SEQ ID NO: 18 - protein, 30 aa, Artificial Sequence
  Description of Artificial Sequence Synthetic peptide
EKEKEKEKEKEKEKEKEKEKEKEKEKEKEK

SEQ ID NO: 19 - protein, 6 aa, Artificial Sequence
  Description of Artificial Sequence Synthetic 6xHis tag
HHHHHH

SEQ ID NO: 20 - protein, 16 aa, Artificial Sequence
  Description of Artificial Sequence Synthetic peptide
CDRHGVPDGPEAPLDE

SEQ ID NO: 21 - protein, 16 aa, Artificial Sequence
  Description of Artificial Sequence Synthetic peptide
CPDGPEAPLDELVGAY

SEQ ID NO: 22 - protein, 20 aa, Artificial Sequence
  Description of Artificial Sequence Synthetic peptide
CGAYPNYNEEEEERRYYRRK

SEQ ID NO: 23 - protein, 16 aa, Artificial Sequence
  Description of Artificial Sequence Synthetic peptide
CLQQGLVPRQPRIPKP

SEQ ID NO: 24 - protein, 16 aa, Artificial Sequence
  Description of Artificial Sequence Synthetic peptide
CRYLEEDNSDESDMEG

SEQ ID NO: 25 - protein, 15 aa, Artificial Sequence
  Description of Artificial Sequence Synthetic peptide
CPYEQALGGDGPEEQ

SEQ ID NO: 26 - protein, 7 aa, Tobacco Etch Virus
ENLYFQG

SEQ ID NO: 27 - protein, 29 aa, Mus musculus
HGVPDGPEAPLDELVGAYPNYNEEEEERR

SEQ ID NO: 28 - protein, 12 aa, Mus musculus
YGNMGLPDIDSK

SEQ ID NO: 29 - protein, 21 aa, Mus musculus
MLMGINVTPIAALLYTPVLIR

SEQ ID NO: 30 - protein, 21 aa, Mus musculus
YYEYSHYKEQDEQGPQQRPPR

SEQ ID NO: 31 - protein, 8 aa, Mus musculus
YYEYSHYK

SEQ ID NO: 32 - protein, 10 aa, Mus musculus
SQGILNGFNK

SEQ ID NO: 33 - protein, 8 aa, Mus musculus
PTEEIDLR

SEQ ID NO: 34 - protein, 13 aa, Mus musculus
SVGWGNIFQLPFK

SEQ ID NO: 35 - protein, 14 aa, Mus musculus
TGLSTLLGILYEDK

SEQ ID NO: 36 - protein, 16 aa, Mus musculus
TGLSTLLGILYEDKER

SEQ ID NO: 37 - protein, 8 aa, Mus musculus
LQQGLVPR

SEQ ID NO: 38 - protein, 17 aa, Mus musculus
KPCPYEQALGGDGPEEQ

SEQ ID NO: 39 - protein, 10 aa, Mus musculus
YNVADCSHLK

SEQ ID NO: 40 - protein, 13 aa, Mus musculus
LEPELCQILPLLK

SEQ ID NO: 41 - protein, 12 aa, Mus musculus
LDLSHNGLSSTK

SEQ ID NO: 42 - protein, 10 aa, Mus musculus
KLDLSSNPLK

SEQ ID NO: 43 - protein, 11 aa, Mus musculus
YLSLEYNNIQR

SEQ ID NO: 44 - protein, 9 aa, Mus musculus
SFYGLSNLR

SEQ ID NO: 45 - protein, 16 aa, Mus musculus
YLEYLNMDDNNIPSTK

SEQ ID NO: 46 - protein, 11 aa, Mus musculus
SNTFTGLVSLK

SEQ ID NO: 47 - protein, 12 aa, Mus musculus
RANPGGPVNFLK

SEQ ID NO: 48 - protein, 11 aa, Mus musculus
SINLGLNNLNK

SEQ ID NO: 49 - protein, 13 aa, Mus musculus
LEPFIFDDQTSLR

SEQ ID NO: 50 - protein, 16 aa, Mus musculus
DVFGPPFQNLNSLDMR

SEQ ID NO: 51 - protein, 18 aa, Mus musculus
DWVWEHFSPMEEQDQSLK

SEQ ID NO: 52 - protein, 17 aa, Mus musculus
DFEAGVLGLEAIVNSIK

SEQ ID NO: 53 - protein, 18 aa, Mus musculus
DFEAGVLGLEAIVNSIKR

SEQ ID NO: 54 - protein, 11 aa, Mus musculus
SHCILNWPVQK

SEQ ID NO: 55 - protein, 8 aa, Mus musculus
LQVALGSR

SEQ ID NO: 56 - protein, 11 aa, Mus musculus
RLNHLEEIDLR

SEQ ID NO: 57 - protein, 10 aa, Mus musculus
LNHLEEIDLR

SEQ ID NO: 58 - protein, 11 aa, Mus musculus
CNCVPVLLGSK

SEQ ID NO: 59 - protein, 15 aa, Mus musculus
LQIRPGSFSGLSDLK

SEQ ID NO: 60 - protein, 11 aa, Mus musculus
PGSFSGLSDLK

SEQ ID NO: 61 - protein, 7 aa, Mus musculus
DAFLVMR

SEQ ID NO: 62 - protein, 24 aa, Mus musculus
KIQENDFNNLNELQVLDLSGNCPR

SEQ ID NO: 63 - protein, 23 aa, Mus musculus
IQENDFNNLNELQVLDLSGNCPR

SEQ ID NO: 64 - protein, 15 aa, Mus musculus
LHSNSLQHVPPTWFK

SEQ ID NO: 65 - protein, 14 aa, Mus musculus
NLQELDLSQNYLAR

SEQ ID NO: 66 - protein, 12 aa, Mus musculus
LEVLDLGTNFIK

SEQ ID NO: 67 - protein, 8 aa, Mus musculus
IADLNIFK

SEQ ID NO: 68 - protein, 6 aa, Mus musculus
HFENLK

SEQ ID NO: 69 - protein, 8 aa, Mus musculus
LIDLSVNK

SEQ ID NO: 70 - protein, 14 aa, Mus musculus
EVGFCPNAQTSVDR

SEQ ID NO: 71 - protein, 13 aa, Mus musculus
HGPQVLEALHYFR

SEQ ID NO: 72 - protein, 7 aa, Mus musculus
NNIFFIK

SEQ ID NO: 73 - protein, 11 aa, Mus musculus
PSDFQHLSFLK

SEQ ID NO: 74 - protein, 8 aa, Mus musculus
YLDFSNNR

SEQ ID NO: 75 - protein, 18 aa, Mus musculus
LLDKLMMNDNDISTSASR

SEQ ID NO: 76 - protein, 14 aa, Mus musculus
LMMNDNDISTSASR

SEQ ID NO: 77 - protein, 8 aa, Mus musculus
TMESDSLR

SEQ ID NO: 78 - protein, 9 aa, Mus musculus
GNHLDVLWR

SEQ ID NO: 79 - protein, 6 aa, Mus musculus
YLDFFK

SEQ ID NO: 80 - protein, 12 aa, Mus musculus
NLFNLEVLDISR

SEQ ID NO: 81 - protein, 19 aa, Mus musculus
NSLNSLPPEVFEGMPPNLK

SEQ ID NO: 82 - protein, 14 aa, Mus musculus
HLEILDLSHNQLTK

SEQ ID NO: 83 - protein, 10 aa, Mus musculus
YFLEDALQLR

SEQ ID NO: 84 - protein, 8 aa, Mus musculus
YLDISSNK

SEQ ID NO: 85 - protein, 6 aa, Mus musculus
IQVIQK

SEQ ID NO: 86 - protein, 15 aa, Mus musculus
NSAVTEWVLQELVAK

SEQ ID NO: 87 - protein, 9 aa, Mus musculus
HFNLCLEER

SEQ ID NO: 88 - protein, 9 aa, Mus musculus
KTVFVMTQK

SEQ ID NO: 89 - protein, 9 aa, Mus musculus
MAFYLSHQR

SEQ ID NO: 90 - protein, 16 aa, Mus musculus
NALTTDNHVAYSQMFK

SEQ ID NO: 91 - protein, 7 aa, Mus musculus
LSLISNR

SEQ ID NO: 92 - protein, 16 aa, Mus musculus
IHHLHNSDFVHLSNLR

SEQ ID NO: 93 - protein, 17 aa, Mus musculus
TLEELNLSYNGITTVPR

SEQ ID NO: 94 - protein, 11 aa, Mus musculus
VLFMDGNCYYK

SEQ ID NO: 95 - protein, 9 aa, Mus musculus
VLDVGGNCR

SEQ ID NO: 96 - protein, 9 aa, Mus musculus
KLNLSFNYR

SEQ ID NO: 97 - protein, 8 aa, Mus musculus
LHLASSFK

SEQ ID NO: 98 - protein, 7 aa, Mus musculus
WLADLPK

SEQ ID NO: 99 - protein, 8 aa, Mus musculus
FVDLSDNR

SEQ ID NO: 100 - protein, 7 aa, Mus musculus
LDLYHWK

SEQ ID NO: 101 - protein, 11 aa, Mus musculus
VSSHLNSNSVR

SEQ ID NO: 102 - protein, 11 aa, Mus musculus
FLDFSGNGMGR

SEQ ID NO: 103 - protein, 21 aa, Mus musculus
LDLSQNNLHILRPQNLDNLPK

SEQ ID NO: 104 - protein, 10 aa, Mus musculus
CGSPGQLQGR

SEQ ID NO: 105 - protein, 8 aa, Mus musculus
SIFAQDLR

SEQ ID NO: 106 - protein, 14 aa, Mus musculus
AQSAVADWVYNELR

SEQ ID NO: 107 - protein, 11 aa, Mus musculus
KTLFVLAHTDR

SEQ ID NO: 108 - protein, 10 aa, Mus musculus
TLFVLAHTDR

SEQ ID NO: 109 - protein, 9 aa, Mus musculus
TSFLLAQQR

SEQ ID NO: 110 - protein, 9 aa, Mus musculus
HFYNQNFCR

SEQ ID NO: 111 - protein, 7 aa, Mus musculus
IDEGAFR

SEQ ID NO: 112 - protein, 14 aa, Mus musculus
TLLLSHNQITHIHK

SEQ ID NO: 113 - protein, 14 aa, Mus musculus
SLVSLTHLSFEGNK

SEQ ID NO: 114 - protein, 8 aa, Mus musculus
VIQNVYLK

SEQ ID NO: 115 - protein, 10 aa, Mus musculus
AMDLSNWELR

SEQ ID NO: 116 - protein, 11 aa, Mus musculus
TVCHLLGNLPK

SEQ ID NO: 117 - protein, 8 aa, Mus musculus
LETLVFQK

SEQ ID NO: 118 - protein, 11 aa, Mus musculus
SFPDFAFSPLK

SEQ ID NO: 119 - protein, 9 aa, Mus musculus
HLEFLSLSR

SEQ ID NO: 120 - protein, 20 aa, Mus musculus
YSFTQFPNLEVLDLGDNNIR

SEQ ID NO: 121 - protein, 11 aa, Mus musculus
TLNHGTFRPLK

SEQ ID NO: 122 - protein, 12 aa, Mus musculus
TLNHGTFRPLKK

SEQ ID NO: 123 - protein, 13 aa, Mus musculus
KLQSLILSHNCLK

SEQ ID NO: 124 - protein, 12 aa, Mus musculus
LQSLILSHNCLK

SEQ ID NO: 125 - protein, 14 aa, Mus musculus
ILEPNSFSGLTNLR

SEQ ID NO: 126 - protein, 9 aa, Mus musculus
TLQYPPFIK

SEQ ID NO: 127 - protein, 11 aa, Mus musculus
LDISGTKDGDR

SEQ ID NO: 128 - protein, 8 aa, Mus musculus
VINQSHLK

SEQ ID NO: 129 - protein, 11 aa, Mus musculus
SLMFFDVYGNK

SEQ ID NO: 130 - protein, 11 aa, Mus musculus
LQCTCDNLWFK

SEQ ID NO: 131 - protein, 6 aa, Mus musculus
AWYLTK

SEQ ID NO: 132 - protein, 15 aa, Mus musculus
ELVPALEQGSQTTFK

SEQ ID NO: 133 - protein, 6 aa, Mus musculus
LCLHQR

SEQ ID NO: 134 - protein, 9 aa, Mus musculus
LEVQLASMK

SEQ ID NO: 135 - protein, 6 aa, Mus musculus
MFYEHK

SEQ ID NO: 136 - protein, 14 aa, Mus musculus
ETVEKENTHLIVVE